(19) United States
(12) Patent Application Publication
(10) Pub. No.: US 2005/0250133 A1
(43) Pub. Date: Nov. 10, 2005

(54) Molecular interaction sites of 16S ribosomal RNA and methods of modulating the same

(76) Inventors: David J. Ecker, Encinitas, CA (US); Steven A. Hofstadler, Vista, CA (US); Richard H. Griffey, Vista, CA (US); Rangarajan Sampath, San Diego, CA (US); Kristin Sannes Lowery, Vista, CA (US); Christian Massire, Carlsbad, CA (US)

Correspondence Address: COZEN O'CONNOR, P.C., 1900 MARKET STREET, PHILADELPHIA, PA, 19103-3508, US

(21) Appl. No.: 11/070,519

(22) Filed: Mar. 1, 2005

Related U.S. Application Data
(63) Continuation-in-part of application No. 10/223,126, filed on Aug. 16, 2002.
(60) Provisional application No. 60/313,890, filed on Aug. 21, 2001.

(51) Int. Cl. C12Q 1/68; G06F 19/00; G01N 33/48; G01N 33/50

(52) U.S. Cl. 435/6; 702/20

(57) ABSTRACT

Polynucleotides comprising molecular interaction sites of 16S rRNA that have particular secondary structure are provided. Methods of using such polynucleotides to screen, virtually or actually, combinatorial libraries of compounds that bind thereto are also provided. Method of modulating the activity of 16S rRNA by contacting 16S rRNA or prokaryotic cells containing the same with a compound identified by such virtual or actual screening are also provided.

### CROSS-REFERENCE TO RELATED APPLICATIONS

This application claims priority to U.S. application Ser. No. 10/223,126 filed Aug. 16, 2002, which claims priority to U.S. provisional application Ser. No. 60/313,890 filed Aug. 21, 2001. This application also claims priority to U.S. application Ser. No. 10/223,156 filed Aug. 16, 2002, which claims priority to U.S. provisional application Ser. No. 60/314,020 filed Aug. 21, 2001. This application also claims priority to U.S. application Ser. No. 10/225,501 filed Aug. 19, 2002, which claims priority to U.S. provisional application Ser. No. 60/314,236 filed Aug. 22, 2001. This application also claims priority to U.S. application Ser. No. 10/224,836 filed Aug. 20, 2002, which claims priority to U.S. provisional application Ser. No. 60/314,251 filed Aug. 22, 2001. This application also claims priority to U.S. application Ser. No. 10/864,999 filed Jun. 10, 2004, which claims priority to U.S. provisional application Ser. No. 60/477,821 filed Jun. 12, 2003. The entire contents of these applications is incorporated herein by reference in their entirety.

### FIELD OF THE INVENTION

The present invention relates to identification of molecular interaction sites of 16S rRNA, virtual or actual screening of compounds that bind thereto, and to modulating the activity of 16S rRNA with such compounds identified in the actual or virtual screening.

### BACKGROUND OF THE INVENTION

Ribosomes are large, multisubunit ribonucleoprotein complexes (RNPs) that are responsible for protein synthesis, and are highly conserved, both structurally and functionally, across microbial phyla. They include large (50S) and small (30S) subunits that are assembled from ribosomal RNAs (rRNAs) and proteins bound to the rRNA. The 30S ribosomal subunit contains the 16S rRNA. Ribosomes synthesize proteins when correctly bound to messenger RNA (mRNA) and transfer RNA (tRNA). It is now generally accepted that the sites of action of numerous antimicrobial compounds that inhibit ribosomes lie within 16S rRNA. Very large molecules such as ribosomes are not, however, usually desirable targets for high-throughput screens. Several factors related to the structural complexity of the ribosome complicate screening assays that rely on binding of a potential drug candidate to a ribosomal target, including difficulty in obtaining large quantities of purified ribosomes and degradation of ribosomes under typical screening conditions.

Proper assembly of the various components involved in protein synthesis is thought to be directed by binding sites on the rRNAs. The A and P sites, which are binding sites on the rRNA important for protein synthesis, accommodate the incoming aminoacyl-tRNA (A site) and the peptidyl-tRNA (P site), respectively. In prokaryotes, these sites are composed of, in part, highly ordered structures of the 16S rRNA, most likely in the cleft of the 30S subunit. Although the primary nucleotide sequences of rRNA molecules differ, ribosomes are structurally similar in all species (including eukaryotes). Noller, In The RNA World, Gesteland and Atkins (eds.), 137-84 (CSHL Press, New York, 1993); Noller et al., In The Ribosome: Structure, Function, and Evolution, Hill, et al. (eds.), 73-92 (Amer. Soc. for Microbiol., Washington, D.C., 1990).

It is now generally accepted that 16S and 23S rRNAs (found in the 50S ribosomal subunit; analogous eukaryotic rRNAs are 28S, 5.8S, and 5S rRNA in the 60S ribosomal subunit, and 18S rRNA in the 40S ribosomal subunit) play important, if not critical, roles in the decoding and peptidyl transferase activities of ribosomes. Most antibiotics that inhibit protein synthesis act directly on ribosomes. Aminoglycoside antibiotics interact with sites on ribosomal subunits resulting in protection of RNA as visualized by RNA footprint assays. Moazed et al., Nature, 1987, 327, 389; Woodcock et al., EMBO J. 1991, 10, 3099; and Thompson et al., Biochimie, 1991, 73, 1131. Specific nucleotides in 16S rRNA are the binding targets of aminoglycosides such as neomycin, streptomycin, hygromycin, gentamycin, and tetracycline. Similarly, specific nucleotides in 23S rRNA are targeted by numerous MLS compounds (macrolides, lincomycins, and streptogramins), including erythromycin.

Some antibiotics (e.g., edeine, pactamycin, apramycin, and neamine) inhibit protein synthesis by interfering with binding between tRNA and the A- or P-sites on the ribosome during translation. Woodcock et al., EMBO J. 1991, 10, 3099. Interactions of many of these compounds with 16S rRNA in the 30S ribosomal subunit have been mapped to various functional sites, primarily by chemical footprinting assays. Other antibiotic compounds, such as the peptide antibiotic, thiostrepton, have been shown to similarly interact with 23S rRNA in 50S subunits (Thompson et al., 1991, supra).

The oligonucleotide analog approach provides a useful alternative strategy in such applications by effectively subdividing large RNP's into small protein-free subdomains that, to some significant extent, recapitulate the functional properties of the analogous regions of the intact RNP. Implicit in this approach are the notions that the RNP (in this case the ribosome) is essentially an RNA machine, and that most, if not all, of the associated (ribosomal) proteins perform essentially a chaperonin function, by helping to guide the folding of the large and complexly structured rRNA. The feasibility of the oligonucleotide analog strategy has already been demonstrated with analogs of the decoding region of 16S rRNA, which recapitulate aminoglycoside antibiotic binding (and other) interactions of the small (30S) subunit of the ribosome.

Much of the 5′-domain, most of the central domain, and parts of the 3′-domain of 16S rRNA are not involved directly in functional interactions. In addition to the decoding region, which contains adjacent A- and P-site subdomains, functional sites are distributed throughout this “functional skeleton.” Interestingly, while A-site associated nucleotides are concentrated in only two regions, the decoding region and the 530 stem-loop, P-site associated nucleotides are widely distributed. Presumably, folding of 16S RNA brings these sites together in three-dimensions to form the single P-site of the 30S subunit. In this connection, nucleotides in the 690 stem-loop, the 790 stem-loop, and G926 Helix appear to form partial binding sites for the oligopeptide antibiotic edeine, as this P-site-specific antibiotic protects bases in all of these locations. Twenty 30S subunit proteins (S2-S21) assemble with 16S rRNA. Three types of nucleotides can be categorized: i) nucleotides that remain reactive towards single-strand-specific chemical probes (DMS, kethoxal, CMCT) in assembled 30S subunits, ii) nucleotides with backbone protected from hydroxy radical attack by specific proteins, and iii) nucleotides protected from attack by single-strand-specific chemical probes by specific proteins. Functionally implicated rRNA sequences tend to be highly conserved and usually offer little potential, in themselves, for bacteria-specific drug targeting. Significant sequence diversity may exist at positions immediately surrounding highly conserved sites, however, and these more diverse positions may serve to enable organism-specific targeting of compounds (drugs) to closely associated functional sites. The present invention identifies subdomains of 16S rRNA that can act as targets for ribosome-targeted antimicrobial drug discovery.

Recent advances in genomics, molecular biology, and structural biology have highlighted how RNA molecules participate in or controls many of the events required to express proteins in cells. Rather than function as simple intermediaries, RNA molecules actively regulate their own transcription from DNA, splice and edit MRNA molecules and tRNA molecules, synthesize peptide bonds in the ribosome, catalyze the migration of nascent proteins to the cell membrane, and provide fine control over the rate of translation of messages. RNA molecules can adopt a variety of unique structural motifs that provide the framework required to perform these functions.

“Small” molecule therapeutics, which bind specifically to structured RNA molecules, are organic chemical molecules that are not polymers. “Small” molecule therapeutics include, for example, the most powerful naturally-occurring antibiotics. For example, the aminoglycoside and macrolide antibiotics are “small” molecules that bind to defined regions in ribosomal RNA (rRNA) structures and work, it is believed, by blocking conformational changes in the RNA required for protein synthesis. In addition, changes in the conformation of RNA molecules have been shown to regulate rates of transcription and translation of mRNA molecules. Small molecules are generally less than 10 kDa.

RNA molecules or groups of related RNA molecules are believed by Applicants to have regulatory regions that are used by the cell to control synthesis of proteins. The cell is believed to exercise control over both the timing and the amount of protein that is synthesized by direct, specific interactions with RNA. This notion is inconsistent with the impression obtained by reading the scientific literature on gene regulation, which is highly focused on transcription. The process of RNA maturation, transport, intracellular localization and translation are rich in RNA recognition sites that provide good opportunities for drug binding. Applicants' invention is directed, inter alia, to finding these regions of RNA molecules, in particular the 16S rRNA, in the microbial genome. Applicants' invention also makes use of combinatorial chemistry to make and/or screen, actually or virtually, a large number of chemical entities for their ability to bind and/or modulate these drug binding sites.

The determination of potential three dimensional structures of nucleic acids and their attendant structural motifs affords insights into areas such as the study of catalysis by RNA, RNA-RNA interactions, RNA-nucleic acid interactions, RNA-protein interactions, and the recognition of small molecules by nucleic acids. Four general approaches to the generation of model three dimensional structures of RNA have been demonstrated in the literature. All of these employ sophisticated molecular modelling and computational algorithms for the simulation of folding and tertiary interactions within target nucleic acids, such as RNA. Westhof and Altman (Proc. Natl. Acad. Sci., 1994, 91, 5133, incorporated herein by reference in its entirety) have described the generation of a three-dimensional working model of M1 RNA, the catalytic RNA subunit of RNase P from E. coli via an interactive computer modelling protocol. Leveraging the significant body of work in the area of cryo-electron microscopy (cryo-EM) and biochemical studies on ribosomal RNAs, Mueller and Brimacombe (J. Mol. Biol, 1997, 271, 524) have constructed a three dimensional model of E. coli 16S Ribosomal RNA. A method to model nucleic acid hairpin motifs has been developed based on a set of reduced coordinates for describing nucleic acid structures and a sampling algorithm that equilibrates structures using Monte Carlo (MC) simulations (Tung, Biophysical J. 1997, 72, 876, incorporated herein by reference in its entirety). MC-SYM is yet another approach to predicting the three dimensional structure of RNAs using a constraint-satisfaction method. Major et al., Proc. Nati. Acad. Sci., 1993, 90, 9408. The MC-SYM program is an algorithm based on constraint satisfaction that searches conformational space for all models that satisfy query input constraints, and is described in, for example, Cedergren et al., RNA Structure And Function, 1998, Cold Spring Harbor Lab. Press, p.37-75. Three dimensional structures of RNA are produced by that method by the stepwise addition of nucleotide having one or several different conformations to a growing oligonucleotide model.

Westhof and Altman (Proc. Natl. Acad. Sci., 1994, 91, 5133) have described the generation of a three-dimensional working model of M1 RNA, the catalytic RNA subunit of RNase P from E. coli via an interactive computer modelling protocol. This modelling protocol incorporated data from chemical and enzymatic protection experiments, phylogenetic analysis, studies of the activities of mutants and the kinetics of reactions catalyzed by the binding of substrate to M1 RNA. Modelling was performed for the most part as described in the literature. Westhof et al., in “Theoretical Biochemistry and Molecular Biophysics,” Beveridge and Lavery (Eds.), Adenine, N.Y., 1990, 399. In general, starting with the primary sequence of M1 RNA, the stem-loop structures and other elements of secondary structure were created. Subsequent assembly of these elements into a three dimensional structure using a computer graphics station and FRODO (Jones, J. Appl. Crystallogr., 1978, 11, 268) followed by refinement using NUCLIN-NUCLSQ afforded a RNA model that had correct geometries, the absence of bad contacts, and appropriate stereochemistry. The model so generated was found to be consistent with a large body of empirical data on M1 RNA and opens the door for hypotheses about the mechanism of action of RNase P. The models generated by this method, however, are less well resolved that the structures determined via X-ray crystallography.

Mueller and Brimacombe (J. Mol. Biol., 1997, 271, 524, which is incorporated herein by reference in its entirety) have constructed a three dimensional model of E. coli 16S ribosomal RNA using a modelling program called ERNA-3D. This program generates three dimensional structures such as A-form RNA helices and single-strand regions via the dynamic docking of single strands to fit electron density obtained from low resolution diffraction data. After helical elements have been defined and positioned in the model, the configurations of the single strand regions is adjusted, so as to satisfy any known biochemical constraints such as RNA-protein cross-linking and foot-printing data.

A method to model nucleic acid hairpin motifs has been developed based on a set of reduced coordinates for describing nucleic acid structures and a sampling algorithm that equilibrates structures using Monte Carlo (MC) simulations. Tung, Biophysical J. 1997, 72, 876, incorporated herein by reference in its entirety. The stem region of a nucleic acid can be adequately modelled by using a canonical duplex formation. Using a set of reduced coordinates, an algorithm that is capable of generating structures of single stranded loops with a pair of fixed ends was created. This allows efficient structural sampling of the loop in conformational space. Combining this algorithm with a modified Metropolis Monte Carlo algorithm afforded a structure simulation package that simplifies the study of nucleic acid hairpin structures by computational means. Once the RNA subdomains have been identified, they can, if desired, be stabilized by the methods disclosed in U.S. Pat. No. 5,712,096.

While X-ray crystallography is a very powerful technique that can allow for the determination of some secondary and tertiary structure of biopolymeric targets (Erikson et al., Ann. Rep. in Med. Chem., 1992, 27, 271-289), this technique can be an expensive procedure and very difficult to accomplish. Crystallization of biopolymers is extremely challenging, difficult to perform at adequate resolution, and is often considered to be as much an art as a science. Further confounding the utility of X-ray crystal structures in the drug discovery process is the inability of crystallography to reveal insights into the solution-phase, and therefore the biologically relevant, structures of the targets of interest. Some analysis of the nature and strength of interaction between a ligand (agonist, antagonist, or inhibitor) and its target can be performed by ELISA (Kemeny and Challacombe, in ELISA and other Solid Phase Immunoassays: 1988), radioligand binding assays (Berson et al., Clin. 1968; Chard, in “An Introduction to Radioimmunoassay and Related Techniques,” 1982), surface-plasmon resonance (Karlsson et al., 1991, Jonsson et al., Biotechniques, 1991), or scintillation proximity assays (Udenfriend et al., Anal. Biochem., 1987), all cited previously. The radioligand binding assays are typically useful only when assessing the competitive binding of the unknown at the binding site for that of the radioligand and also require the use of radioactivity. The surface-plasmon resonance technique is more straightforward to use, but is also quite costly. Conventional biochemical assays of binding kinetics, and dissociation and association constants are also helpful in elucidating the nature of the target-ligand interactions.

Accordingly, one aspect of the invention identifies molecular interaction sites in 16S rRNA. These molecular interaction sites, which comprise secondary structural elements, are highly likely to give rise to significant therapeutic, regulatory, or other interactions with “small” molecules and the like. Another aspect of the invention is to compare molecular interaction sites of 16S rRNA with compounds proposed for interaction therewith.

Yet another aspect of the present invention is the establishment of databases of the numerical representations of three-dimensional structures of molecular interaction sites of 16S rRNA. Such databases libraries provide powerful tools for the elucidation of structure and interactions of molecular interaction sites with potential ligands and predictions thereof. Another aspect of the present invention is to provide a general method for the screening of combinatorial libraries comprising individual compounds or mixtures of compounds against 16S rRNA, so as to determine which components of the library bind to the target.

### SUMMARY OF THE INVENTION

The present invention is directed to identification of molecular interaction sites of 16S rRNA that comprise particular secondary structure.

The present invention is also directed to nucleic acid molecules, polynucleotides or oligonucleotides comprising the molecular interaction sites that can be used to screen, virtually or actually, combinatorial libraries of compounds that bind thereto.

The present invention is also directed to computer-readable medium comprising three dimensional representations of the structures of the molecular interaction sites.

The present invention is also directed to methods of identifying compounds which bind to molecular interaction sites of 16S rRNA through either virtual or actual screening of a plurality of compounds.

The present invention is also directed to modulating the activity of 16S rRNA by contacting 16S rRNA or prokaryotic cells comprising the same with a compound identified by such virtual or actual screening.

The present invention is also directed to modulating prokaryotic cell growth comprising contacting a prokaryotic cell with a compound identified by such virtual or actual screening.

### BRIEF DESCRIPTION OF THE DRAWINGS

FIGS. 1, 1A, 1B, and 1C depict representative secondary structures of consensus 16S rRNA showing consensus sites 1-19 (nucleotides: capitalized letters=>95% conservation; small letters=90 to 95% conservation; ●=80 to 90% conservation; and ∘=<80% conservation; bonds: —=Watson-Crick bond; ● and ∘=non-cannonical bonds).

FIGS. 2, 2A, 2B and 2C depict representative secondary structures of consensus 16S rRNA showing consensus sites 20-28 (nucleotides: capitalized letters=>95% conservation; small letters=90 to 95% conservation; ●=80 to 90% conservation; and ∘=<80% conservation; bonds: —=Watson-Crick bond; ● and ∘=non-cannonical bonds).

FIGS. 3, 3A, 3B and 3C depict representative secondary structures of consensus 16S rRNA showing consensus sites 29-39 (nucleotides: capitalized letters=>95% conservation; small-letters=90 to 95% conservation; ●=80 to 90% conservation; and ∘=<80% conservation; bonds: —=Watson-Crick bond; ● and ∘=non-cannonical bonds).

FIG. 4 depicts a representative secondary structure of consensus 16S rRNA showing consensus sites 40-42 (nucleotides: capitalized letters=>95% conservation; small letters=90 to 95% conservation; ●=80 to 90% conservation; and ∘=<80% conservation; bonds: —=Watson-Crick bond; ● and ∘=non-cannonical bonds).

### DESCRIPTION OF PREFERRED EMBODIMENTS OF THE INVENTION

The present invention is directed to, inter alia, identification of molecular interaction sites of 16S rRNA. Such molecular interaction sites comprise secondary structure capable of interacting with cellular components, such as factors and proteins required for translation and other cellular processes. Nucleic acid molecules or polynucleotides comprising the molecular interaction sites can be used to screen, virtually or actually, combinatorial libraries of compounds that bind thereto. The compounds identified by such screening are used to modulate the activity of 16S rRNA and, thus, can be used to modulate, either inhibit or stimulate, prokaryotic cell growth. Thus, novel drugs, agricultural chemicals, industrial chemicals and the like that operate through the modulation of 16S rRNA can be identified.

A number of procedures and protocols are preferably integrated to provide powerful drug and other biologically useful compound identification. Pharmaceuticals, veterinary drugs, agricultural chemicals, pesticides, herbicides, fungicides, industrial chemicals, research chemicals and many other beneficial compounds useful in pollution control, industrial biochemistry, and biocatalytic systems can be identified in accordance with embodiments of this invention. Novel combinations of procedures provide extraordinary power and versatility to the present methods. While it is preferred in some embodiments to integrate a number of processes developed by the assignee of the present application as will be set forth more fully herein, it should be recognized that other methodologies can be integrated herewith to good effect. Thus, while it is greatly advantageous to determine molecular binding sited on 16S rRNA in accordance with the teachings of this invention, the interactions of ligands and libraries of ligands with other 16S rRNA identified as being of interest may greatly benefit from other aspects of this invention. All such combinations are within the spirit of the invention.

One aspect of Applicants' invention is directed to identifying secondary structures in 16S rRNA termed “molecular interaction sites.” As used herein, “molecular interaction sites” are regions of 16S rRNA that have secondary structure. Molecular interaction sites can be conserved among a plurality of different taxonomic species of 16S rRNA. Molecular interaction sites are small, preferably less than 200 nucleotides, preferably less than 150 nucleotides, preferably less than 70 nucleotides, preferably less than 50 nucleotides, alternatively less than 30 nucleotides, independently folded, functional subdomains contained within a larger RNA molecule. Molecular interaction sites can contain both single-stranded and double-stranded regions. Thus, molecular interaction sites are capable of undergoing interaction with “small” molecules and otherwise, and are expected to serve as sites for interacting with “small” molecules, oligomers such as oligonucleotides, and other compounds in therapeutic and other applications. Molecular interaction sites also comprise a pocket for binding small molecules, drugs and the like.

The molecular interaction sites are present within at least 16S rRNA. In accordance with some embodiments of this invention, it will be appreciated that the 16S rRNAs having a molecular interaction site or sites may be derived from a number of sources. Thus, such 16S rRNAs can be identified by any means, rendered into three dimensional representations and employed for the identification of compounds that can interact with them to effect modulation of the 16S rRNA. In some embodiments, the molecular interaction sites that are identified in 16S rRNA are absent from eukaryotes, particularly humans, and, thus, can serve as sites for “small” molecule binding with concomitant modulation of the 16S rRNA of prokaryotic organisms without effecting human toxicity.

The molecular interaction sites can be identified by any means known to the skilled artisan. In some embodiments of the invention, the molecular interaction sites in 16S rRNA are identified according to the general methods described in International Publication WO 99/58719, which is incorporated herein by reference in its entirety. Briefly, a target 16S rRNA nucleotide sequence is chosen from among known sequences. Any 16S rRNA nucleotide sequence can be chosen. The nucleotide sequence of the target 16S rRNA is compared to the nucleotide sequences of a plurality of 16S rRNAs from different taxonomic species. At least one sequence region that is effectively conserved among the plurality of 16S rRNAs and the target 16S rRNA is identified. Such conserved region is examined to determine whether there is any secondary structure, and, for conserved regions having secondary structure, such secondary structure is identified.

In accordance with some embodiments of the invention, the nucleotide sequence of the target 16S rRNA is compared with the nucleotide sequences of a plurality of corresponding 16S rRNAs from different taxonomic species. Initial selection of a particular target nucleic acid can be based upon any functional criteria. 16S rRNA known to be involved in pathogenic genomes such as, for example, bacterial and yeast, are exemplary targets. Pathogenic bacteria and yeast are well known to those skilled in the art. Additional 16S rRNA targets can be determined independently or can be selected from publicly available prokaryotic genetic databases known to those skilled in the art. Databases include, for example, Online Mendelian Inheritance in Man (OMIM), the Cancer Genome Anatomy Project (CGAP), GenBank, EMBL, PIR, SWISS-PROT, and the like. OMIM, which is a database of genetic mutations associated with disease, was developed, in part, for the National Center for Biotechnology Information (NCBI). OMIM can be accessed through the world wide web of the Internet at, for example, ncbi.nlm.nih.gov/Omim/. CGAP, which is an interdisciplinary program to establish the information and technological tools required to decipher the molecular anatomy of a cancer cell, can be accessed through the world wide web of the Internet at, for example, ncbi.nlm.nih.gov/ncicgap/. Some of these databases may contain complete or partial nucleotide sequences. In addition, 16S rRNA targets can also be selected from private genetic databases. Alternatively, 16S rRNA targets can be selected from available publications or can be determined especially for use in connection with the present invention.

After a 16S rRNA target is selected or provided, the nucleotide sequence of the 16S rRNA target is determined and then compared to the nucleotide sequences of a plurality of 16S rRNAs from different taxonomic species. In one embodiment of the invention, the nucleotide sequence of the 16S rRNA target is determined by scanning at least one genetic database or is identified in available publications. Databases known and available to those skilled in the art include, for example, GenBank, and the like. These databases can be used in connection with searching programs such as, for example, Entrez, which is known and available to those skilled in the art, and the like. Entrez can be accessed through the world wide web of the Internet at, for example, ncbi.nlm.nih.gov/Entrez/. Preferably, the most complete nucleic acid sequence representation available from various databases is used. The GenBank database, which is known and available to those skilled in the art, can also be used to obtain the most complete nucleotide sequence. GenBank is the NIH genetic sequence database and is an annotated collection of all publicly available DNA sequences. GenBank is described in, for example, Nuc. Acids Res., 1998, 26, 1-7, which is incorporated herein by reference in its entirety, and can be accessed by those skilled in the art through the world wide web of the Internet at, for example, ncbi.nlm.nih.gov/Web/Genbank/index.html. Alternatively, partial nucleotide sequences of 16S rRNA targets can be used when a complete nucleotide sequence is not available.

The nucleotide sequence of the 16S rRNA target is compared to the nucleotide sequences of a plurality of 16S rRNAs from different taxonomic species. A plurality of 16S rRNAs from different taxonomic species, and the nucleotide sequences thereof, can be found in genetic databases, from available publications, or can be determined especially for use in connection with the present invention. In one embodiment of the invention, the 16S rRNA target is compared to the nucleotide sequences of a plurality of 16S rRNAs from different taxonomic species by performing a sequence similarity search, an ortholog search, or both, such searches being known to persons of ordinary skill in the art.

The result of a sequence similarity search is a plurality of 16S rRNAs having at least a portion of their nucleotide sequences which are homologous to at least an 8 to 20 nucleotide region of the target 16S rRNA, referred to as the window region. Preferably, the plurality of 16S rRNAs comprise at least one portion which is at least 60% homologous to any window region of the target 16S rRNA. More preferably, the homology is at least 70%. More preferably, the homology is at least 80%. Most preferably, the homology is at least 90% or 95%. For example, the window size, the portion of the target 16S rRNA to which the plurality of sequences are compared, can be from about 8 to about 20, preferably from about 10 to about 15, most preferably from about 11 to about 12, contiguous nucleotides. The window size can be adjusted accordingly. A plurality of 16S rRNAs from different taxonomic species is then preferably compared to each likely window in the target 16S rRNA until all portions of the plurality of sequences is compared to the windows of the target 16S rRNA. Sequences of the plurality of 16S rRNAs from different taxonomic species which have portions which are at least 60%, preferably at least 70%, more preferably at least 80%, or most preferably at least 90% homologous to any window sequence of the target 16S rRNA are considered as likely homologous sequences.

Sequence similarity searches can be performed manually or by using several available computer programs known to those skilled in the art. Preferably, Blast and Smith-Waterman algorithms, which are available and known to those skilled in the art, and the like can be used. Blast is NCBI's sequence similarity search tool designed to support analysis of nucleotide and protein sequence databases. Blast can be accessed through the world wide web of the Internet at, for example, ncbi.nlm.nih.gov/BLAST/. The GCG Package provides a local version of Blast that can be used either with public domain databases or with any locally available searchable database. GCG Package v.9.0 is a commercially available software package that contains over 100 interrelated software programs that enables analysis of sequences by editing, mapping, comparing and aligning them. Other programs included in the GCG Package include, for example, programs which facilitate RNA secondary structure predictions, nucleic acid fragment assembly, and evolutionary analysis. In addition, the most prominent genetic databases (GenBank, EMBL, PIR, and SWISS-PROT) are distributed along with the GCG Package and are fully accessible with the database searching and manipulation programs. GCG can be accessed through the world wide web of the Internet at, for example, gcg.com/. Fetch is a tool available in GCG that can get annotated GenBank records based on accession numbers and is similar to Entrez. Another sequence similarity search can be performed with GeneWorld and GeneThesaurus from Pangea. GeneWorld 2.5 is an automated, flexible, high-throughput application for analysis of polynucleotide and protein sequences. GeneWorld allows for automatic analysis and annotations of sequences. Like GCG, GeneWorld incorporates several tools for homology searching, gene finding, multiple sequence alignment, secondary structure prediction, and motif identification. GeneThesaurus 1.0™ is a sequence and annotation data subscription service providing information from multiple sources, providing a relational data model for public and local data.

Another alternative sequence similarity search can be performed, for example, by BlastParse. BlastParse is a PERL script running on a UNIX platform that automates the strategy described above. BlastParse takes a list of target accession numbers of interest and parses all the GenBank fields into “tab-delimited” text that can then be saved in a “relational database” format for easier search and analysis, which provides flexibility. The end result is a series of completely parsed GenBank records that can be easily sorted, filtered, and queried against, as well as an annotations-relational database.

Another toolkit capable of doing sequence similarity searching and data manipulation is SEALS, also from NCBI. This tool set is written in perl and C and can run on any computer platform that supports these languages. It is available for download, for example, at the world wide web of the Internet at ncbi.nlm.nih.gov/Walker/SEALS/. This toolkit provides access to Blast2 or gapped blast. It also includes a tool called tax_collector which, in conjunction with a tool called tax break, parses the output of Blast2 and returns the identifier of the sequence most homologous to the query sequence for each species present. Another useful tool is feature2fasta which extracts sequence fragments from an input sequence based on the annotation.

Preferably, the plurality of 16S rRNAs from different taxonomic species which have homology to the target nucleic acid, as described above in the sequence similarity search, are further delineated so as to find orthologs of the target 16S rRNA therein. An ortholog is a term defined in gene classification to refer to two genes in widely divergent organisms that have sequence similarity, and perform similar functions within the context of the organism. In contrast, paralogs are genes within a species that occur due to gene duplication, but have evolved new functions, and are also referred to as isotypes. Optionally, paralog searches can also be performed. By performing ant ortholog search, an exhaustive list of homologous sequences from diverse organisms is obtained. Subsequently, these sequences are analyzed to select the best representative sequence that fits the criteria for being an ortholog. An ortholog search can be performed by programs available to those skilled in the art including, for example, Compare. Preferably, an ortholog search is performed with access to complete and parsed GenBank annotations for each of the sequences. Currently, the records obtained from GenBank are “flat-files”, and are not ideally suited for automated analysis. Preferably, the ortholog search is performed using a Q-Compare program. The Blast Results-Relation database and the Annotations-Relational database are used in the Q-Compare protocol, which results in a list of ortholog sequences to compare in the interspecies sequence comparisons programs described below.

The above-described similarity searches provide results based on cut-off values, referred to as e-scores. E-scores represent the probability of a random sequence match within a given window of nucleotides. The lower the e-score, the better the match. One skilled in the art is familiar with e-scores. The user defines the e-value cut-off depending upon the stringency, or degree of homology desired, as described above. In some embodiments of the invention, it is preferred that any homologous nucleotide sequences of 16S rRNA that are identified not be present in the human genome.

In another embodiment of the invention, the sequences required are obtained by searching ortholog databases. One such database is Hovergen, which is a curated database of vertebrate orthologs. Ortholog sets may be exported from this database and used as is, or used as seeds for further sequence similarity searches as described above. Further searches may be desired, for example, to find invertebrate orthologs. Hovergen can be downloaded as a file transfer program at, for example, pbil.univlyon l.fr/pub/hovergen/. A database of prokaryotic orthologs, COGS, is available and can be used interactively through the world wide web of the Internet at, for example, ncbi.nlm.nih.gov/COG/.

After the orthologs or virtual transcripts described above are obtained through either the sequence similarity search or the ortholog search, at least one sequence region which is conserved among the plurality of 16S rRNAs from different taxonomic species and the target 16S rRNA is identified. Interspecies sequence comparisons can be performed using numerous computer programs which are available and known to those skilled in the art. Preferably, interspecies sequence comparison is performed using Compare, which is available and known to those skilled in the art. Compare is a GCG tool that allows pair-wise comparisons of sequences using a window/stringency criterion. Compare produces an output file containing points where matches of specified quality are found. These can be plotted with another GCG tool, DotPlot.

Alternatively, the identification of a conserved sequence region is performed by interspecies sequence comparisons using the ortholog sequences generated from Q-Compare in combination with CompareOverWins. Preferably, the list of sequences to compare, i.e., the ortholog sequences, generated from Q-Compare is entered into the CompareOverWins algorithm. Preferably, interspecies sequence comparisons are performed by a pair-wise sequence comparison in which a query sequence is slid over a window on the master target sequence. Preferably, the window is from about 9 to about 99 contiguous nucleotides.

Sequence homology between the window sequence of the target 16S rRNA and the query sequence of any of the plurality of 16S rRNAs obtained as described above, is preferably at least 60%, more preferably at least 70%, more preferably at least 80%, and most preferably at least 90% or 95%. The most preferable method of choosing the threshold is to have the computer automatically try all thresholds from 50% to 100% and choose a threshold based a metric provided by the user. One such metric is to pick the threshold such that exactly n hits are returned, where n is usually set to 3. This process is repeated until every base on the query nucleic acid, which is a member of the plurality of 16S rRNAs described above, has been compared to every base on the master target sequence. The resulting scoring matrix can be plotted as a scatter plot. Based on the match density at a given location, there may be no dots, isolated dots, or a set of dots so close together that they appear as a line. The presence of lines, however small, indicates primary sequence homology. Sequence conservation within 16S rRNA in divergent species is likely to be an indicator of conserved regulatory elements that are also likely to have a secondary structure. The results of the interspecies sequence comparison can be analyzed using MS Excel and visual basic tools in an entirely automated manner as known to those skilled in the art.

After at least one region that is conserved between the nucleotide sequence of the 16S rRNA target and the plurality of 16S rRNAs from different taxonomic species, preferably via the orthologs, is identified, the conserved region is analyzed to determine whether it contains secondary structure. Determining whether the identified conserved regions contain secondary structure can be performed by a number of procedures known to those skilled in the art. Determination of secondary structure is preferably performed by self complementarity comparison, alignment and covariance analysis, secondary structure prediction, or a combination thereof.

In one embodiment of the invention, secondary structure analysis is performed by alignment and covariance analysis. Numerous protocols for alignment and covariance analysis are known to those skilled in the art. Preferably, alignment is performed by ClustalW, which is available and known to those skilled in the art. ClustalW is a tool for multiple sequence alignment that, although not a part of GCG, can be added as an extension of the existing GCG tool set and used with local sequences. ClustalW can be accessed through the world wide web of the Internet at, for example, dot.imgen.bcm.tmc.edu:9331/multi-align/Options/clustalw.html. ClustalW is also described in Thompson, et al., Nuc. Acids Res., 1994, 22, 4673-4680, which is incorporated herein by reference in its entirety. These processes can be scripted to automatically use conserved UTR regions identified in earlier steps. Seqed, a UNIX command line interface available and known to those skilled in the art, allows extraction of selected local regions from a larger sequence. Multiple sequences from many different species can be clustered and aligned for further analysis.

In another embodiment of the invention, the output of all possible pair-wise CompareOverWindows comparisons are compiled and aligned to a reference sequence using a program called AlignHits, a program that can be reproduced by one skilled in the art. One purpose of this program is to map all hits made in pair-wise comparisons back to the position on a reference sequence. This method combining CompareOverWindows and AlignHits provides more local alignments (over 20-100 bases) than any other algorithm. This local alignment is required for the structure finding routines described later such as covariation or RevComp. This algorithm writes a fasta file of aligned sequences. It is important to differentiate this from using ClustalW by itself, without CompareOverWindows and AlignHits.

Covariation is a process of using phylogenetic analysis of primary sequence information for consensus secondary structure prediction. Covariation is described in the following references, each of which is incorporated herein by reference in their entirety: Gutell et al., “Comparative Sequence Analysis Of Experiments Performed During Evolution” In Ribosomal RNA Group I Introns, Green, Ed., Austin: Landes, 1996; Gautheret et al., Nuc. Acids Res., 1997, 25, 1559-1564; Gautheret et al., RNA, 1995, 1, 807-814; Lodmell et al., Proc. Natl. Acad. Sci. USA, 1995, 92, 10555-10559; Gautheret et al., J. Mol. Biol., 1995, 248, 27-43; Gutell, Nuc. Acids Res., 1994, 22, 3502-3517; Gutell, Nuc. Acids Res., 1993, 21, 3055-3074; Gutell, Nuc. Acids Res., 1993, 21, 3051-3054; Woese, Proc. Natl. Acad. Sci. USA, 1989, 86, 3119-3122; and Woese et al., Nuc. Acids Res., 1980, 8, 2275-2293, each of which is incorporated herein by reference in its entirety. Preferably, covariance software is used for covariance analysis. Preferably, Covariation, a set of programs for the comparative analysis of RNA structure from sequence alignments, is used. Covariation uses phylogenetic analysis of primary sequence information for consensus secondary structure prediction. Covariation can be obtained through the world wide web of the Internet at, for example, mbio.ncsu.edu/RNaseP/info/programs/programs.html. A complete description of a version of the program has been published (Brown, J. W. 1991, Phylogenetic analysis of RNA structure on the Macintosh computer. CABIOS 7:391-393). The current version is v4.1, which can perform various types of covariation analysis from RNA sequence alignments, including standard covariation analysis, the identification of compensatory base-changes, and mutual information analysis. The program is well-documented and comes with extensive example files. It is compiled as a stand-alone program; it does not require Hypercard (although a much smaller ‘stack’ version is included). This program will run in any Macintosh environment running MacOS v7.1 or higher. Faster processor machines (68040 or PowerPC) is suggested for mutual information analysis or the analysis of large sequence alignments.

In another embodiment of the invention, secondary structure analysis is performed by secondary structure prediction. There are a number of algorithms that predict RNA secondary structures based on thermodynamic parameters and energy calculations. Preferably, secondary structure prediction is performed using either M-fold or RNA Structure 2.52. M-fold can be accessed through the world wide web of the Internet at, for example, ibc.wustl.edu/-zuker/ma/form2.cgi or can be downloaded for local use on UNIX platforms. M-fold is also available as a part of GCG package. RNA Structure 2.52 is a windows adaptation of the M-fold algorithm and can be accessed through the world wide web of the Internet at, for example, 128.151.176.70/RNAstructure.html.

In another embodiment of the invention, secondary structure analysis is performed by self complementarity comparison. Preferably, self complementarity comparison is performed using Compare, described above. More preferably, Compare can be modified to expand the pairing matrix to account for G-U or U-G basepairs in addition to the conventional Watson-Crick G-C/C-G or A-U/U-A pairs. Such a modified Compare program (modified Compare) begins by predicting all possible base-pairings within a given sequence. As described above, a small but conserved region is identified based on primary sequence comparison of a series of orthologs. In modified Compare, each of these sequences is compared to its own reverse complement. Allowable base-pairings include Watson-Crick A-U, G-C pairing and non-canonical G-U pairing. An overlay of such self complementarity plots of all available orthologs, and selection for the most repetitive pattern in each, results in a minimal number of possible folded configurations. These overlays can then used in conjunction with additional constraints, including those imposed by energy considerations described above, to deduce the most likely secondary structure.

In another embodiment of the invention, the output of AlignHits is read by a program called RevComp. This program could be reproduced by one skilled in the art. One purpose of this program is to use base pairing rules and ortholog evolution to predict RNA secondary structure. RNA secondary structures are composed of single stranded regions and base paired regions, called stems. Since structure conserved by evolution is searched, the most probable stem for a given alignment of ortholog sequences is the one which could be formed by the most sequences. Possible stem formation or base pairing rules is determined by, for example, analyzing base pairing statistics of stems which have been determined by other techniques such as NMR. The output of RevComp is a sorted list of possible structures, ranked by the percentage of ortholog set member sequences which could form this structure. Because this approach uses a percentage threshold approach, it is insensitive to noise sequences. Noise sequences are those that either not true orthologs, or sequences that made it into the output of AlignHits due to high sequence homology even though they do not represent an example of the structure which is searched. A very similar algorithm is implemented using Visual basic for Applications (VBA) and Microsoft Excel to be run on PCs, to generate the reverse complement matrix view for the given set of sequences.

A result of the secondary structure analysis described above, whether performed by alignment and covariance, self complementarity analysis, secondary structure predictions, such as using. M-fold or otherwise, is the identification of secondary structure in the conserved regions among the target 16S rRNA and the plurality of 16S rRNAs from different taxonomic species. Exemplary secondary structures that may be identified include, but are not limited to, bulges, loops, stems, hairpins, knots, triple interacts, cloverleafs, or helices, or a combination thereof. Alternatively, new secondary structures may be identified.

The present invention is also directed to nucleic acid molecules, such as polynucleotides and oligonucleotides, comprising a molecular interaction site present in 16S rRNA. Nucleic acid molecules include the physical compounds themselves as well as in silico representations of the same. Thus, the nucleic acid molecules are derived from 16S rRNA. The molecular interaction site serves as a binding site for at least one molecule which, when bound to the molecular interaction site, modulates the expression of the 16S rRNA in a cell. The nucleotide sequence of the polynucleotide is selected to provide the secondary structure of the molecular interaction sites described in grater detail in the Examples. The nucleotide sequence of the polynucleotide is preferably the nucleotide sequence of the target 16S rRNAs, described above. Alternatively, the nucleotide sequence is preferably the nucleotide sequence of 16S rRNAs from a plurality of different taxonomic species which also contain the molecular interaction site.

The polynucleotides of the invention comprise the molecular interaction sites of the 16S rRNA. Thus, the polynucleotides of the invention comprise the nucleotide sequences of the molecular interaction sites. In addition, the polynucleotides can comprise up to 50, more preferably up to 40, more preferably up to 30, more preferably up to 20, and most preferably up to 10 additional nucleotides at either the 5′ or 3′, or combination thereof, ends of each polynucleotide. Thus, for example, if a molecular interaction site comprises 25 nucleotides, the polynucleotide can comprise up to 75 nucleotides. The nucleotides that are in addition to those present in the molecular interaction site are selected to preserve the secondary structure of the molecular interaction site. One skilled in the art can select such additional nucleotides so as to conserve the secondary structure. The polynucleotides can comprise either RNA or DNA or can be chimeric RNA/DNA. The polynucleotides can comprise modified bases, sugars and backbones that are well known to the skilled artisan. Further, a single polynucleotide can comprise a plurality of molecular interaction sites. In addition, a plurality of polynucleotides can, together, comprise a single molecular interaction site. Alternatively, when a plurality of polynucleotides together comprise a molecular interaction site, one skilled in the art can attach the polynucleotides to one another, thus, forming a single polynucleotide.

The portion of the polynucleotide comprising the molecular interaction site can comprise one or more deletions, insertions and substitutions. Stems, terminal loops, bulges, internal loops, and dangling regions can comprise one or more deletions, insertions and substitutions. Thus, for example, a terminal loop of a molecular interaction site that consists of ten nucleotides can be modified to contain one or more insertions, deletions or substitutions, thus, resulting in a shortening or lengthening of the stem preceding the terminal loop. In addition, unpaired, dangling nucleotides that are adjacent to, for example, a double-stranded region can be deleted or can be basepaired with the addition of another nucleotide, thus, lengthening the stem. In addition, nucleotide base pairings within a stem can also be substituted, deleted, or inserted. Thus, for example, an A-U basepair within a stem portion of a molecular interaction site can be replaced with a G-C basepair. Further, non-canonical base pairing (e.g., G-A, C-T, G-U, etc.) can also be present within the polynucleotide. Thus, polynucleotides having at least 70%, more preferably 80%, more preferably 90%, more preferably 95%, and most preferably 99% homology with the molecular interaction sites, such as those set forth in the Examples below, are included within the scope of the invention. Percent homology can be determined by, for example, the Gap program (Wisconsin Sequence Analysis Package, Version 8 for Unix, Genetics Computer Group, University Research Park, Madison Wis.), using the default settings, which uses the algorithm of Smith and Waterman (Adv. Appl. Math., 1981, 2, 482-489, which is incorporated herein by reference in its entirety).

The present invention is also directed to the purified and isolated nucleic acid molecules, or polynucleotides, described above, that are present within 16S rRNA. The polynucleotides comprising the molecular interaction site mimic the portion of the 16S rRNA comprising the molecular interaction site.

Polynucleotides, and modifications thereof, are well known to those skilled in the art. The polynucleotides of the invention can be used, for example, as research reagents to detect, for example, naturally occurring molecules that bind the molecular interaction sites. Alternatively, the polynucleotides of the invention can be used to screen, either actually or virtually, small molecules that bind the molecular interaction sites, as described below in greater detail. Virtual generation of compounds and screening thereof for binding to molecular interaction sites is described in, for example, International Publication WO 99/58947, which is incorporated herein by reference in its entirety. The polynucleotides of the invention can also be used as decoys to compete with naturally-occurring molecular interaction sites within a cell for research, diagnostic and therapeutic applications. In particular, the polynucleotides can be used in, for example, therapeutic applications to inhibit bacterial growth. Molecules that bind to the molecular interaction site modulate, either by augmenting or diminishing, the function of 16S rRNA in translation. The polynucleotides can also be used in agricultural, industrial and other applications.

The present invention is also directed to compositions comprising at least one polynucleotide described above. In some embodiments of the invention, two polynucleotides are included within a composition. The compositions of the invention can optionally comprise a carrier. A “carrier” is an acceptable solvent, diluent, suspending agent or any other inert vehicle for delivering one or more nucleic acids to an animal, and are well known to those skilled in the art. The carrier can be a pharmaceutically acceptable carrier. The carrier can be liquid or solid and is selected, with the planned manner of administration in mind, so as to provide for the desired bulk, consistency, etc., when combined with the other components of the composition. Typical pharmaceutical carriers include, but are not limited to, binding agents (e.g., pregelatinised maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrates (e.g., starch, sodium starch glycolate, etc.); or wetting agents (e.g., sodium lauryl sulphate, etc.).

The present invention is also directed to methods of identifying compounds that bind to a. molecular interaction site of 16S rRNA comprising providing a numerical representation of the three-dimensional structure of the molecular interaction site and providing a compound data set comprising numerical representations of the three dimensional structures of a plurality of organic compounds. The numerical representation of the molecular interaction site is then compared with members of the compound data set to generate a hierarchy of organic compounds ranked in accordance with the ability of the organic compounds to form physical interactions with the molecular interaction site.

The present invention is also directed to methods of identifying compounds that bind to a molecular interaction site of 16S rRNA, or a polynucleotide comprising the same. In some embodiments of the invention, compounds that bind to a molecular interaction site of 16S rRNA, or a polynucleotide comprising the same, are identified according to the general methods described in International Publication WO 99/58947, which is incorporated herein by reference in its entirety. Briefly, the methods comprise providing a numerical representation of the three dimensional structure of the molecular interaction site, or a polynucleotide comprising the same, providing a compound data set comprising numerical representations of the three dimensional structures of a plurality of organic compounds, comparing the numerical representation of the molecular interaction site with members of the compound data set to generate a hierarchy of organic compounds which is ranked in accordance with the ability of the organic compounds to form physical interactions with the molecular interaction site.

While there are a number of ways to characterize binding between molecular interaction sites and ligands, such as for example, organic compounds, methodologies are described in International Publications WO 99/58719, WO 99/59061, WO 99/58722, WO 99/45150, WO 99/58474, and WO 99/58947, each of which is assigned to the assignee of the present inventions, and each of which is incorporated by reference herein in their entirety.

In addition, the present invention is also directed to three dimensional representations of the nucleic acid molecules, and compositions comprising the same, described above. The three dimensional structure of a molecular interaction site of 16S rRNA can be manipulated as a numerical representation. The three dimensional representations, i.e., in silico (e.g. in computer-readable form) representations can be generated by methods disclosed in, for example, International Publication WO 99/58947, which is incorporated herein by reference in its entirety. Briefly, the three dimensional structure of a molecular interaction site, preferably of an RNA, can be manipulated as a numerical representation. Computer software that provides one skilled in the art with the ability to design molecules based on the chemistry being performed and on available reaction building blocks is commercially available. Software packages such as, for example, Sybyl/Base (Tripos, St. Louis, Mo.), Insight II (Molecular Simulations, San Diego, Calif.), and Sculpt (MDL Information Systems, San Leandro, Calif.) provide means for computational generation of structures. These software products also provide means for evaluating and comparing computationally generated molecules and their structures. In silico collections of molecular interaction sites can be generated using the software from any of the above-mentioned vendors and others which are or may become available. The three dimensional representations can be used, for example, to dock the molecule(s) to potential therapeutic compounds. Thus, the three dimensional representations can be used in drug screening procedures. Accordingly, the nucleic acid molecules and compositions comprising the same of the present invention include the three dimensional representations of the same.

A set of structural constraints for the molecular interaction site of the 16 S rRNA can be generated from biochemical analyses such as, for example, enzymatic mapping and chemical probes, and from genomics information such as, for example, covariance and sequence conservation. Information such as this can be used to pair bases in the stem or other region of a particular secondary structure. Additional structural hypotheses can be generated for noncanonical base pairing schemes in loop and bulge regions. A Monte Carlo search procedure can sample the possible conformations of the 16 S rRNA consistent with the program constraints and produce three dimensional structures.

Reports of the generation of three dimensional, in silico representations are available from the standpoint of library design, generation, and screening against protein targets. Likewise, some efforts in the area of generating RNA models have been reported in the literature. However, there are no reports on the use of structure-based design approaches to query in silico representations of organic molecules, “small” molecules, polynucleotides or other nucleic acids, with three dimensional, in silico, representations of 16 S rRNA structures. The present invention preferably employs computer software that allows the construction of three dimensional models of 16 S rRNA structure, the construction of three dimensional, in silico representations of a plurality of organic compounds, “small” molecules, polymeric compounds, polynucleotides and other nucleic acids, screening of such in silico representations against 16 S rRNA molecular interaction sites in silico, scoring and identifying the best potential binders from the plurality of compounds, and finally, synthesizing such compounds in a combinatorial fashion and testing them experimentally to identify new ligands for such 16 S rRNA targets.

The molecules that may be screened by using the methods of this invention include, but are not limited to, organic or inorganic, small to large molecular weight individual compounds, and combinatorial mixture or libraries of ligands, inhibitors, agonists, antagonists, substrates, and biopolymers, such as peptides or polynucleotides. Combinatorial mixtures include, but are not limited to, collections of compounds, and libraries of compounds. These mixtures may be generated via combinatorial synthesis of mixtures or via admixture of individual compounds. Collections of compounds include, but are not limited to, sets of individual compounds or sets of mixtures or pools of compounds. These combinatorial libraries may be obtained from synthetic or from natural sources such as, for example to, microbial, plant, marine, viral and animal materials. Combinatorial libraries include at least about twenty compounds and as many as a thousands of individual compounds and potentially even more. When combinatorial libraries are mixtures of compounds these mixtures typically contain from 20 to 5000 compounds preferably from 50 to 1000, more preferably from 50 to 100. Combinations of from 100 to 500 are useful as are mixtures having from 500 to 1000 individual species. Typically, members of combinatorial libraries have molecular weight less than about 10,000 Da, more preferably less than 7,500 Da, and most preferably less than 5000 Da.

A significant advance in the area of virtual screening was the development of a software program called DOCK that allows structure-based database searches to find and identify the interactions of known molecules to a receptor of interest (Kuntz et al., Acc. Chem. Res., 1994, 27, 117; Gschwend and Kuntz, J. Compt.-Aided Mol. Des., 1996, 10, 123). DOCK allows the screening of molecules, whose 3D structures have been generated in silico, but for which no prior knowledge of interactions with the receptor is available. DOCK, therefore, provides a tool to assist in discovering new ligands to a receptor of interest. DOCK can thus be used for docking the compounds prepared according to the methods of the present invention to desired target molecules. Implementation of DOCK is described in, for example, International Publication WO 99/58947, which is incorporated herein by reference in its entirety.

In some embodiments of the invention, an automated computational search algorithm, such as those described above, is used to predict all of the allowed three dimensional molecular interaction site structures from 16S rRNA, which are consistent with the biochemical and genomic constraints specified by the user. Based, for example, on their root-mean-squared deviation values, these structures are clustered into different families. A representative member or members of each family can be subjected to further structural refinement via molecular dynamics with explicit solvent and cations.

Structural enumeration and representation by these software programs is typically done by drawing molecular scaffolds and substituents in two dimensions. Once drawn and stored in the computer, these molecules may be rendered into three dimensional structures using algorithms present within the commercially available software. Preferably, MC-SYM is used to create three dimensional representations of the molecular interaction site. The rendering of two dimensional structures of molecular interaction sites into three dimensional models typically generates a low energy conformation or a collection of low energy conformers of each molecule. The end result of these commercially available programs is the conversion of a 16S rRNA sequence containing a molecular interaction site into families of similar numerical representations of the three dimensional structures of the molecular interaction site. These numerical representations form an ensemble data set.

The three dimensional structures of a plurality of compounds, preferably “small” organic compounds, can be designated as a compound data set comprising numerical representations of the three dimensional structures of the compounds. “Small” molecules in this context refers to non-oligomeric organic compounds. Two dimensional structures of compounds can be converted to three dimensional structures, as described above for the molecular interaction sites, and used for querying against three dimensional structures of the molecular interaction sites. The two dimensional structures of compounds can be generated rapidly using structure rendering algorithms commercially available. The three dimensional representation of the compounds which are polymeric in nature, such as polynucleotides or other nucleic acids structures, may be generated using the literature methods described above. A three dimensional structure of “small” molecules or other compounds can be generated and a low energy conformation can be obtained from a short molecular dynamics minimization. These three dimensional structures can be stored in a relational database. The compounds upon which three dimensional structures are constructed can be proprietary, commercially available, or virtual.

In some embodiments of the invention, a compound data set comprising numerical representations of the three dimensional structure of a plurality of organic compounds is provided by, for example, Converter (MSI, San Diego) from two dimensional compound libraries generated by, for example, a computer program modified from a commercial program. Other suitable databases can be constructed by converting two dimensional structures of chemical compounds into three dimensional structures, as described above. The end result is the conversion of a two dimensional structure of organic compounds into numerical representations of the three dimensional structures of a plurality of organic compounds. These numerical representations are presented as a compound data set.

After both the numerical representations of the three-dimensional structure of the polynucleotides comprising the molecular interaction sites and the compound data set comprising numerical representations of the three dimensional structures of a plurality of organic compounds are obtained, the numerical representations of the molecular interaction sites are compared with members of the compound data set to generate a hierarchy of the organic compounds. The hierarchy is ranked in accordance with the ability of the organic compounds to form physical interactions with the molecular interaction site. Preferably, the comparing is carried out seriatim upon the members of the compound data set. In accordance with some embodiments, the comparison can be performed with a plurality of polynucleotides comprising molecular interaction sites at the same time.

A variety of theoretical and computational methods are known by those skilled in the art to study and optimize the interactions of “small” molecules or organic compounds with biological targets such as nucleic acids. These structure-based drug design tools have been very useful in modelling the interactions of proteins with small molecule ligands and in optimizing these interactions. Typically this type of study has been performed when the structure of the protein receptor was known by querying individual small molecules, one at a time, against this receptor. Usually these small molecules had either been co-crystallized with the receptor, were related to other molecules that had been co-crystallized or were molecules for which some body of knowledge existed concerning their interactions with the receptor. DOCK, as described above, can be used to find and identify molecules that are expected to bind to polynucleotides comprising the molecular interaction sites and, hence, 16S rRNA of interest. DOCK 4.0 is commercially available from the Regents of the University of California. Equivalent programs are also comprehended in the present invention.

The DOCK program has been widely applied to protein targets and the identification of ligands that bind to them. Typically, new classes of molecules that bind to known targets have been identified, and later verified by in vitro experiments. The DOCK software program consists of several modules, including SPHGEN (Kuntz et al., J. Mol. Biol., 1982, 161, 269) and CHEMGRID (Meng et al., J. Comput. Chem., 1992, 13, 505, each of which is incorporated herein by reference in its entirety). SPHGEN generates clusters of overlapping spheres that describe the solvent-accessible surface of the binding pocket within the target receptor. Each cluster represents a possible binding site for small molecules. CHEMGRID precalculates and stores in a grid file the information necessary for force field scoring of the interactions between binding molecule and target 16S rRNA. The scoring function approximates molecular mechanics interaction energies and consists of van der Waals and electrostatic components. DOCK uses the selected cluster of spheres to orient ligands molecules in the targeted site on 16S rRNA. Each molecule within a previously generated three dimensional database is tested in thousands of orientations within the site, and each orientation is evaluated by the scoring function. Only that orientation with the best score for each compound so screened is stored in the output file. Finally, all compounds of the database are ranked in a hierarchy in order of their scores and a collection of the best candidates may then be screened experimentally.

Using DOCK, numerous ligands have been identified for a variety of protein targets. Recent efforts in this area have resulted in reports of the use of DOCK to identify and design small molecule ligands that exhibit binding specificity for nucleic acids such as RNA double helices. While RNA plays a significant role in many diseases such as AIDS, viral and bacterial infections, few studies have been made on small molecules capable of specific RNA binding. Compounds possessing specificity for the RNA double helix, based on the unique geometry of its deep major groove, were identified using the DOCK methodology. Chen et al., Biochemistry, 1997, 36, 11402 and Kuntz et al., Acc. Chem. Res., 1994, 27, 117. Recently, the application of DOCK to the problem of ligand recognition in DNA quadruplexes has been reported. Chen et al., Proc. Natl. Acad. Sci., 1996, 93, 2635.

Preferably, individual compounds are designated as mol files, for example, and combined into a collection of in silico representations using an appropriate chemical structure program or equivalent software. These two dimensional mol files are exported and converted into three dimensional structures using commercial software such as Converter (Molecular Simulations Inc., San Diego) or equivalent software, as described above. Atom types suitable for use with a docking program such as DOCK or QXP are assigned to all atoms in the three dimensional mol file using software such as, for example, Babel, or with other equivalent software.

A low-energy conformation of each molecule is generated with software such as Discover (MSI, San Diego). An orientation search is performed by bringing each compound of the plurality of compounds into proximity with the molecular interaction site in many orientations using DOCK or QXP. A contact score is determined for each orientation, and the optimum orientation of the compound is subsequently used. Alternatively, the conformation of the compound can be determined from a template conformation of the scaffold determined previously.

The interaction of a plurality of compounds and molecular interaction sites is examined by comparing the numerical representations of the molecular interaction sites with members of the compound data set. Preferably, a plurality of compounds such as those generated by a computer program or otherwise, is compared to the molecular interaction site and undergoes random “motions” among the dihedral bonds of the compounds. Preferably about 20,000 to 100,000 compounds are compared to at least one molecular interaction site. Typically, 20,000 compounds are compared to about five molecular interaction sites and scored. Individual conformations of the three dimensional structures are placed at the target site in many orientations. Moreover, during execution of the DOCK program, the compounds and molecular interaction sites are allowed to be “flexible” such that the optimum hydrogen bonding, electrostatic, and van der Waals contacts can be realized. The energy of the interaction is calculated and stored for 10-15 possible orientations of the compounds and molecular interaction sites. QXP methodology allows true flexibility in both the ligand and target and is presently preferred.

The relative weights of each energy contribution are updated constantly to insure that the calculated binding scores for all compounds reflect the experimental binding data. The binding energy for each orientation is scored on the basis of hydrogen bonding, van der Waals contacts, electrostatics, solvation/desolvation, and the quality of the fit. The lowest-energy van der Waals, dipolar, and hydrogen bonding interactions between the compound and the molecular interaction site are determined, and summed. In some embodiments, these parameters can be adjusted according to the results obtained empirically. The binding energies for each molecule against the target are output to a relational database. The relational database contains a hierarchy of the compounds ranked in accordance with the ability of the compounds to form physical interactions with the molecular interaction site. The higher ranked compounds are better able to form physical interactions with the molecular interaction site.

In another embodiment, the highest ranking, i.e., the best fitting compounds, are selected for synthesis. In some embodiments of the invention, those compounds which are likely to have desired binding characteristics based on binding data are selected for synthesis. Preferably the highest ranking 5% are selected for synthesis. More preferably, the highest ranking 10% are selected for syntheses. Even more preferably, the highest ranking 20% are selected for synthesis. The synthesis of the selected compounds can be automated using a parallel array synthesizer or prepared using solution-phase or other solid-phase methods and instruments. In addition, the interaction of the highly ranked compounds with the nucleic acid containing the molecular interaction site is assessed as described below.

The interaction of the highly ranked organic compounds with the polynucleotide comprising the 16S rRNA molecular interaction site can be assessed by numerous methods known to those skilled in the art. For example, the highest ranking compounds can be tested for activity in high-throughput (HTS) functional and cellular screens. HTS assays can be determined by scintillation proximity, precipitation, luminescence-based formats, filtration based assays, colorometric assays, and the like. Lead compounds can then be scaled up and tested in animal models for activity and toxicity. The assessment preferably comprises mass spectrometry of a mixture of the 16S rRNA polynucleotide and at least one of the compounds or a functional bioassay.

The interaction of a plurality of compounds with a putative molecular interaction site of the 16S rRNA can be assessed by numerous methods known to those skilled in the art. The plurality of compounds can be organic or inorganic compounds, for example, be a library of known compounds, a combinatorial library of compounds, or a mixture of unknown compounds. For example, the compounds can be tested for activity in high-throughput (HTS) functional and cellular screens. HTS assays can be determined by scintillation proximity, precipitation, luminescence-based formats, filtration based assays, colorometric assays, and the like. Lead compounds which have been identified as capable of interacting with the 16S rRNA putative molecular interaction site can then be scaled up and tested in animal models for activity and toxicity. The assessment preferably comprises mass spectrometry of a mixture of polynucleotides comprising the 16S rRNA putative molecular interaction site and at least one of the compounds or a functional bioassay.

Certain evaluation techniques employing mass spectroscopy are disclosed in International Publication WO 99/45150, which is incorporated herein by reference in its entirety, as exemplary of certain useful and mass spectrometric techniques for use herewith. It is to be specifically understood, however, that it is not essential that these particular mass spectrometric techniques be employed in order to perform the present invention. Rather, any evaluative technique may be undertaken so long as the objectives of the present invention are maintained.

In some embodiments of the invention, the highest ranking 20% of compounds from the hierarchy generated using the DOCK program or QXP are used to generate a further data set of three dimensional representations of organic compounds comprising compounds which are chemically related to the compounds ranking high in the hierarchy. Although the best fitting compounds are likely to be in the highest ranking 1%, additional compounds, up to about 20%, are selected for a second comparison so as to provide diversity (ring size, chain length, functional groups). This process insures that small errors in the molecular interaction sites are not propagated into the compound identification process. The resulting structure/score data from the highest ranking 20%, for example, is studied mathematically (clustered) to find trends or features within the compounds which enhance binding. The compounds are clustered into different groups. Chemical synthesis and screening of the compounds, described above, allows the computed DOCK or QXP scores to be correlated with the actual binding data. After the compounds have been prepared and screened, the predicted binding energy and the observed Kd values are correlated for each compound.

The results are used to develop a predictive scoring scheme, which weighs various factors (steric, electrostatic) appropriately. The above strategy allows rapid evaluation of a number of scaffolds with varying sizes and shapes of different functional groups for the high ranked compounds. In this manner, a further data set of representations of organic compounds comprising compounds which are chemically related to the organic compounds which rank high in the hierarchy can be compared to the numerical representations of the molecular interaction site to determine a further hierarchy ranked in accordance with the ability of the organic compounds to form physical interactions with the molecular interaction site. In this manner, the further data set of representations of the three dimensional structures of compound which are related to the compounds ranked high in the hierarchy are produced and have, in effect, been optimized by correlating actual binding with virtual binding. The entire cycle can be iterated as desired until the desired number of compounds highest in the hierarchy are produced.

Compounds which have been determined to have affinity and specificity for a target biomolecule, especially a target 16S rRNA or which otherwise have been shown to be able to bind to the target 16S rRNA to effect modulation thereof, can, in accordance with some embodiments of this invention, be tagged or labelled in a detectable fashion. Such labelling may include all of the labelling forms known to persons of skill in the art such as fluorophore, radiolabel, enzymatic label and many other forms. Such labelling or tagging facilitates detection of molecular interaction sites and permits facile mapping of chromosomes and other useful processes.

Some of the preferred embodiments of the invention described above are outlined below and include, but are not limited to, the following embodiments. Thus, the following examples are meant to be exemplary of some of the invention and are not meant to be limiting. As those skilled in the art will appreciate, numerous changes and modifications may be made to the embodiments of the invention without departing from the spirit of the invention. It is intended that all such variations fall within the scope of the invention.

### EXAMPLES

### Example 1

### Selection of 16S rRNA

To illustrate the strategy for identifying molecular interaction sites for small molecules, the 16S rRNA was used. The structure of the 16S rRNA has been determined using NMR spectroscopy. Konings et al., RNA, 1995, 1, 559-574, which is incorporated herein by reference. The 16S rRNA is an RNA of approximately 1540 nucleotides that folds, generally into three domains, a 5′ domain, a 3′ domain, and a central domain.

### Example 2

### Molecular Interaction Sites in Consensus 16S rRNA

Numerous molecular interaction sites have been discovered within 16S rRNA. Consensus site 1 comprises a region of RNA comprising a first and second polynucleotide. The first polynucleotide comprises from about four nucleotides to about eleven nucleotides, wherein portions of the polynucleotide form a double-stranded RNA having the following features (5′ to 3′): a first side of a stem comprising from about three nucleotides to about-nine nucleotides wherein a bulge comprising from about one nucleotide to about two nucleotides is present between the third and fourth nucleotides of the first side of the stem. The second polynucleotide comprises from about three nucleotides to about nine nucleotides, wherein portions of the polynucleotide form a double-stranded RNA having the following features (5′ to 3′): a second side of the stem comprising from about three nucleotides to about nine nucleotides.

In regard to consensus site 1, the first polynucleotide preferably comprises seven nucleotides, wherein portions of the polynucleotide form a double-stranded RNA having the following features (5′ to 3′): a first side of a stem comprising six nucleotides wherein a bulge comprising one nucleotide is present between the third and fourth nucleotides of the first side of the stem. Preferably, the first polynucleotide comprises the sequence 5′-nnngacc-3′ (bolded nucleotides indicate preferred basepairing; n is any nucleotide). The second polynucleotide preferably comprises six nucleotides, wherein portions of the polynucleotide form a double-stranded RNA having the following features (5′ to 3′): a second side of the stem comprising six nucleotides. Preferably, the second polynucleotide comprises 5′-guunnn-3′ (bolded nucleotides indicate preferred basepairing; n is any nucleotide). Consensus site 1 is depicted in FIG. 1.

Consensus site 2 comprises a region of RNA comprising a first and second polynucleotide. The first polynucleotide comprises from about four nucleotides to about twelve nucleotides, wherein portions of the polynucleotide form a double-stranded RNA having the following features (5′ to 3′): a first side of a stem comprising from about two nucleotides to about six nucleotides, and a dangling region comprising from about two nucleotides to about six nucleotides. The second polynucleotide comprises from about four nucleotides to about eleven nucleotides, wherein portions of the polynucleotide form a double-stranded RNA having the following features (5′ to 3′): a second side of the stem comprising from about two nucleotides to about six nucleotides wherein a bulge comprising from about one nucleotide to about two nucleotides is present in the second side of the stem and wherein a bulge comprising from about one nucleotide to about three nucleotides is present in the second side of the stem.

In regard to consensus site 2, the first -polynucleotide preferably comprises eight nucleotides, wherein portions of the polynucleotide form a double-stranded RNA having the following features (5′ to 3′): a first side of a stem comprising four nucleotides, and a dangling region comprising four nucleotides. Preferably, the first polynucleotide comprises the sequence 5′-unnggaau-3′ (bolded nucleotides indicate preferred basepairing; n is any nucleotide). The second polynucleotide preferably comprises seven nucleotides, wherein portions of the polynucleotide form a double-stranded RNA having the following features (5′ to 3′): a second side of the stem comprising four nucleotides wherein a bulge comprising one nucleotide is present between the first and second nucleotides of the second side of the stem and wherein a bulge comprising two nucleotides is present between the second and third nucleotides of the second side of the stem. Preferably, the second polynucleotide comprises the sequence 5′-cnunana-3′ (bolded nucleotides indicate preferred basepairing; n is any nucleotide). Consensus site 2 is depicted in FIG. 1.

Consensus site 3 comprises a region of RNA comprising a first and second polynucleotide. The first polynucleotide comprises from about four nucleotides to about twelve nucleotides, wherein portions of the polynucleotide form a double-stranded RNA having the following features (5′ to 3′): a dangling region comprising from about one nucleotide to about three nucleotides and a first side of a stem comprising from about three nucleotides to about nine nucleotides. The second polynucleotide comprises from about four nucleotides to about eleven nucleotides, wherein portions of the polynucleotide form a double-stranded RNA having the following features (5′ to 3′): a second side of the stem comprising from about three nucleotides to about nine nucleotides wherein a bulge comprising from about one nucleotide to about two nucleotides is present in the second side of the stem.

In regard to consensus site 3, the first polynucleotide preferably comprises eight nucleotides, wherein portions of the polynucleotide form a double-stranded RNA having the following features (5′ to 3′): a dangling region comprising two nucleotides and a first side of a stem comprising six nucleotides. Preferably, the first polynucleotide comprises the sequence 5′-cagcagun-3′ (bolded nucleotides indicate preferred basepairing; n is any nucleotide). The second polynucleotide preferably comprises seven nucleotides, wherein portions of the polynucleotide form a double-stranded RNA having the following features (5′ to 3′): a second side of the stem comprising six nucleotides wherein a bulge comprising one nucleotide is present between the third and fourth nucleotides of the second side of the stem. Preferably, the second polynucleotide comprises the sequence 5′-cguacan-3′ (bolded nucleotides indicate preferred basepairing; n is any nucleotide). Consensus site 3 is depicted in FIG. 1.

Consensus site 4 comprises a region of RNA comprising a first and second polynucleotide. The first polynucleotide comprises from about four nucleotides to about twelve nucleotides, wherein portions of the polynucleotide form a double-stranded RNA having the following features (5′ to 3′): a first side of a stem comprising from about three nucleotides to about nine nucleotides wherein a bulge comprising from about one nucleotide to about three nucleotides is present in the first side of the stem. The second polynucleotide comprises from about three nucleotides to about nine nucleotides, wherein portions of the polynucleotide form a double-stranded RNA having the following features (5′ to 3′): a second side of the stem comprising from about three nucleotides to about nine nucleotides.

In regard to consensus site 4, the first polynucleotide preferably comprises eight nucleotides, wherein portions of the polynucleotide form a double-stranded RNA having the following features (5′ to 3′): a first side of a stem comprising six nucleotides wherein a bulge comprising two nucleotides is present between the third and fourth nucleotides of the first side of the stem. Preferably, the first polynucleotide comprises the sequence 5′-gucgancg-3′ (bolded nucleotides indicate preferred basepairing; n is any nucleotide). The second polynucleotide preferably comprises six nucleotides, wherein portions of the polynucleotide form a double-stranded RNA having the following features (5′ to 3′): a second side of the stem comprising six nucleotides. Preferably, the second polynucleotide comprises the sequence 5′-agnggc-3′ (bolded nucleotides indicate preferred basepairing; n is any nucleotide). Consensus site 4 is depicted in FIG. 1. Consensus site 5 comprises a region of RNA comprising a polynucleotide comprising from about four nucleotides to about one hundred sixty seven nucleotides, wherein portions of the polynucleotide form a double-stranded RNA having the following features (5′ to 3′): a first side of a stem comprising from about two nucleotides to about five nucleotides, an optional terminal loop comprising from about one nucleotide to about one hundred fifty seven nucleotides, and a second side of the stem comprising from about two nucleotides to about five nucleotides.

In regard to consensus site 5, the polynucleotide preferably comprises from six to one hundred sixty three nucleotides, wherein portions of the polynucleotide form a double-stranded RNA having the following features (5′ to 3′): a first side of a stem comprising three nucleotides, an optional terminal loop comprising up to 157 nucleotides, and a second side of the stem comprising three nucleotides. Preferably, the polynucleotide comprises the sequence

| | |
|---|---|
| 5′-ncgagn-3′, | |
| 5′-ncgnagn-3′, | |
| 5′-ncgnnagn-3′, | |
| 5′-ncgnnnagn-3′, | |
| 5′-ncgnnnnagn-3′, | (SEQ ID NO:1) |
| 5′-ncgnnnnnagn-3′, | (SEQ ID NO:2) |
| 5′-ncgnnnnnnagn-3′, | (SEQ ID NO:3) |
| 5′-ncgnnnnnnnagn-3′, | (SEQ ID NO:4) |
| 5′-ncgnnnnnnnnagn-3′, | (SEQ ID NO:5) |
| 5′-ncgnnnnnnnnnagn-3′, | (SEQ ID NO:6) |
| 5′-ncgnnnnnnnnnnagn-3′, | (SEQ ID NO:7) |
| 5′-ncgnnnnnnnnnnnagn-3′, | (SEQ ID NO:8) |
| 5′-ncgnnnnnnnnnnnnagn-3′, | (SEQ ID NO:9) |
| 5′-ncgnnnnnnnnnnnnnagn-3′, | (SEQ ID NO:10) |
| 5′-ncgnnnnnnnnnnnnnnagn-3′, | (SEQ ID NO:11) |
| 5′-ncgnnnnnnnnnnnnnnnagn-3′, | (SEQ ID NO:12) |
| 5′-ncgnnnnnnnnnnnnnnnnagn-3′, | (SEQ ID NO:13) |
| 5′-ncgnnnnnnnnnnnnnnnnnagn-3′, | (SEQ ID NO:14) |
| 5′-ncgnnnnnnnnnnnnnnnnnnagn-3′, | (SEQ ID NO:15) |
| 5′-ncgnnnnnnnnnnnnnnnnnnnagn-3′, | (SEQ ID NO:16) |
| 5′-ncgnnnnnnnnnnnnnnnnnnnnagn-3′, | (SEQ ID NO:17) |
| 5′-ncgnnnnnnnnnnnnnnnnnnnnnagn-3′, | (SEQ ID NO:18) |
| 5′-ncgnnnnnnnnnnnnnnnnnnnnnnagn-3′, | (SEQ ID NO:19) |
| 5′-ncgnnnnnnnnnnnnnnnnnnnnnnnagn-3′, | (SEQ ID NO:20) |
| 5′-ncgnnnnnnnnnnnnnnnnnnnnnnnnagn-3′, | (SEQ ID NO:21) |
| 5′-ncgnnnnnnnnnnnnnnnnnnnnnnnnnagn-3′, | (SEQ ID NO:22) |
| 5′-ncgnnnnnnnnnnnnnnnnnnnnnnnnnnagn-3′, | (SEQ ID NO:23) |
| 5′-ncgnnnnnnnnnnnnnnnnnnnnnnnnnnnagn-3′, | (SEQ ID NO:24) |
| 5′-ncgnnnnnnnnnnnnnnnnnnnnnnnnnnnnagn-3′, | (SEQ ID NO:25) |
| 5′-ncgnnnnnnnnnnnnnnnnnnnnnnnnnnnnnagn-3′, | (SEQ ID NO:26) |
| 5′-ncgnnnnnnnnnnnnnnnnnnnnnnnnnnnnnnagn-3′, | (SEQ ID NO:27) |
| 5′-ncgnnnnnnnnnnnnnnnnnnnnnnnnnnnnnnnagn-3′, | (SEQ ID NO:28) |
| 5′-ncgnnnnnnnnnnnnnnnnnnnnnnnnnnnnnnnnagn-3′, | (SEQ ID NO:29) |
| 5′-ncgnnnnnnnnnnnnnnnnnnnnnnnnnnnnnnnnnagn-3′, | (SEQ ID NO:30) |
| 5′-ncgnnnnnnnnnnnnnnnnnnnnnnnnnnnnnnnnnnagn-3′, | (SEQ ID NO:31) |
| 5′-ncgnnnnnnnnnnnnnnnnnnnnnnnnnnnnnnnnnnnagn-3′, | (SEQ ID NO:32) |
| 5′-ncgnnnnnnnnnnnnnnnnnnnnnnnnnnnnnnnnnnnnagn-3′, | (SEQ ID NO:33) |
| 5′-ncgnnnnnnnnnnnnnnnnnnnnnnnnnnnnnnnnnnnnnagn-3′, | (SEQ ID NO:34) |
| 5′-ncgnnnnnnnnnnnnnnnnnnnnnnnnnnnnnnnnnnnnnnagn-3′, | (SEQ ID NO:35) |
| 5′-ncgnnnnnnnnnnnnnnnnnnnnnnnnnnnnnnnnnnnnnnnagn-3′, | (SEQ ID NO:36) |
| 5′-ncgnnnnnnnnnnnnnnnnnnnnnnnnnnnnnnnnnnnnnnnnagn-3′, | (SEQ ID NO:37) |
| 5′-ncgnnnnnnnnnnnnnnnnnnnnnnnnnnnnnnnnnnnnnnnnnagn-3′, | (SEQ ID NO:38) |
| 5′-ncgnnnnnnnnnnnnnnnnnnnnnnnnnnnnnnnnnnnnnnnnnnagn-3′, | (SEQ ID NO:39) |
| 5′-ncgnnnnnnnnnnnnnnnnnnnnnnnnnnnnnnnnnnnnnnnnnnnagn-3′, | (SEQ ID NO:40) |
| 5′-ncgnnnnnnnnnnnnnnnnnnnnnnnnnnnnnnnnnnnnnnnnnnnnagn-3′, | (SEQ ID NO:41) |
| 5′-ncgnnnnnnnnnnnnnnnnnnnnnnnnnnnnnnnnnnnnnnnnnnnnnagn-3′, | (SEQ ID NO:42) |
| 5′-ncgnnnnnnnnnnnnnnnnnnnnnnnnnnnnnnnnnnnnnnnnnnnnnnagn-3′, | (SEQ ID NO:43) |
| 5′-ncgnnnnnnnnnnnnnnnnnnnnnnnnnnnnnnnnnnnnnnnnnnnnnnnagn-3′, | (SEQ ID NO:44) |
| 5′-ncgnnnnnnnnnnnnnnnnnnnnnnnnnnnnnnnnnnnnnnnnnnnnnnnnagn-3′, | (SEQ ID NO:45) |
| 5′-ncgnnnnnnnnnnnnnnnnnnnnnnnnnnnnnnnnnnnnnnnnnnnnnnnnnagn-3′, | (SEQ ID NO:46) |
| 5′-ncgnnnnnnnnnnnnnnnnnnnnnnnnnnnnnnnnnnnnnnnnnnnnnnnnnnagn-3′, | (SEQ ID NO:47) |
| 5′-ncgnnnnnnnnnnnnnnnnnnnnnnnnnnnnnnnnnnnnnnnnnnnnnnnnnnnagn-3′, | (SEQ ID NO:48) |
| 5′-ncgnnnnnnnnnnnnnnnnnnnnnnnnnnnnnnnnnnnnnnnnnnnnnnnnnnnnagn-3′, | (SEQ ID NO:49) |
| 5′-ncgnnnnnnnnnnnnnnnnnnnnnnnnnnnnnnnnnnnnnnnnnnnnnnnnnnnnnagn-3′, | (SEQ ID NO:50) |
| 5′-ncgnnnnnnnnnnnnnnnnnnnnnnnnnnnnnnnnnnnnnnnnnnnnnnnnnnnnnnagn-3′, | (SEQ ID NO:51) |
| 5′-ncgnnnnnnnnnnnnnnnnnnnnnnnnnnnnnnnnnnnnnnnnnnnnnnnnnnnnnnnagn-3′, | (SEQ ID NO:52) |
| 5′-ncgnnnnnnnnnnnnnnnnnnnnnnnnnnnnnnnnnnnnnnnnnnnnnnnnnnnnnnnnagn-3′, | (SEQ ID NO:53) |
| 5′-ncgnnnnnnnnnnnnnnnnnnnnnnnnnnnnnnnnnnnnnnnnnnnnnnnnnnnnnnnnnagn-3′, | (SEQ ID NO:54) |
| 5′-ncgnnnnnnnnnnnnnnnnnnnnnnnnnnnnnnnnnnnnnnnnnnnnnnnnnnnnnnnnnnagn-3′, | (SEQ ID NO:55) |
| 5′-ncgnnnnnnnnnnnnnnnnnnnnnnnnnnnnnnnnnnnnnnnnnnnnnnnnnnnnnnnnnnnagn-3′, | (SEQ ID NO:56) |
| 5′-ncgnnnnnnnnnnnnnnnnnnnnnnnnnnnnnnnnnnnnnnnnnnnnnnnnnnnnnnnnnnnnagn-3′, | (SEQ ID NO:57) |
| 5′-ncgnnnnnnnnnnnnnnnnnnnnnnnnnnnnnnnnnnnnnnnnnnnnnnnnnnnnnnnnnnnnnagn-3′, | (SEQ ID NO:58) |
| 5′-ncgnnnnnnnnnnnnnnnnnnnnnnnnnnnnnnnnnnnnnnnnnnnnnnnnnnnnnnnnnnnnnnagn-3′, | (SEQ ID NO:59) |
| 5′-ncgnnnnnnnnnnnnnnnnnnnnnnnnnnnnnnnnnnnnnnnnnnnnnnnnnnnnnnnnnnnnnnnagn-3′, | (SEQ ID NO:60) |
| 5′-ncgnnnnnnnnnnnnnnnnnnnnnnnnnnnnnnnnnnnnnnnnnnnnnnnnnnnnnnnnnnnnnnnnagn-3′, | (SEQ ID NO:61) |
| 5′-ncgnnnnnnnnnnnnnnnnnnnnnnnnnnnnnnnnnnnnnnnnnnnnnnnnnnnnnnnnnnnnnnnnnagn-3′, | (SEQ ID NO:62) |
| 5′-ncgnnnnnnnnnnnnnnnnnnnnnnnnnnnnnnnnnnnnnnnnnnnnnnnnnnnnnnnnnnnnnnnnnnagn-3′, | (SEQ ID NO:63) |
| 5′-ncgnnnnnnnnnnnnnnnnnnnnnnnnnnnnnnnnnnnnnnnnnnnnnnnnnnnnnnnnnnnnnnnnnnnagn-3′, | (SEQ ID NO:64) |
| 5′-ncgnnnnnnnnnnnnnnnnnnnnnnnnnnnnnnnnnnnnnnnnnnnnnnnnnnnnnnnnnnnnnnnnnnnnagn-3′, | (SEQ ID NO:65) |
| 5′-ncgnnnnnnnnnnnnnnnnnnnnnnnnnnnnnnnnnnnnnnnnnnnnnnnnnnnnnnnnnnnnnnnnnnnnnagn-3′, | (SEQ ID NO:66) |
| 5′-ncgnnnnnnnnnnnnnnnnnnnnnnnnnnnnnnnnnnnnnnnnnnnnnnnnnnnnnnnnnnnnnnnnnnnnnnagn-3′, | (SEQ ID NO:67) |
| 5′-ncgnnnnnnnnnnnnnnnnnnnnnnnnnnnnnnnnnnnnnnnnnnnnnnnnnnnnnnnnnnnnnnnnnnnnnnnagn-3′, | (SEQ ID NO:68) |
| 5′-ncgnnnnnnnnnnnnnnnnnnnnnnnnnnnnnnnnnnnnnnnnnnnnnnnnnnnnnnnnnnnnnnnnnnnnnnnnagn-3′, | (SEQ ID NO:69) |
| 5′-ncgnnnnnnnnnnnnnnnnnnnnnnnnnnnnnnnnnnnnnnnnnnnnnnnnnnnnnnnnnnnnnnnnnnnnnnnnnagn-3′, | (SEQ ID NO:70) |
| 5′-ncgnnnnnnnnnnnnnnnnnnnnnnnnnnnnnnnnnnnnnnnnnnnnnnnnnnnnnnnnnnnnnnnnnnnnnnnnnnag | (SEQ ID NO:71) |
| n-3′, | |
| 5′-ncgnnnnnnnnnnnnnnnnnnnnnnnnnnnnnnnnnnnnnnnnnnnnnnnnnnnnnnnnnnnnnnnnnnnnnnnnnnna | (SEQ ID NO:72) |
| gn-3′, | |
| 5′-ncgnnnnnnnnnnnnnnnnnnnnnnnnnnnnnnnnnnnnnnnnnnnnnnnnnnnnnnnnnnnnnnnnnnnnnnnnnnnn | (SEQ ID NO:73) |
| agn-3′, | |
| 5′-ncgnnnnnnnnnnnnnnnnnnnnnnnnnnnnnnnnnnnnnnnnnnnnnnnnnnnnnnnnnnnnnnnnnnnnnnnnnnnn | (SEQ ID NO:74) |
| nagn-3′, | |
| 5′-ncgnnnnnnnnnnnnnnnnnnnnnnnnnnnnnnnnnnnnnnnnnnnnnnnnnnnnnnnnnnnnnnnnnnnnnnnnnnnn | (SEQ ID NO:75) |
| nnagn-3′, | |
| 5′-ncgnnnnnnnnnnnnnnnnnnnnnnnnnnnnnnnnnnnnnnnnnnnnnnnnnnnnnnnnnnnnnnnnnnnnnnnnnnnn | (SEQ ID NO:76) |
| nnnagn-3′, | |
| 5′-ncgnnnnnnnnnnnnnnnnnnnnnnnnnnnnnnnnnnnnnnnnnnnnnnnnnnnnnnnnnnnnnnnnnnnnnnnnnnnn | (SEQ ID NO:77) |
| nnnnagn-3′, | |
| 5′-ncgnnnnnnnnnnnnnnnnnnnnnnnnnnnnnnnnnnnnnnnnnnnnnnnnnnnnnnnnnnnnnnnnnnnnnnnnnnnn | (SEQ ID NO:78) |
| nnnnnagn-3′, | |
| 5′-ncgnnnnnnnnnnnnnnnnnnnnnnnnnnnnnnnnnnnnnnnnnnnnnnnnnnnnnnnnnnnnnnnnnnnnnnnnnnnn | (SEQ ID NO:79) |
| nnnnnnagn-3′, | |
| 5′-ncgnnnnnnnnnnnnnnnnnnnnnnnnnnnnnnnnnnnnnnnnnnnnnnnnnnnnnnnnnnnnnnnnnnnnnnnnnnnn | (SEQ ID NO:80) |
| nnnnnnnagn-3′, | |
| 5′-ncgnnnnnnnnnnnnnnnnnnnnnnnnnnnnnnnnnnnnnnnnnnnnnnnnnnnnnnnnnnnnnnnnnnnnnnnnnnnn | (SEQ ID NO:81) |
| nnnnnnnnagn-3′, | |
| 5′-ncgnnnnnnnnnnnnnnnnnnnnnnnnnnnnnnnnnnnnnnnnnnnnnnnnnnnnnnnnnnnnnnnnnnnnnnnnnnnn | (SEQ ID NO:82) |
| nnnnnnnnnagn-3′, | |
| 5′-ncgnnnnnnnnnnnnnnnnnnnnnnnnnnnnnnnnnnnnnnnnnnnnnnnnnnnnnnnnnnnnnnnnnnnnnnnnnnnn | (SEQ ID NO:83) |
| nnnnnnnnnnagn-3′, | |
| 5′-ncgnnnnnnnnnnnnnnnnnnnnnnnnnnnnnnnnnnnnnnnnnnnnnnnnnnnnnnnnnnnnnnnnnnnnnnnnnnnn | (SEQ ID NO:84) |
| nnnnnnnnnnnagn-3′, | |
| 5′-ncgnnnnnnnnnnnnnnnnnnnnnnnnnnnnnnnnnnnnnnnnnnnnnnnnnnnnnnnnnnnnnnnnnnnnnnnnnnnn | (SEQ ID NO:85) |
| nnnnnnnnnnnnagn-3′, | |
| 5′-ncgnnnnnnnnnnnnnnnnnnnnnnnnnnnnnnnnnnnnnnnnnnnnnnnnnnnnnnnnnnnnnnnnnnnnnnnnnnnn | (SEQ ID NO:86) |
| nnnnnnnnnnnnnagn-3′, | |
| 5′-ncgnnnnnnnnnnnnnnnnnnnnnnnnnnnnnnnnnnnnnnnnnnnnnnnnnnnnnnnnnnnnnnnnnnnnnnnnnnnn | (SEQ ID NO:87) |
| nnnnnnnnnnnnnnagn-3′, | |
| 5′-ncgnnnnnnnnnnnnnnnnnnnnnnnnnnnnnnnnnnnnnnnnnnnnnnnnnnnnnnnnnnnnnnnnnnnnnnnnnnnn | (SEQ ID NO:88) |
| nnnnnnnnnnnnnnnagn-3′, | |
| 5′-ncgnnnnnnnnnnnnnnnnnnnnnnnnnnnnnnnnnnnnnnnnnnnnnnnnnnnnnnnnnnnnnnnnnnnnnnnnnnnn | (SEQ ID NO:89) |
| nnnnnnnnnnnnnnnnagn-3′, | |
| 5′-ncgnnnnnnnnnnnnnnnnnnnnnnnnnnnnnnnnnnnnnnnnnnnnnnnnnnnnnnnnnnnnnnnnnnnnnnnnnnnn | (SEQ ID NO:90) |
| nnnnnnnnnnnnnnnnnagn-3′, | |
| 5′-ncgnnnnnnnnnnnnnnnnnnnnnnnnnnnnnnnnnnnnnnnnnnnnnnnnnnnnnnnnnnnnnnnnnnnnnnnnnnnn | (SEQ ID NO:91) |
| nnnnnnnnnnnnnnnnnnagn-3′, | |
| 5′-ncgnnnnnnnnnnnnnnnnnnnnnnnnnnnnnnnnnnnnnnnnnnnnnnnnnnnnnnnnnnnnnnnnnnnnnnnnnnnn | (SEQ ID NO:92) |
| nnnnnnnnnnnnnnnnnnnagn-3′, | |
| 5′-ncgnnnnnnnnnnnnnnnnnnnnnnnnnnnnnnnnnnnnnnnnnnnnnnnnnnnnnnnnnnnnnnnnnnnnnnnnnnnn | (SEQ ID NO:93) |
| nnnnnnnnnnnnnnnnnnnnagn-3′, | |
| 5′-ncgnnnnnnnnnnnnnnnnnnnnnnnnnnnnnnnnnnnnnnnnnnnnnnnnnnnnnnnnnnnnnnnnnnnnnnnnnnnn | (SEQ ID NO:94) |
| nnnnnnnnnnnnnnnnnnnnnagn-3′, | |
| 5′-ncgnnnnnnnnnnnnnnnnnnnnnnnnnnnnnnnnnnnnnnnnnnnnnnnnnnnnnnnnnnnnnnnnnnnnnnnnnnnn | (SEQ ID NO:95) |
| nnnnnnnnnnnnnnnnnnnnnnagn-3′, | |
| 5′-ncgnnnnnnnnnnnnnnnnnnnnnnnnnnnnnnnnnnnnnnnnnnnnnnnnnnnnnnnnnnnnnnnnnnnnnnnnnnnn | (SEQ ID NO:96) |
| nnnnnnnnnnnnnnnnnnnnnnnagn-3′, | |
| 5′-ncgnnnnnnnnnnnnnnnnnnnnnnnnnnnnnnnnnnnnnnnnnnnnnnnnnnnnnnnnnnnnnnnnnnnnnnnnnnnn | (SEQ ID NO:97) |
| nnnnnnnnnnnnnnnnnnnnnnnnagn-3′, | |
| 5′-ncgnnnnnnnnnnnnnnnnnnnnnnnnnnnnnnnnnnnnnnnnnnnnnnnnnnnnnnnnnnnnnnnnnnnnnnnnnnnn | (SEQ ID NO:98) |
| nnnnnnnnnnnnnnnnnnnnnnnnnagn-3′, | |
| 5′-ncgnnnnnnnnnnnnnnnnnnnnnnnnnnnnnnnnnnnnnnnnnnnnnnnnnnnnnnnnnnnnnnnnnnnnnnnnnnnn | (SEQ ID NO:99) |
| nnnnnnnnnnnnnnnnnnnnnnnnnnagn-3′, | |
| 5′-ncgnnnnnnnnnnnnnnnnnnnnnnnnnnnnnnnnnnnnnnnnnnnnnnnnnnnnnnnnnnnnnnnnnnnnnnnnnnnn | (SEQ ID NO:100) |
| nnnnnnnnnnnnnnnnnnnnnnnnnnnagn-3′, | |
| 5′-ncgnnnnnnnnnnnnnnnnnnnnnnnnnnnnnnnnnnnnnnnnnnnnnnnnnnnnnnnnnnnnnnnnnnnnnnnnnnnn | (SEQ ID NO:101) |
| nnnnnnnnnnnnnnnnnnnnnnnnnnnnagn-3′, | |
| 5′-ncgnnnnnnnnnnnnnnnnnnnnnnnnnnnnnnnnnnnnnnnnnnnnnnnnnnnnnnnnnnnnnnnnnnnnnnnnnnnn | (SEQ ID NO:102) |
| nnnnnnnnnnnnnnnnnnnnnnnnnnnnnagn-3′, | |
| 5′-ncgnnnnnnnnnnnnnnnnnnnnnnnnnnnnnnnnnnnnnnnnnnnnnnnnnnnnnnnnnnnnnnnnnnnnnnnnnnnn | (SEQ ID NO:103) |
| nnnnnnnnnnnnnnnnnnnnnnnnnnnnnnagn-3′, | |
| 5′-ncgnnnnnnnnnnnnnnnnnnnnnnnnnnnnnnnnnnnnnnnnnnnnnnnnnnnnnnnnnnnnnnnnnnnnnnnnnnnn | (SEQ ID NO:104) |
| nnnnnnnnnnnnnnnnnnnnnnnnnnnnnnnagn-3′, | |
| 5′-ncgnnnnnnnnnnnnnnnnnnnnnnnnnnnnnnnnnnnnnnnnnnnnnnnnnnnnnnnnnnnnnnnnnnnnnnnnnnnn | (SEQ ID NO:105) |
| nnnnnnnnnnnnnnnnnnnnnnnnnnnnnnnnagn-3′, | |
| 5′-ncgnnnnnnnnnnnnnnnnnnnnnnnnnnnnnnnnnnnnnnnnnnnnnnnnnnnnnnnnnnnnnnnnnnnnnnnnnnnn | (SEQ ID NO:106) |
| nnnnnnnnnnnnnnnnnnnnnnnnnnnnnnnnnagn-3′, | |
| 5′-ncgnnnnnnnnnnnnnnnnnnnnnnnnnnnnnnnnnnnnnnnnnnnnnnnnnnnnnnnnnnnnnnnnnnnnnnnnnnnn | (SEQ ID NO:107) |
| nnnnnnnnnnnnnnnnnnnnnnnnnnnnnnnnnnagn-3′, | |
| 5′-ncgnnnnnnnnnnnnnnnnnnnnnnnnnnnnnnnnnnnnnnnnnnnnnnnnnnnnnnnnnnnnnnnnnnnnnnnnnnnn | (SEQ ID NO:108) |
| nnnnnnnnnnnnnnnnnnnnnnnnnnnnnnnnnnnagn-3′, | |
| 5′-ncgnnnnnnnnnnnnnnnnnnnnnnnnnnnnnnnnnnnnnnnnnnnnnnnnnnnnnnnnnnnnnnnnnnnnnnnnnnnn | (SEQ ID NO:109) |
| nnnnnnnnnnnnnnnnnnnnnnnnnnnnnnnnnnnnagn-3′, | |
| 5′-ncgnnnnnnnnnnnnnnnnnnnnnnnnnnnnnnnnnnnnnnnnnnnnnnnnnnnnnnnnnnnnnnnnnnnnnnnnnnnn | (SEQ ID NO:110) |
| nnnnnnnnnnnnnnnnnnnnnnnnnnnnnnnnnnnnnagn-3′, | |
| 5′-ncgnnnnnnnnnnnnnnnnnnnnnnnnnnnnnnnnnnnnnnnnnnnnnnnnnnnnnnnnnnnnnnnnnnnnnnnnnnnn | (SEQ ID NO:111) |
| nnnnnnnnnnnnnnnnnnnnnnnnnnnnnnnnnnnnnnagn-3′, | |
| 5′-ncgnnnnnnnnnnnnnnnnnnnnnnnnnnnnnnnnnnnnnnnnnnnnnnnnnnnnnnnnnnnnnnnnnnnnnnnnnnnn | (SEQ ID NO:112) |
| nnnnnnnnnnnnnnnnnnnnnnnnnnnnnnnnnnnnnnnagn-3′, | |
| 5′-ncgnnnnnnnnnnnnnnnnnnnnnnnnnnnnnnnnnnnnnnnnnnnnnnnnnnnnnnnnnnnnnnnnnnnnnnnnnnnn | (SEQ ID NO:113) |
| nnnnnnnnnnnnnnnnnnnnnnnnnnnnnnnnnnnnnnnnagn-3′, | |
| 5′-ncgnnnnnnnnnnnnnnnnnnnnnnnnnnnnnnnnnnnnnnnnnnnnnnnnnnnnnnnnnnnnnnnnnnnnnnnnnnnn | (SEQ ID NO:114) |
| nnnnnnnnnnnnnnnnnnnnnnnnnnnnnnnnnnnnnnnnnagn-3′, | |
| 5′-ncgnnnnnnnnnnnnnnnnnnnnnnnnnnnnnnnnnnnnnnnnnnnnnnnnnnnnnnnnnnnnnnnnnnnnnnnnnnnn | (SEQ ID NO:115) |
| nnnnnnnnnnnnnnnnnnnnnnnnnnnnnnnnnnnnnnnnnnagn-3′, | |
| 5′-ncgnnnnnnnnnnnnnnnnnnnnnnnnnnnnnnnnnnnnnnnnnnnnnnnnnnnnnnnnnnnnnnnnnnnnnnnnnnnn | (SEQ ID NO:116) |
| nnnnnnnnnnnnnnnnnnnnnnnnnnnnnnnnnnnnnnnnnnnagn-3′, | |
| 5′-ncgnnnnnnnnnnnnnnnnnnnnnnnnnnnnnnnnnnnnnnnnnnnnnnnnnnnnnnnnnnnnnnnnnnnnnnnnnnnn | (SEQ ID NO:117) |
| nnnnnnnnnnnnnnnnnnnnnnnnnnnnnnnnnnnnnnnnnnnnagn-3′, | |
| 5′-ncgnnnnnnnnnnnnnnnnnnnnnnnnnnnnnnnnnnnnnnnnnnnnnnnnnnnnnnnnnnnnnnnnnnnnnnnnnnnn | (SEQ ID NO:118) |
| nnnnnnnnnnnnnnnnnnnnnnnnnnnnnnnnnnnnnnnnnnnnnagn-3′, | |
| 5′-ncgnnnnnnnnnnnnnnnnnnnnnnnnnnnnnnnnnnnnnnnnnnnnnnnnnnnnnnnnnnnnnnnnnnnnnnnnnnnn | (SEQ ID NO:119) |
| nnnnnnnnnnnnnnnnnnnnnnnnnnnnnnnnnnnnnnnnnnnnnnagn-3′, | |
| 5′-ncgnnnnnnnnnnnnnnnnnnnnnnnnnnnnnnnnnnnnnnnnnnnnnnnnnnnnnnnnnnnnnnnnnnnnnnnnnnnn | (SEQ ID NO:120) |
| nnnnnnnnnnnnnnnnnnnnnnnnnnnnnnnnnnnnnnnnnnnnnnnagn-3′, | |
| 5′-ncgnnnnnnnnnnnnnnnnnnnnnnnnnnnnnnnnnnnnnnnnnnnnnnnnnnnnnnnnnnnnnnnnnnnnnnnnnnnn | (SEQ ID NO:121) |
| nnnnnnnnnnnnnnnnnnnnnnnnnnnnnnnnnnnnnnnnnnnnnnnnagn-3′, | |
| 5′-ncgnnnnnnnnnnnnnnnnnnnnnnnnnnnnnnnnnnnnnnnnnnnnnnnnnnnnnnnnnnnnnnnnnnnnnnnnnnnn | (SEQ ID NO:122) |
| nnnnnnnnnnnnnnnnnnnnnnnnnnnnnnnnnnnnnnnnnnnnnnnnnagn-3′, | |
| 5′-ncgnnnnnnnnnnnnnnnnnnnnnnnnnnnnnnnnnnnnnnnnnnnnnnnnnnnnnnnnnnnnnnnnnnnnnnnnnnnn | (SEQ ID NO:123) |
| nnnnnnnnnnnnnnnnnnnnnnnnnnnnnnnnnnnnnnnnnnnnnnnnnnagn-3′, | |
| 5′-ncgnnnnnnnnnnnnnnnnnnnnnnnnnnnnnnnnnnnnnnnnnnnnnnnnnnnnnnnnnnnnnnnnnnnnnnnnnnnn | (SEQ ID NO:124) |
| nnnnnnnnnnnnnnnnnnnnnnnnnnnnnnnnnnnnnnnnnnnnnnnnnnnagn-3′, | |
| 5′-ncgnnnnnnnnnnnnnnnnnnnnnnnnnnnnnnnnnnnnnnnnnnnnnnnnnnnnnnnnnnnnnnnnnnnnnnnnnnnn | (SEQ ID NO:125) |
| nnnnnnnnnnnnnnnnnnnnnnnnnnnnnnnnnnnnnnnnnnnnnnnnnnnnagn-3′, | |
| 5′-ncgnnnnnnnnnnnnnnnnnnnnnnnnnnnnnnnnnnnnnnnnnnnnnnnnnnnnnnnnnnnnnnnnnnnnnnnnnnnn | (SEQ ID NO:126) |
| nnnnnnnnnnnnnnnnnnnnnnnnnnnnnnnnnnnnnnnnnnnnnnnnnnnnnagn-3′, | |
| 5′-ncgnnnnnnnnnnnnnnnnnnnnnnnnnnnnnnnnnnnnnnnnnnnnnnnnnnnnnnnnnnnnnnnnnnnnnnnnnnnn | (SEQ ID NO:127) |
| nnnnnnnnnnnnnnnnnnnnnnnnnnnnnnnnnnnnnnnnnnnnnnnnnnnnnnagn-3′, | |
| 5′-ncgnnnnnnnnnnnnnnnnnnnnnnnnnnnnnnnnnnnnnnnnnnnnnnnnnnnnnnnnnnnnnnnnnnnnnnnnnnnn | (SEQ ID NO:128) |
| nnnnnnnnnnnnnnnnnnnnnnnnnnnnnnnnnnnnnnnnnnnnnnnnnnnnnnnagn-3′, | |
| 5′-ncgnnnnnnnnnnnnnnnnnnnnnnnnnnnnnnnnnnnnnnnnnnnnnnnnnnnnnnnnnnnnnnnnnnnnnnnnnnnn | (SEQ ID NO:129) |
| nnnnnnnnnnnnnnnnnnnnnnnnnnnnnnnnnnnnnnnnnnnnnnnnnnnnnnnnagn-3′, | |
| 5′-ncgnnnnnnnnnnnnnnnnnnnnnnnnnnnnnnnnnnnnnnnnnnnnnnnnnnnnnnnnnnnnnnnnnnnnnnnnnnnn | (SEQ ID NO:130) |
| nnnnnnnnnnnnnnnnnnnnnnnnnnnnnnnnnnnnnnnnnnnnnnnnnnnnnnnnnagn-3′, | |
| 5′-ncgnnnnnnnnnnnnnnnnnnnnnnnnnnnnnnnnnnnnnnnnnnnnnnnnnnnnnnnnnnnnnnnnnnnnnnnnnnnn | (SEQ ID NO:131) |
| nnnnnnnnnnnnnnnnnnnnnnnnnnnnnnnnnnnnnnnnnnnnnnnnnnnnnnnnnnagn-3′, | |
| 5′-ncgnnnnnnnnnnnnnnnnnnnnnnnnnnnnnnnnnnnnnnnnnnnnnnnnnnnnnnnnnnnnnnnnnnnnnnnnnnnn | (SEQ ID NO:132) |
| nnnnnnnnnnnnnnnnnnnnnnnnnnnnnnnnnnnnnnnnnnnnnnnnnnnnnnnnnnnagn-3′, | |
| 5′-ncgnnnnnnnnnnnnnnnnnnnnnnnnnnnnnnnnnnnnnnnnnnnnnnnnnnnnnnnnnnnnnnnnnnnnnnnnnnnn | (SEQ ID NO:133) |
| nnnnnnnnnnnnnnnnnnnnnnnnnnnnnnnnnnnnnnnnnnnnnnnnnnnnnnnnnnnnagn-3′, | |
| 5′-ncgnnnnnnnnnnnnnnnnnnnnnnnnnnnnnnnnnnnnnnnnnnnnnnnnnnnnnnnnnnnnnnnnnnnnnnnnnnnn | (SEQ ID NO:134) |
| nnnnnnnnnnnnnnnnnnnnnnnnnnnnnnnnnnnnnnnnnnnnnnnnnnnnnnnnnnnnnagn-3′, | |
| 5′-ncgnnnnnnnnnnnnnnnnnnnnnnnnnnnnnnnnnnnnnnnnnnnnnnnnnnnnnnnnnnnnnnnnnnnnnnnnnnnn | (SEQ ID NO:135) |
| nnnnnnnnnnnnnnnnnnnnnnnnnnnnnnnnnnnnnnnnnnnnnnnnnnnnnnnnnnnnnnagn-3′, | |
| 5′-ncgnnnnnnnnnnnnnnnnnnnnnnnnnnnnnnnnnnnnnnnnnnnnnnnnnnnnnnnnnnnnnnnnnnnnnnnnnnnn | (SEQ ID NO:136) |
| nnnnnnnnnnnnnnnnnnnnnnnnnnnnnnnnnnnnnnnnnnnnnnnnnnnnnnnnnnnnnnnagn-3′, | |
| 5′-ncgnnnnnnnnnnnnnnnnnnnnnnnnnnnnnnnnnnnnnnnnnnnnnnnnnnnnnnnnnnnnnnnnnnnnnnnnnnnn | (SEQ ID NO:137) |
| nnnnnnnnnnnnnnnnnnnnnnnnnnnnnnnnnnnnnnnnnnnnnnnnnnnnnnnnnnnnnnnnagn-3′, | |
| 5′-ncgnnnnnnnnnnnnnnnnnnnnnnnnnnnnnnnnnnnnnnnnnnnnnnnnnnnnnnnnnnnnnnnnnnnnnnnnnnnn | (SEQ ID NO:138) |
| nnnnnnnnnnnnnnnnnnnnnnnnnnnnnnnnnnnnnnnnnnnnnnnnnnnnnnnnnnnnnnnnnagn-3′, | |
| 5′-ncgnnnnnnnnnnnnnnnnnnnnnnnnnnnnnnnnnnnnnnnnnnnnnnnnnnnnnnnnnnnnnnnnnnnnnnnnnnnn | (SEQ ID NO:139) |
| nnnnnnnnnnnnnnnnnnnnnnnnnnnnnnnnnnnnnnnnnnnnnnnnnnnnnnnnnnnnnnnnnnagn-3′, | |
| 5′-ncgnnnnnnnnnnnnnnnnnnnnnnnnnnnnnnnnnnnnnnnnnnnnnnnnnnnnnnnnnnnnnnnnnnnnnnnnnnnn | (SEQ ID NO:140) |
| nnnnnnnnnnnnnnnnnnnnnnnnnnnnnnnnnnnnnnnnnnnnnnnnnnnnnnnnnnnnnnnnnnnagn-3′, | |
| 5′-ncgnnnnnnnnnnnnnnnnnnnnnnnnnnnnnnnnnnnnnnnnnnnnnnnnnnnnnnnnnnnnnnnnnnnnnnnnnnnn | (SEQ ID NO:141) |
| nnnnnnnnnnnnnnnnnnnnnnnnnnnnnnnnnnnnnnnnnnnnnnnnnnnnnnnnnnnnnnnnnnnnagn-3′, | |
| 5′-ncgnnnnnnnnnnnnnnnnnnnnnnnnnnnnnnnnnnnnnnnnnnnnnnnnnnnnnnnnnnnnnnnnnnnnnnnnnnnn | (SEQ ID NO:142) |
| nnnnnnnnnnnnnnnnnnnnnnnnnnnnnnnnnnnnnnnnnnnnnnnnnnnnnnnnnnnnnnnnnnnnnagn-3′, | |
| 5′-ncgnnnnnnnnnnnnnnnnnnnnnnnnnnnnnnnnnnnnnnnnnnnnnnnnnnnnnnnnnnnnnnnnnnnnnnnnnnnn | (SEQ ID NO:143) |
| nnnnnnnnnnnnnnnnnnnnnnnnnnnnnnnnnnnnnnnnnnnnnnnnnnnnnnnnnnnnnnnnnnnnnnagn-3′, | |
| 5′-ncgnnnnnnnnnnnnnnnnnnnnnnnnnnnnnnnnnnnnnnnnnnnnnnnnnnnnnnnnnnnnnnnnnnnnnnnnnnnn | (SEQ ID NO:144) |
| nnnnnnnnnnnnnnnnnnnnnnnnnnnnnnnnnnnnnnnnnnnnnnnnnnnnnnnnnnnnnnnnnnnnnnnagn-3′, | |
| 5′-ncgnnnnnnnnnnnnnnnnnnnnnnnnnnnnnnnnnnnnnnnnnnnnnnnnnnnnnnnnnnnnnnnnnnnnnnnnnnnn | (SEQ ID NO:145) |
| nnnnnnnnnnnnnnnnnnnnnnnnnnnnnnnnnnnnnnnnnnnnnnnnnnnnnnnnnnnnnnnnnnnnnnnnagn-3′, | |
| 5′-ncgnnnnnnnnnnnnnnnnnnnnnnnnnnnnnnnnnnnnnnnnnnnnnnnnnnnnnnnnnnnnnnnnnnnnnnnnnnnn | (SEQ ID NO:146) |
| nnnnnnnnnnnnnnnnnnnnnnnnnnnnnnnnnnnnnnnnnnnnnnnnnnnnnnnnnnnnnnnnnnnnnnnnnagn-3′, | |
| 5′-ncgnnnnnnnnnnnnnnnnnnnnnnnnnnnnnnnnnnnnnnnnnnnnnnnnnnnnnnnnnnnnnnnnnnnnnnnnnnnn | (SEQ ID NO:147) |
| nnnnnnnnnnnnnnnnnnnnnnnnnnnnnnnnnnnnnnnnnnnnnnnnnnnnnnnnnnnnnnnnnnnnnnnnnnagn-3′, | |
| 5′-ncgnnnnnnnnnnnnnnnnnnnnnnnnnnnnnnnnnnnnnnnnnnnnnnnnnnnnnnnnnnnnnnnnnnnnnnnnnnnn | (SEQ ID NO:148) |
| nnnnnnnnnnnnnnnnnnnnnnnnnnnnnnnnnnnnnnnnnnnnnnnnnnnnnnnnnnnnnnnnnnnnnnnnnnnagn-3′, | |
| 5′-ncgnnnnnnnnnnnnnnnnnnnnnnnnnnnnnnnnnnnnnnnnnnnnnnnnnnnnnnnnnnnnnnnnnnnnnnnnnnnn | (SEQ ID NO:149) |
| nnnnnnnnnnnnnnnnnnnnnnnnnnnnnnnnnnnnnnnnnnnnnnnnnnnnnnnnnnnnnnnnnnnnnnnnnnnnagn-3′, | |
| 5′-ncgnnnnnnnnnnnnnnnnnnnnnnnnnnnnnnnnnnnnnnnnnnnnnnnnnnnnnnnnnnnnnnnnnnnnnnnnnnnn | (SEQ ID NO:150) |
| nnnnnnnnnnnnnnnnnnnnnnnnnnnnnnnnnnnnnnnnnnnnnnnnnnnnnnnnnnnnnnnnnnnnnnnnnnnnnagn-3′, | |
| 5′-ncgnnnnnnnnnnnnnnnnnnnnnnnnnnnnnnnnnnnnnnnnnnnnnnnnnnnnnnnnnnnnnnnnnnnnnnnnnnnn | (SEQ ID NO:151) |
| nnnnnnnnnnnnnnnnnnnnnnnnnnnnnnnnnnnnnnnnnnnnnnnnnnnnnnnnnnnnnnnnnnnnnnnnnnnnnnagn-3′, | |
| 5′-ncgnnnnnnnnnnnnnnnnnnnnnnnnnnnnnnnnnnnnnnnnnnnnnnnnnnnnnnnnnnnnnnnnnnnnnnnnnnnn | (SEQ ID NO:152) |
| nnnnnnnnnnnnnnnnnnnnnnnnnnnnnnnnnnnnnnnnnnnnnnnnnnnnnnnnnnnnnnnnnnnnnnnnnnnnnnnagn-3′, | |
| 5′-ncgnnnnnnnnnnnnnnnnnnnnnnnnnnnnnnnnnnnnnnnnnnnnnnnnnnnnnnnnnnnnnnnnnnnnnnnnnnnn | (SEQ ID NO:153) |
| nnnnnnnnnnnnnnnnnnnnnnnnnnnnnnnnnnnnnnnnnnnnnnnnnnnnnnnnnnnnnnnnnnnnnnnnnnnnnnnnag | |
| n-3′, | |
| or | |
| 5′-ncgnnnnnnnnnnnnnnnnnnnnnnnnnnnnnnnnnnnnnnnnnnnnnnnnnnnnnnnnnnnnnnnnnnnnnnnnnnnn | (SEQ ID NO:154) |
| nnnnnnnnnnnnnnnnnnnnnnnnnnnnnnnnnnnnnnnnnnnnnnnnnnnnnnnnnnnnnnnnnnnnnnnnnnnnnnnnna | |
| gn-3′ | |

(bolded nucleotides indicate preferred basepairing;
n is any nucleotide).

(bolded nucleotides indicate preferred basepairing; n is any nucleotide). Consensus site 5 is depicted in FIG. 1. Consensus site 6 comprises a region of RNA comprising a first and second polynucleotide. The first polynucleotide comprises from about four nucleotides to about eleven nucleotides, wherein portions of the polynucleotide form a double-stranded RNA having the following features (5′ to 3′): a first side of a stem comprising from about three nucleotides to about nine nucleotides wherein a bulge comprising from about one nucleotide to about two nucleotides is present in the first side of the stem. The second polynucleotide comprises from about three nucleotides to about nine nucleotides, wherein portions of the polynucleotide form a double-stranded RNA having the following features (5′ to 3′): a second side of the stem comprising from about three nucleotides to about nine nucleotides.

In regard to consensus site 6, the first polynucleotide preferably comprises seven nucleotides, wherein portions of the polynucleotide form a double-stranded RNA having the following features (5′ to 3′): a first side of a stem comprising six nucleotides wherein a bulge comprising one nucleotide is present between the fourth and fifth nucleotides of the first side of the stem. Preferably, the first polynucleotide comprises the sequence 5′-nnnnann-3′ (bolded nucleotides indicate preferred basepairing; n is any nucleotide). The second polynucleotide preferably comprises six nucleotides, wherein portions of the polynucleotide form a double-stranded RNA having the following features (5′ to 3′): a second side of the stem comprising six nucleotides. Preferably, the second polynucleotide comprises the sequence 5′-nnnnnn-3′ (bolded nucleotides indicate preferred basepairing; n is any nucleotide). Consensus site 6 is depicted in FIG. 1.

Consensus site 7 comprises a region of RNA comprising a first and second polynucleotide. The first polynucleotide comprises from about six nucleotides to about fourteen nucleotides, wherein portions of the polynucleotide form a double-stranded RNA having the following features (5′ to 3′): a dangling region comprising from about one nucleotide to about two nucleotides, a first side of a stem comprising from about three nucleotides to about seven nucleotides wherein a first side of an internal loop comprising from about two nucleotides to about five nucleotides is present in the first side of the stem. The second polynucleotide comprises from about four nucleotides to about ten nucleotides, wherein portions of the polynucleotide form a double-stranded RNA having the following features (5′ to 3′): a second side of the stem comprising from about three nucleotides to about seven nucleotides wherein a second side of the internal loop comprising from about one nucleotide to about three nucleotides is present in the second side of the stem.

In regard to consensus site 7, the first polynucleotide preferably comprises nine nucleotides, wherein portions of the polynucleotide form a double-stranded RNA having the following features (5′ to 3′): a dangling region comprising one nucleotide, a first side of a stem comprising five nucleotides wherein a first side of an internal loop comprising three nucleotides is present between the second and third nucleotides of the first side of the stem. Preferably, the first polynucleotide comprises the sequence 5′-annunccnn-3′ (bolded nucleotides indicate preferred basepairing; n is any nucleotide). The second polynucleotide preferably comprises seven nucleotides, wherein portions of the polynucleotide form a double-stranded RNA having the following features (5′ to 3′): a second side of the stem comprising five nucleotides wherein a second side of the internal loop comprising two nucleotides is present between the third and fourth nucleotides of the second side of the stem. Preferably, the second polynucleotide comprises the sequence 5′-nngannn-3′ (bolded nucleotides indicate preferred basepairing; n is any nucleotide). Consensus site 7 is depicted in FIG. 1.

Consensus site 8 comprises a region of RNA comprising a first and second polynucleotide. The first polynucleotide comprises from about five nucleotides to about fourteen nucleotides, wherein portions of the polynucleotide form a double-stranded RNA having the following features (5′ to 3′): a first side of a stem comprising from about three nucleotides to about nine nucleotides wherein a first side of an internal loop comprising from about two nucleotides to about five nucleotides is present in the first side of the stem. The second polynucleotide comprises from about five nucleotides to about fifteen nucleotides, wherein portions of the polynucleotide form a double-stranded RNA having the following features (5′ to 3′): a second side of the stem comprising from about three nucleotides to about nine nucleotides wherein a second side of the internal loop comprising from about two nucleotides to about six nucleotides is present in the second side of the stem.

In regard to consensus site 8, the first polynucleotide preferably comprises nine nucleotides, wherein portions of the polynucleotide form a double-stranded RNA having the following features (5′ to 3′): a first side of a stem comprising six nucleotides wherein a first side of an internal loop comprising three nucleotides is present between the third and fourth nucleotides of the first side of the stem. Preferably, the first polynucleotide comprises the sequence 5′-ggnanannn-3′ (bolded nucleotides indicate preferred basepairing; n is any nucleotide). The second polynucleotide preferably comprises ten nucleotides, wherein portions of the polynucleotide form a double-stranded RNA having the following features (5′ to 3′): a second side of the stem comprising six nucleotides wherein a second side of the internal loop comprising four nucleotides is present between the third and fourth nucleotides of the second side of the stem. Preferably, the second polynucleotide comprises the sequence 5′-nnnuaauacc-3′ (SEQ ID NO:155) (bolded nucleotides indicate preferred basepairing; n is any nucleotide). Consensus site 8 is depicted in FIG. 1.

Consensus site 9 comprises a region of RNA comprising a polynucleotide comprising from about six nucleotides to about sixteen nucleotides, wherein portions of the polynucleotide form a double-stranded RNA having the following features (5′ to 3′): a first side of a stem comprising from about two nucleotides to about five nucleotides, a terminal loop comprising from about two nucleotides to about six nucleotides, and a second side of the stem comprising from about two nucleotides to about five nucleotides.

In regard to consensus site 9, the polynucleotide preferably comprises ten nucleotides, wherein portions of the polynucleotide form a double-stranded RNA having the following features (5′ to 3′): a first side of a stem comprising three nucleotides, a terminal loop comprising four nucleotides, and a second side of the stem comprising three nucleotides. Preferably, the polynucleotide comprises the sequence 5′-nnngaaannn-3′ (SEQ ID NO:156) (bolded nucleotides indicate preferred basepairing; n is any nucleotide). Consensus site 9 is depicted in FIG. 1.

Consensus site 10 comprises a region of RNA comprising a polynucleotide comprising from about six nucleotides to about sixteen nucleotides, wherein portions of the polynucleotide form a double-stranded RNA having the following features (5′ to 3′): a first side of a stem comprising from about two nucleotides to about five nucleotides, a terminal loop comprising from about two nucleotides to about six nucleotides, and a second side of the stem comprising from about two nucleotides to about five nucleotides.

In regard to consensus site 10, the polynucleotide preferably comprises ten nucleotides, wherein portions of the polynucleotide form a double-stranded RNA having the following features (5′ to 3′): a first side of a stem comprising three nucleotides, a terminal loop comprising four nucleotides, and a second side of the stem comprising three nucleotides. Preferably, the polynucleotide comprises the sequence 5′-nnnnnnnnnn-3′ (SEQ ID NO:157) (bolded nucleotides indicate preferred basepairing; n is any nucleotide). Consensus site 10 is depicted in FIG. 1.

Consensus site 11 comprises a region of RNA comprising a first and second polynucleotide. The first polynucleotide comprises from about seven nucleotides to about seven nucleotides, wherein portions of the polynucleotide form a double-stranded RNA having the following features (5′ to 3′): a first side of a stem comprising from about four nucleotides to about ten nucleotides wherein a first side of an internal loop comprising from about three nucleotides to about seven nucleotides is present in the first side of the stem. The second polynucleotide comprises from about six nucleotides to about fifteen nucleotides, wherein portions of the polynucleotide form a double-stranded RNA having the following features (5′ to 3′): a second side of the stem comprising from about four nucleotides to about ten nucleotides wherein a bulge comprising from about one nucleotide to about three nucleotides is present in the second side of the stem and wherein a second side of the internal loop comprising from about one nucleotide to about two nucleotides is present in the second side of the stem.

In regard to consensus site 11, the first polynucleotide preferably comprises twelve nucleotides, wherein portions of the polynucleotide form a double-stranded RNA having the following features (5′ to 3′): a first side of a stem comprising seven nucleotides wherein a first side of an internal loop comprising five nucleotides is present between the third and fourth nucleotides of the first side of the stem. Preferably, the first polynucleotide comprises the sequence 5′-gncnnngannnn-3′ (SEQ ID NO:158) (bolded nucleotides indicate preferred basepairing; n is any nucleotide). The second. polynucleotide preferably comprises ten nucleotides, wherein portions of the polynucleotide form a double-stranded RNA having the following features (5′ to 3′): a second side of the stem comprising seven nucleotides wherein a bulge comprising two nucleotides is present between the third and fourth nucleotides of the second side of the stem and wherein a second side of the internal loop comprising one nucleotide is present between the fourth and fifth nucleotides of the second side of the stem. Preferably, the second polynucleotide comprises the sequence 5′-nnnaunagnu-3′ (SEQ ID NO:159) (bolded nucleotides indicate preferred basepairing; n is any nucleotide). Consensus site 11 is depicted in FIG. 1.

Consensus site 12 comprises a region of RNA comprising a polynucleotide comprising from about eighteen nucleotides to about forty eight nucleotides, wherein portions of the polynucleotide form a double-stranded RNA having the following features (5′ to 3′): a first side of a stem comprising from about six nucleotides to about sixteen nucleotides wherein a bulge comprising from about one nucleotide to about three nucleotides is present in the first side of the stem, a terminal loop comprising from about four nucleotides to about ten nucleotides, a second side of the stem comprising from about six nucleotides to about sixteen nucleotides, and dangling region comprising from about one nucleotide to about three nucleotides.

In regard to consensus site 12, the polynucleotide preferably comprises thirty three nucleotides, wherein portions of the polynucleotide form a double-stranded RNA having the following features (5′ to 3′): a first side of a stem comprising eleven nucleotides wherein a bulge comprising two nucleotides is present between the third and fourth nucleotides of the first side of the stem, a terminal loop comprising seven nucleotides, a second side of the stem comprising eleven nucleotides, and dangling region comprising two nucleotides. Preferably, the polynucleotide comprises the sequence 5′-gnunguuggunngguaanggcnnaccaagncnn-3′ (SEQ ID NO:160) (bolded nucleotides indicate preferred basepairing; n is any nucleotide). Consensus site 12 is depicted in FIG. 1. The molecular interaction site comprises a drug-binding pocket encompassing an area defined by 14 Å by 15 Å and is located in the major groove side of stem 11 and faces the Hoogsteen side of G251.

Consensus site 13 comprises a region of RNA comprising a polynucleotide comprising from about twelve nucleotides to about thirty one nucleotides, wherein portions of the polynucleotide form a double-stranded RNA having the following features (5′ to 3′): a first side of a stem comprising from about four nucleotides to about ten nucleotides, a terminal loop comprising from about two nucleotides to about six nucleotides, and a second side of the stem comprising from about four nucleotides to about ten nucleotides wherein a bulge comprising from about two nucleotides to about five nucleotides is present in the second side of the stem.

In regard to consensus site 13, the polynucleotide preferably comprises twenty one nucleotides, wherein portions of the polynucleotide form a double-stranded RNA having the following features (5′ to 3′): a first side of a stem comprising seven nucleotides, a terminal loop comprising four nucleotides, and a second side of the stem comprising seven nucleotides wherein a bulge comprising three nucleotides is present between the fourth and fifth nucleotides of the second side of the stem. Preferably, the polynucleotide comprises the sequence 5′-cngnncugagaggnngnncng-3′ (SEQ ID NO:161) (bolded nucleotides indicate preferred basepairing; n is any nucleotide). Consensus site 13 is depicted in FIG. 1.

Consensus site 14 comprises a region of RNA comprising a polynucleotide comprising from about eleven nucleotides to about twenty nine nucleotides, wherein portions of the polynucleotide form a double-stranded RNA having the following features (5′ to 3′): a first side of a stem comprising from about three nucleotides to about seven nucleotides, a terminal loop comprising from about five nucleotides to about fifteen nucleotides, and a second side of the stem comprising from about three nucleotides to about seven nucleotides.

In regard to consensus site 14, the polynucleotide preferably comprises twenty nucleotides, wherein portions of the polynucleotide form a double-stranded RNA having the following features (5′ to 3′): a first side of a stem comprising five nucleotides, a terminal loop comprising ten nucleotides, and a second side of the stem comprising five nucleotides. Preferably, the polynucleotide comprises the sequence 5′-uggnacugaganacggncca-3′ (SEQ ID NO:162) (bolded nucleotides indicate preferred basepairing; n is any nucleotide). Consensus site 14 is depicted in FIG. 1.

Consensus site 15 comprises a region of RNA comprising a polynucleotide comprising from about six nucleotides to about sixteen nucleotides, wherein portions of the polynucleotide form a double-stranded RNA having the following features (5′ to 3′): a first side of a stem comprising from about two nucleotides to about five nucleotides, a terminal loop comprising from about two nucleotides to about six nucleotides, and a second side of the stem comprising from about two nucleotides to about five nucleotides.

In regard to consensus site 15, the polynucleotide preferably comprises ten nucleotides, wherein portions of the polynucleotide form a double-stranded RNA having the following features (5′ to 3′): a first side of a stem comprising three nucleotides, a terminal loop comprising four nucleotides, and a second side of the stem comprising three nucleotides. Preferably, the polynucleotide comprises the sequence 5′-uccuacggga-3′ (SEQ ID NO:163) (bolded nucleotides indicate preferred basepairing; n is any nucleotide). Consensus site 15 is depicted in FIG. 1.

Consensus site 16 comprises a region of RNA comprising a polynucleotide comprising from about thirteen nucleotides to about thirty seven nucleotides, wherein portions of the polynucleotide form a double-stranded RNA having the following features (5′ to 3′): a first side of a stem comprising from about four nucleotides to about twelve nucleotides wherein a first side of an internal loop comprising from about two nucleotides to about five nucleotides is present in the first side of the stem, a terminal loop comprising from about two nucleotides to about six nucleotides, and a second side of the stem comprising from about four nucleotides to about twelve nucleotides wherein a second side of the internal loop comprising from about one nucleotide to about two nucleotides is present in the second side of the stem.

In regard to consensus site 16, the polynucleotide preferably comprises twenty four nucleotides, wherein portions of the polynucleotide form a double-stranded RNA having the following features (5′ to 3′): a first side of a stem comprising eight nucleotides wherein a first side of an internal loop comprising three nucleotides is present between the third and fourth nucleotides of the first side of the stem, a terminal loop comprising four nucleotides, and a second side of the stem comprising eight nucleotides wherein a second side of the internal loop comprising one nucleotide is present between the fifth and sixth nucleotides of the second side of the stem. Preferably, the polynucleotide comprises the sequence 5′-nnncaauggnngnaanncugannn-3′ (SEQ ID NO:164) (bolded nucleotides indicate preferred basepairing; n is any nucleotide). Consensus site 16 is depicted in FIG. 1.

Consensus site 17 comprises a region of RNA comprising a polynucleotide comprising from about sixteen nucleotides to about forty seven nucleotides, wherein portions of the polynucleotide form a double-stranded RNA having the following features (5′ to 3′): a first side of a stem comprising from about four nucleotides to about twelve nucleotides wherein a first side of an internal loop comprising from about three nucleotides to about nine nucleotides is present in the first side of the stem, a terminal loop comprising from about two nucleotides to about seven nucleotides, and a second side of the stem comprising from about four nucleotides to about twelve nucleotides wherein a second side of the internal loop comprising from about three nucleotides to about seven nucleotides is present in the second side of the stem.

In regard to consensus site 17, the polynucleotide preferably comprises thirty to thirty two nucleotides, wherein portions of the polynucleotide form a double-stranded RNA having the following features (5′ to 3′): a first side of a stem comprising eight nucleotides wherein a first side of an internal loop comprising six nucleotides is present between the third and fourth nucleotides of the first side of the stem, a terminal loop comprising from three to five nucleotides, and a second side of the stem comprising eight nucleotides wherein a second side of the internal loop comprising five nucleotides is present between the fifth and sixth nucleotides of the second side of the stem. Preferably, the polynucleotide comprises the sequence 5′-gnnnganganggnnunngnunguaaannn-3′ (SEQ ID NO:165), 5 ′-gnnnganganggnnunnngnunguaaannn-3′ (SEQ ID NO:166), or 5′-gnnnganganggnnunnnngnunguaaannn-3′ (SEQ ID NO:167) (bolded nucleotides indicate preferred basepairing; n is any nucleotide). Consensus site 17 is depicted in FIG. 1.

Consensus site 18 comprises a region of RNA comprising a first and second polynucleotide. The first polynucleotide comprises from about five nucleotides to about thirteen nucleotides, wherein portions of the polynucleotide form a double-stranded -RNA having the following features (5′ to 3′): a first side of a stem comprising from about two nucleotides to about six nucleotides wherein a first side of an internal loop comprising from about two nucleotides to about five nucleotides is present in the first side of the stem, and a dangling region comprising from about one nucleotide to about two nucleotides. The second polynucleotide comprises from about five nucleotides to about fourteen nucleotides, wherein portions of the polynucleotide form a double-stranded RNA having the following features (5′ to 3′): a dangling region comprising from about one nucleotide to about two nucleotides, a second side of the stem comprising from about two nucleotides to about six nucleotides wherein a second side of the internal loop comprising from about two nucleotides to about six nucleotides is present in the second side of the stem.

In regard to consensus site 18, the first polynucleotide preferably comprises eight nucleotides, wherein portions of the polynucleotide form a double-stranded RNA having the following features (5′ to 3′): a first side of a stem comprising four nucleotides wherein a first side of an internal loop comprising three nucleotides is present between the third and fourth nucleotides of the first side of the stem, and a dangling region comprising one nucleotide. Preferably, the first polynucleotide comprises the sequence 5′-nnnganga-3′ (bolded nucleotides indicate preferred basepairing; n is any nucleotide). The second polynucleotide preferably comprises nine nucleotides, wherein portions of the polynucleotide form a double-stranded RNA having the following features (5′ to 3′): a dangling region comprising one nucleotide, a second side of the stem comprising four nucleotides wherein a second side of the internal loop comprising four nucleotides is present between the first and second nucleotides of the second side of the stem. Preferably, the second polynucleotide comprises the sequence 5′-acnnuannn-3′ (bolded nucleotides indicate preferred basepairing; n is any nucleotide). Consensus site 18 is depicted in FIG. 1.

Consensus site 19 comprises a region of RNA comprising a polynucleotide comprising from about twenty two nucleotides to about sixty one nucleotides, wherein portions of the polynucleotide form a double-stranded RNA having the following features (5′ to 3′): a first side of a first stem comprising from about five nucleotides to about fifteen nucleotides wherein a bulge comprising from about three nucleotides to about nine nucleotides is present in the first side of the stem and wherein a first side of an internal loop comprising from about three nucleotides to about seven nucleotides is present in the first side of the stem, a terminal loop comprising from about two nucleotides to about six nucleotides, and a second side of the stem comprising from about five nucleotides to about fifteen nucleotides wherein a second side of the internal loop comprising from about four nucleotides to about ten nucleotides is present in the second side of the stem.

In regard to consensus site 19, the polynucleotide preferably comprises forty two nucleotides, wherein portions of the polynucleotide form a double-stranded RNA having the following features (5′ to 3′): a first side of a first stem comprising ten nucleotides wherein a bulge comprising six nucleotides is present between the third and fourth nucleotides of the first side of the stem and wherein a first side of an internal loop comprising five nucleotides is present between the eighth and ninth nucleotides of the first side of the stem, a terminal loop comprising four nucleotides, and a second side of the stem comprising ten nucleotides wherein a second side of the internal loop comprising seven nucleotides is present between the second and third nucleotides of the second side of the stem. Preferably, the polynucleotide comprises the sequence 5′-nncggcnaacuncgugccagcagccgcgguaauacgnaggnn-3′ (SEQ ID NO:168) (bolded nucleotides indicate preferred basepairing; n is any nucleotide). Consensus site 19 is depicted in FIG. 1.

Consensus site 20 comprises a region of RNA comprising a first and second polynucleotide. The first polynucleotide comprises from about six nucleotides to about fourteen nucleotides, wherein portions of the polynucleotide form a double-stranded RNA having the following features (5′ to 3′): a first side of a stem comprising from about four nucleotides to about ten nucleotides wherein a first side of an internal loop comprising from about one nucleotide to about two nucleotides is present in the first side of the stem and wherein a bulge comprising from about one nucleotide to about two nucleotides is present in the first side of the stem. The second polynucleotide comprises from about five nucleotides to about thirteen nucleotides, wherein portions of the polynucleotide form a double-stranded RNA having the following features (5′ to 3′): a second side of the stem comprising from about four nucleotides to about ten nucleotides wherein a second side of the internal loop comprising from about one nucleotide to about three nucleotides is present in the second side of the stem.

In regard to consensus site 20, the first polynucleotide preferably comprises nine nucleotides, wherein portions of the polynucleotide form a double-stranded RNA having the following features (5′ to 3′): a first side of a stem comprising seven nucleotides wherein a first side of an internal loop comprising one nucleotide is present between the third and fourth nucleotides of the first side of the stem and wherein a bulge comprising one nucleotide is present between the fourth and fifth nucleotides of the first side of the stem. Preferably, the first polynucleotide comprises the sequence 5′-nnngnaggn-3′ (bolded nucleotides indicate preferred basepairing; n is any nucleotide). The second polynucleotide preferably comprises nine nucleotides, wherein portions of the polynucleotide form a double-stranded RNA having the following features (5′ to 3′): a second side of the stem comprising seven nucleotides wherein a second side of the internal loop comprising two nucleotides is present between the fourth and fifth nucleotides of the second side of the stem. Preferably, the second polynucleotide comprises the sequence 5′-ncunannnn-3′ (bolded nucleotides indicate preferred basepairing; n is any nucleotide). Consensus site 20 is depicted in FIG. 2.

Consensus site 21 comprises a region of RNA comprising a first, second and third polynucleotide. The first polynucleotide comprises from about four nucleotides to about eleven nucleotides, wherein portions of the polynucleotide form a double-stranded RNA having the following features (5′ to 3′): a first side of a first stem comprising from about three nucleotides to about nine nucleotides wherein a bulge comprising from about one nucleotide to about two nucleotides is present in the first side of the first stem. The second polynucleotide comprises from about six nucleotides to about fifteen nucleotides, wherein portions of the polynucleotide form: a double-stranded RNA having the following features (5′ to 3′): a first side of a second stem comprising from about two nucleotides to about five nucleotides, a bulge comprising from about two nucleotides to about five nucleotides, and a second side of the first stem comprising from about two nucleotides to about five nucleotides that are basepaired to the first three nucleotides of the first side of the first stem. The third polynucleotide comprises from about six nucleotides to about fifteen nucleotides, wherein portions of the polynucleotide form a double-stranded RNA having the following features (5′ to 3′): a second side of the first stem comprising from about two nucleotides to about five nucleotides that are basepaired to the last three nucleotides of the first side of the first stem, a bulge comprising from about two nucleotides to about five nucleotides, and a second side of the second stem comprising from about two nucleotides to about five nucleotides.

In regard to consensus site 21, the first polynucleotide preferably comprises seven nucleotides, wherein portions of the polynucleotide form a double-stranded RNA having the following features (5′ to 3′): a first side of a first stem comprising six nucleotides wherein a bulge comprising one nucleotide is present between the third and fourth nucleotides of the first side of the first stem. Preferably, the first polynucleotide comprises the sequence 5′-ggnggnn-3′ (bolded nucleotides indicate preferred basepairing; n is any nucleotide). The second polynucleotide preferably comprises nine nucleotides, wherein portions of the polynucleotide form a double-stranded RNA having the following features (5′ to 3′): a first side of a second stem comprising three nucleotides, a bulge comprising three nucleotides, and a second side of the first stem comprising three nucleotides that are basepaired to the first three nucleotides of the first side of the first stem. Preferably, the second polynucleotide comprises the sequence 5′-acugacncu-3′ (bolded nucleotides indicate preferred basepairing; n is any nucleotide). The third polynucleotide preferably comprises nine nucleotides, wherein portions of the polynucleotide form a double-stranded RNA having the following features (5′ to 3′): a second side of the first stem comprising three nucleotides that are basepaired to the last three nucleotides of the first side of the first stem, a bulge comprising three nucleotides, and a second side of the second stem comprising three nucleotides. Preferably, the third polynucleotide comprises the sequence 5′-nncungagn-3′ (bolded nucleotides indicate preferred basepairing; n is any nucleotide). Consensus site 21 is depicted in FIG. 2.

Consensus site 22 comprises a region of RNA comprising a first and second polynucleotide. The first polynucleotide comprises from about five nucleotides to about fourteen nucleotides, wherein portions of the polynucleotide form a double-stranded RNA having the following features (5′ to 3′): a first side of a stem comprising from about four nucleotides to about twelve nucleotides wherein a bulge comprising from about one nucleotide to about two nucleotides is present in the first side of the stem. The second polynucleotide comprises from about five nucleotides to about fifteen nucleotides, wherein portions of the polynucleotide form a double-stranded RNA having the following features (5′ to 3′): a second side of the stem comprising from about four nucleotides to about twelve nucleotides wherein a bulge comprising from about one nucleotide to about three nucleotides is present in the second side of the stem.

In regard to consensus site 22, the first polynucleotide preferably comprises nine nucleotides, wherein portions of the polynucleotide form a double-stranded RNA having the following features (5′ to 3′): a first side of a stem comprising eight nucleotides wherein a bulge comprising one nucleotide is present between the third and fourth nucleotides of the first side of the stem. Preferably, the first polynucleotide comprises the sequence 5′-nnnnngunn-3′ (bolded nucleotides indicate preferred basepairing; n is any nucleotide). The second polynucleotide preferably comprises ten nucleotides, wherein portions of the polynucleotide form a double-stranded RNA having the following features (5′ to 3′): a second side of the stem comprising eight nucleotides wherein a bulge comprising two nucleotides is present between the third and fourth nucleotides of the second side of the stem. Preferably, the second polynucleotide comprises the sequence 5′-nnnnacnnnn-3′ (SEQ ID NO:169) (bolded nucleotides indicate preferred basepairing; n is any nucleotide). Consensus site 22 is depicted in FIG. 2.

Consensus site 23 comprises a region of RNA comprising a first and second polynucleotide. The first polynucleotide comprises from about six nucleotides to about sixteen nucleotides, wherein portions of the polynucleotide form a double-stranded RNA having the following features (5′ to 3′): a first side of a stem comprising from about four nucleotides to about ten nucleotides wherein a first side of an internal loop comprising from about two nucleotides to about six nucleotides is present in the first side of the stem. The second polynucleotide comprises from about five nucleotides to about thirteen nucleotides, wherein portions of the polynucleotide form a double-stranded RNA having the following features (5′ to 3′): a second side of the stem comprising from about four nucleotides to about ten nucleotides wherein a second side of the internal loop comprising from about one nucleotide to about three nucleotides is present in the second side of the stem.

In regard to consensus site 23, the first polynucleotide preferably comprises eleven nucleotides, wherein portions of the polynucleotide form a double-stranded RNA having the following features (5′ to 3′): a first side of a stem comprising seven nucleotides wherein a first side of an internal loop comprising four nucleotides is present between the third and fourth nucleotides of the first side of the stem. Preferably, the first polynucleotide comprises the sequence 5′-gunaaannnnn-3′ (SEQ ID NO:170) (bolded nucleotides indicate preferred basepairing; n is any nucleotide). The second polynucleotide preferably comprises eight or nine nucleotides, wherein portions of the polynucleotide form a double-stranded RNA having the following features (5′ to 3′): a second side of the stem comprising seven nucleotides wherein a second side of the internal loop comprising one or two nucleotides is present between the fourth and fifth nucleotides of the second side of the stem. Preferably, the second polynucleotide comprises the sequence 5′-nnnnnnnc-3′ or 5′-nnnnnnnnc-3′ (bolded nucleotides indicate preferred basepairing; n is any nucleotide). Consensus site 23 is depicted in FIG. 2.

Consensus site 24 comprises a region of RNA comprising a first and second polynucleotide. The first polynucleotide comprises from about nine nucleotides to about twenty four nucleotides, wherein portions of the polynucleotide form a double-stranded RNA having the following features (5′ to 3′): a first side of a stem comprising from about seven nucleotides to about nineteen nucleotides wherein a bulge comprising from about one nucleotide to about two nucleotides is present in the first side of the stem, and a first side of a second stem comprising from about one nucleotide to about three nucleotides. The second polynucleotide comprises from about seventeen nucleotides to about forty seven nucleotides, wherein portions of the polynucleotide form a double-stranded RNA having the following features (5′ to 3′): a second side of the second stem comprising from about one nucleotide to about three nucleotides, a bulge comprising from about two nucleotides to about six nucleotides, a first side of a third stem comprising from about two nucleotides to about five nucleotides wherein a bulge comprising from about one nucleotide to about three nucleotides is present in the first side of the third stem, a terminal loop comprising from about two nucleotides to about six nucleotides, a second side of the third stem comprising from about two nucleotides to about five nucleotides, and a second side of the first stem comprising from about seven nucleotides to about nineteen nucleotides.

In regard to consensus site 24, the first polynucleotide preferably comprises sixteen nucleotides, wherein portions of the polynucleotide form a double-stranded RNA having the following features (5′ to 3′): a first side of a stem comprising thirteen nucleotides wherein a bulge comprising one nucleotide is present between the sixth and seventh nucleotides of the first side of the stem, and a first side of a second stem comprising two nucleotides. Preferably, the first polynucleotide comprises the sequence 5′-nnnnagnggnnnnnng-3′ (SEQ ID NO:171) (bolded nucleotides indicate preferred basepairing; n is any nucleotide). The second polynucleotide preferably comprises thirty one nucleotides, wherein portions of the polynucleotide form a double-stranded RNA having the following features (5′ to 3′): a second side of the second stem comprising two nucleotides, a bulge comprising four nucleotides, a first side of a third stem comprising three nucleotides wherein a bulge comprising two nucleotides is present between the first and second nucleotides of the first side of the third stem, a terminal loop comprising four nucleotides, a second side of the third stem comprising three nucleotides, and a second side of the first stem comprising thirteen nucleotides. Preferably, the second polynucleotide comprises the sequence 5′-anaccnnungcgaaggcnnnnnncuggnnnn-3′ (SEQ ID NO:172) (bolded nucleotides indicate preferred basepairing; n is any nucleotide). Consensus site 24 is depicted in FIG. 2.

Consensus site 25 comprises a region of RNA comprising a first and second polynucleotide. The first polynucleotide comprises from about five nucleotides to about fourteen nucleotides, wherein portions of the polynucleotide form a double-stranded RNA having the following features (5′ to 3′): a first side of a stem comprising from about three nucleotides to about nine nucleotides wherein a first side of an internal loop comprising from about two nucleotides to about five nucleotides is present in the first side of the stem. The second polynucleotide comprises from about six nucleotides to about eighteen nucleotides, wherein portions of the polynucleotide form a double-stranded RNA having the following features (5′ to 3′): a second side of the, stem comprising from about three nucleotides to about nine nucleotides wherein a second side of the internal loop comprising from about three nucleotides to about nine nucleotides is present in the second side of the stem.

In regard to consensus site 25, the first polynucleotide preferably comprises nine nucleotides, wherein portions of the polynucleotide form a double-stranded RNA having the following features (5′ to 3′): a first side of a stem comprising six nucleotides wherein a first side of an internal loop comprising three nucleotides is present between the third and fourth nucleotides of the first side of the stem. Preferably, the first polynucleotide comprises the sequence 5′-nguguagng-3′ (bolded nucleotides indicate preferred basepairing; n is any nucleotide). The second polynucleotide preferably comprises twelve nucleotides, wherein portions of the polynucleotide form a double-stranded RNA having the following features (5′ to 3′): a second side of the stem comprising six nucleotides wherein a second side of the internal loop comprising six nucleotides is present between the third and fourth nucleotides of the second side of the stem. Preferably, the second polynucleotide comprises the sequence 5′-uncgnaganaun-3′ (SEQ ID NO:173) (bolded nucleotides indicate preferred basepairing; n is any nucleotide). Consensus site 25 is depicted in FIG. 2. The molecular interaction site comprises a drug-binding pocket encompassing an area defined by 7 Å by 11 Å and is located in the major groove side of stem 23 immediately below the unpaired nucleotide G685.

Consensus site 26 comprises a region of RNA comprising a first and second polynucleotide. The first polynucleotide comprises from about eight nucleotides to about twenty three nucleotides, wherein portions of the polynucleotide form a double-stranded RNA having the following features (5′ to 3′): a first side of a stem comprising from about four nucleotides to about twelve nucleotides wherein a bulge comprising from about two nucleotides to about five nucleotides is present in the first side of the stem and wherein a first side of an internal loop comprising from about two nucleotides to about five nucleotides is present in the first side of the stem. The second polynucleotide comprises from about six nucleotides to about seventeen nucleotides, wherein portions of the polynucleotide form a double-stranded RNA having the following features (5′ to 3′): a second side of the stem comprising from about four nucleotides to about twelve nucleotides wherein a second side of the internal loop comprising from about two nucleotides to about five nucleotides is present in the second side of the stem.

In regard to consensus site 26, the first polynucleotide preferably comprises fourteen nucleotides, wherein portions of the polynucleotide form a double-stranded RNA having the following features (5′ to 3′): a first side of a stem comprising eight nucleotides wherein a bulge comprising three nucleotides is present between the third and fourth nucleotides of the first side of the stem and wherein a first side of an internal loop comprising three nucleotides is present between the fifth and sixth nucleotides of the first side of the stem. Preferably, the first polynucleotide comprises the sequence 5′-ugggnagcnaacag-3′ (SEQ ID NO:174) (bolded nucleotides indicate preferred basepairing; n is any nucleotide). The second polynucleotide preferably comprises eleven nucleotides, wherein portions of the polynucleotide form a double-stranded RNA having the following features (5′ to 3′): a second side of the stem comprising eight nucleotides wherein a second side of the internal loop comprising three nucleotides is present between the third and fourth nucleotides of the second side of the stem. Preferably, the second polynucleotide comprises the sequence 5′-cugguagucca-3′ (SEQ ID NO:175) (bolded nucleotides indicate preferred basepairing; n is any nucleotide). Consensus site 26 is depicted in FIG. 2.

Consensus site 27 comprises a region of RNA comprising a polynucleotide comprising from about nine nucleotides to about twenty three nucleotides, wherein portions of the polynucleotide form a double-stranded RNA having the following features (5′ to 3′): a first side of a stem comprising from about two nucleotides to about five nucleotides, a terminal loop comprising from about five nucleotides to about thirteen nucleotides, and a second side of the stem comprising from about two nucleotides to about five nucleotides.

In regard to consensus site 27, the polynucleotide preferably comprises fifteen nucleotides, wherein portions of the polynucleotide form a double-stranded RNA having the following features (5′ to 3′): a first side of a stem comprising three nucleotides, a terminal loop comprising nine nucleotides, and a second side of the stem comprising three nucleotides. Preferably, the polynucleotide comprises the sequence 5′-aggauuagauacccu-3′ (SEQ ID NO:176) (bolded nucleotides indicate preferred basepairing; n is any nucleotide). Consensus site 27 is depicted in FIG. 2.

Consensus site 28 comprises a region of RNA comprising a first and second polynucleotide. The first polynucleotide comprises from about eight nucleotides to about twenty one nucleotides, wherein portions of the polynucleotide form a double-stranded RNA having the following features (5′ to 3′): a first side of a stem comprising from about four nucleotides to about ten nucleotides wherein a first side of an internal loop comprising from about three nucleotides to about nine nucleotides is present in the first side of the stem, and a dangling region comprising from about one nucleotide to about two nucleotides. The second polynucleotide comprises from about seven nucleotides to about eighteen nucleotides, wherein portions of the polynucleotide form a double-stranded RNA having the following features (5′ to 3′): a dangling region comprising from about one nucleotide to about two nucleotides, and a second side of the stem comprising from about four nucleotides to about ten nucleotides wherein a second side of the internal loop comprising from about two nucleotides to about six nucleotides is present in the second side of the stem.

In regard to consensus site 28, the first polynucleotide preferably comprises fourteen nucleotides, wherein portions of the polynucleotide form a double-stranded RNA having the following features (5′ to 3′): a first side of a stem comprising seven nucleotides wherein a first side of an internal loop comprising six nucleotides is present between the third and fourth nucleotides of the first side of the stem, and a dangling region comprising one nucleotide. Preferably, the first polynucleotide comprises the sequence 5′-ggggaguacgnncg-3′ (SEQ ID NO:177) (bolded nucleotides indicate preferred basepairing; n is any nucleotide). The second polynucleotide preferably comprises twelve nucleotides, wherein portions of the polynucleotide form a double-stranded RNA having the following features (5′ to 3′): a dangling region comprising one nucleotide, and a second side of the stem comprising seven nucleotides wherein a second side of the internal loop comprising four nucleotides is present between the fourth and fifth nucleotides of the second side of the stem. Preferably, the second polynucleotide comprises the sequence 5′-agnnunaaacuc-3′ (SEQ ID NO:178) (bolded nucleotides indicate preferred basepairing; n is any nucleotide). Consensus site 28 is depicted in FIG. 2.

Consensus site 29 comprises a region of RNA comprising a first and second polynucleotide. The first polynucleotide comprises from about twenty two nucleotides to about fifty seven nucleotides, wherein portions of the polynucleotide form a double-stranded RNA having the following features (5′ to 3′): a first side of a first stem comprising from about four nucleotides to about ten nucleotides, a bulge comprising from about three nucleotides to about seven nucleotides, a first side of a second stem comprising from about two nucleotides to about five nucleotides, a terminal loop comprising from about four nucleotides to about twelve nucleotides, a second side of the second stem comprising from about two nucleotides to about five nucleotides, a bulge comprising from about five nucleotides to about thirteen nucleotides, and a first side of a third stem comprising from about two nucleotides to about five nucleotides. The second polynucleotide comprises from about nine nucleotides to about twenty two nucleotides, wherein portions of the polynucleotide form a double-stranded RNA having the following features (5′ to 3′): a second side of the third stem comprising from about two nucleotides to about five nucleotides, a bulge comprising from about two nucleotides to about five nucleotides, and a second side of the first stem comprising from about four nucleotides to about ten nucleotides wherein a bulge comprising from about one nucleotide to about two nucleotides is present in the second side of the first stem.

In regard to consensus site 29, the first polynucleotide preferably comprises thirty eight nucleotides, wherein portions of the polynucleotide form a double-stranded RNA having the following features (5′ to 3′): a first side of a first stem comprising seven nucleotides, a bulge comprising five nucleotides, a first side of a second stem comprising three nucleotides, a terminal loop comprising eight nucleotides, a second side of the second stem comprising three nucleotides, a bulge comprising nine nucleotides, and a first side of a third stem comprising three nucleotides. Preferably, the first polynucleotide comprises the sequence 5′-auguggnuuaauucgangnnacgcgnanaaccuuaccn-3′ (SEQ ID NO:179) (bolded nucleotides indicate preferred basepairing; n is any nucleotide). The second polynucleotide preferably comprises fourteen nucleotides, wherein portions of the polynucleotide form a double-stranded RNA having the following features (5′ to 3′): a second side of the third stem comprising three nucleotides, a bulge comprising three nucleotides, and a second side of the first stem comprising seven nucleotides wherein a bulge comprising one nucleotide is present between the second and third nucleotides of the second side of the first stem. Preferably, the second polynucleotide comprises the sequence 5′-ngggcuncacacnu-3′ (SEQ ID NO:180) (bolded nucleotides indicate preferred basepairing; n is any nucleotide). Consensus site 29 is depicted in FIG. 3.

Consensus site 30 comprises a region of RNA comprising a first, second and third polynucleotide. The first polynucleotide comprises from about nine nucleotides to about twenty four nucleotides, wherein portions of the polynucleotide form a double-stranded RNA having the following features (5′ to 3′): a first side of a first stem comprising from about two nucleotides to about five nucleotides, a bulge comprising from about three nucleotides to about seven nucleotides, and a first side of a second stem comprising from about four nucleotides to about twelve nucleotides. The second polynucleotide comprises from about nine nucleotides to about twenty three nucleotides, wherein portions of the polynucleotide form a double-stranded RNA having the following features (5′ to 3′): a second side of the second stem comprising from about four nucleotides to about twelve nucleotides wherein a bulge comprising from about one nucleotide to about two nucleotides is present in the second side of the second stem, a bulge comprising from about one nucleotide to about two nucleotides, and a first side of a third stem comprising from about two nucleotides to about five nucleotides wherein a bulge comprising from about one nucleotide to about two nucleotides is present in the first side of the third stem. The third polynucleotide comprises from about five nucleotides to about fourteen nucleotides, wherein portions of the polynucleotide form a double-stranded RNA having the following features (5′ to 3′): a second side of the third stem comprising from about one nucleotide to about three nucleotides, a bulge comprising from about two nucleotides to about six nucleotides, and a second side of the first stem comprising from about two nucleotides to about five nucleotides.

In regard to consensus site 30, the first polynucleotide preferably comprises sixteen nucleotides, wherein portions of the polynucleotide form a double-stranded RNA having the following features (5′ to 3′): a first side of a first stem comprising three nucleotides, a bulge comprising five nucleotides, and a first side of a second stem comprising eight nucleotides. Preferably, the first polynucleotide comprises the sequence 5′-nnnuugacaunnnnnn-3′ (SEQ ID NO:181) (bolded nucleotides indicate preferred basepairing; n is any nucleotide). The second polynucleotide preferably comprises fourteen nucleotides, wherein portions of the polynucleotide form a double-stranded RNA having the following features (5′ to 3′): a second side of the second stem comprising eight nucleotides wherein a bulge comprising one nucleotide is present between the fifth and sixth nucleotides of the second side of the second stem, a bulge comprising one nucleotide, and a first side of a third stem comprising three nucleotides wherein a bulge comprising one nucleotide is present between the second and third nucleotides of the first side of the third stem. Preferably, the second polynucleotide comprises the sequence 5′-nnnnnnnaacaggug-3′ (SEQ ID NO:182) (bolded nucleotides indicate preferred basepairing; n is any nucleotide). The third polynucleotide preferably comprises ten nucleotides, wherein portions of the polynucleotide form a double-stranded RNA having the following features (5′ to 3′): a second side of the third stem comprising two nucleotides, a bulge comprising four nucleotides, and a second side of the first stem comprising three nucleotides. Preferably, the third polynucleotide comprises the sequence 5′-cccuuangnn-3′ (SEQ ID NO:183) (bolded nucleotides indicate preferred basepairing; n is any nucleotide). Consensus site 30 is depicted in FIG. 3. The molecular interaction site comprises a drug-binding pocket encompassing an area defined by 15 Å by 7 Å and faces the major groove side of base-pair G993/C1045 and covers the 3-way junction between stems 32, 33 and 34.

Consensus site 31 comprises a region of RNA comprising a first and second polynucleotide. The first polynucleotide comprises from about eight nucleotides to about twenty two nucleotides, wherein portions of the polynucleotide form a double-stranded RNA having the following features (5′ to 3′): a first side of a stem comprising from about five nucleotides to about fifteen nucleotides wherein a bulge comprising from about one nucleotide to about two nucleotides is present in the first side of the stem and wherein a first side of an internal loop comprising from about two nucleotides to about five nucleotides is present in the first side of the stem. The second polynucleotide comprises from about eight nucleotides to about twenty two nucleotides, wherein portions of the polynucleotide form a double-stranded RNA having the following features (5′ to 3′): a second side of the stem comprising from about five nucleotides to about fifteen nucleotides wherein a bulge comprising from about two nucleotides to about five nucleotides is present in the second side of the stem and wherein a second side of the internal loop comprising from about one nucleotide to about two nucleotides is present in the second side of the stem.

In regard to consensus site 31, the first polynucleotide preferably comprises fourteen nucleotides, wherein portions of the polynucleotide form a double-stranded RNA having the following features (5′ to 3′): a first side of a stem comprising ten nucleotides wherein a bulge comprising one nucleotide is present between the second and third nucleotides of the first side of the stem and wherein a first side of an internal loop comprising three nucleotides is present between the fifth and sixth nucleotides of the first side of the stem. Preferably, the first polynucleotide comprises the sequence 5′-ggugnugcauggnu-3′ (SEQ ID NO:184) (bolded nucleotides indicate preferred basepairing; n is any nucleotide). The second polynucleotide preferably comprises fourteen nucleotides, wherein portions of the polynucleotide form a double-stranded RNA having the following features (5′ to 3′): a second side of the stem comprising ten nucleotides wherein a bulge comprising three nucleotides is present between the third and fourth nucleotides of the second side of the stem and wherein a second side of the internal loop comprising one nucleotide is present between the fifth and sixth nucleotides of the second side of the stem. Preferably, the second polynucleotide comprises the sequence 5′-anucnucaugnccc-3′ (SEQ ID NO:185) (bolded nucleotides indicate preferred basepairing; n is any nucleotide): Consensus site 31 is depicted in FIG. 3. The molecular interaction site comprises two drug-binding pockets located in the major groove of stem 34, on either side of this motif and encompassing an area defmed by 10 Å by 10 Å (upper pocket) and faces the sugar edge of G1053 and 13 Å by 13 Å (lower pocket) and is centered around the base-pairG1050/C1208.

Consensus site 32 comprises a region of RNA comprising a first and second polynucleotide. The first polynucleotide comprises from about seven nucleotides to about eighteen nucleotides, wherein portions of the polynucleotide form a double-stranded RNA having the following features (5′ to 3′): a first side of a stem comprising from about five nucleotides to about thirteen nucleotides wherein a first side of an internal loop comprising from about two nucleotides to about five nucleotides is present in the first side of the stem. The second polynucleotide comprises from about nine nucleotides to about twenty two nucleotides, wherein portions of the polynucleotide form a double-stranded RNA having the following features (5′ to 3′): a second side of the stem comprising from about five nucleotides to about thirteen nucleotides wherein a bulge comprising from about one nucleotide to about two nucleotides is present in the second side of the stem and wherein a bulge comprising from about two nucleotides to about five nucleotides is present in the second side of the stem and wherein a second side of the internal loop comprising from about one nucleotide to about two nucleotides is present in the second side of the stem.

In regard to consensus site 32, the first polynucleotide preferably comprises twelve nucleotides, wherein portions of the polynucleotide form a double-stranded RNA having the following features (5′ to 3′): a first side of a stem comprising nine nucleotides wherein a first side of an internal loop comprising three nucleotides is present between the first and second nucleotides of the first side of the stem. Preferably, the first polynucleotide comprises the sequence 5′-ugcauggnuguc-3′ (SEQ ID NO:186) (bolded nucleotides indicate preferred basepairing; n is any nucleotide). The second polynucleotide preferably comprises fourteen nucleotides, wherein portions of the polynucleotide form a double-stranded RNA having the following features (5′ to 3′): a second side of the stem comprising nine nucleotides wherein a bulge comprising one nucleotide is present between the third and fourth nucleotides of the second side of the stem and wherein a bulge comprising three nucleotides is present between the sixth and seventh nucleotides of the second side of the stem and wherein a second side of the internal loop comprising one nucleotide is present between the eighth and ninth nucleotides of the second side of the stem. Preferably, the second polynucleotide comprises the sequence 5′-gucaanucnucaug-3′ (SEQ ID NO:187) (bolded nucleotides indicate preferred basepairing; n is any nucleotide). Consensus site 32 is depicted in FIG. 3.

Consensus site 33 comprises a region of RNA comprising a first and second polynucleotide. The first polynucleotide comprises from about thirty nucleotides to about eighty four nucleotides, wherein portions of the polynucleotide form a double-stranded RNA having the following features (5′ to 3′): a first side of a first stem comprising from about two nucleotides to about five nucleotides, a bulge comprising from about two nucleotides to about six nucleotides, a first side of a second stem comprising from about three nucleotides to about nine nucleotides, a first side of a third stem comprising from about two nucleotides to about five nucleotides, a first terminal loop comprising from about two nucleotides to about six nucleotides; a second side of the third stem comprising from about two nucleotides to about five nucleotides, a bulge comprising from about one nucleotide to about three nucleotides, a first side of a fourth stem comprising from about two nucleotides to about six nucleotides, a second terminal loop comprising from about three nucleotides to about nine nucleotides, a second side of the fourth stem comprising from about two nucleotides to about six. nucleotides, a bulge comprising from about one nucleotide to about three nucleotides, a second side of the second stem comprising from about three nucleotides to about nine nucleotides, a bulge comprising from about three nucleotides to about seven nucleotides, and a first side of a fifth stem comprising from about two nucleotides to about five nucleotides. The second polynucleotide comprises from about seven nucleotides to about seventeen nucleotides, wherein portions of the polynucleotide form a double-stranded RNA having the following features (5′ to 3′): a second side of the fifth stem comprising from about two nucleotides to about five nucleotides, a bulge comprising from about three nucleotides to about seven nucleotides, and a second side of the first stem comprising from about two nucleotides to about five nucleotides.

In regard to consensus site 33, the first polynucleotide preferably comprises fifty five nucleotides, wherein portions of the polynucleotide form a double-stranded RNA having the following features (5′ to 3′): a first side of a first stem comprising three nucleotides, a bulge comprising four nucleotides, a first side of a second stem comprising six nucleotides, a first side of a third stem comprising three nucleotides, a first terminal loop comprising four nucleotides, a second side of the third stem comprising three nucleotides, a bulge comprising two nucleotides, a first side of a fourth stem comprising four nucleotides, a second terminal loop comprising six nucleotides, a second side of the fourth stem comprising four nucleotides, a bulge comprising two nucleotides, a second side of the second stem comprising six nucleotides, a bulge comprising five nucleotides, and a first side of a fifth stem comprising three nucleotides. Preferably, the first polynucleotide comprises the sequence 5′-gucgucagcucgugnngugannuguuggguuaagucccgnaacgagcgcaacccn-3′ (SEQ ID NO:188) (bolded nucleotides indicate preferred basepairing; n is any nucleotide). The second polynucleotide preferably comprises eleven nucleotides, wherein portions of the polynucleotide form a double-stranded RNA having the following features (5′ to 3′): a second side of the fifth stem comprising three nucleotides, a bulge comprising five nucleotides, and a second side of the first stem comprising three nucleotides. Preferably, the second polynucleotide comprises the sequence 5′-gggangacguc-3′ (SEQ ID NO:189) (bolded nucleotides indicate preferred basepairing; n is any nucleotide). Consensus site 33 is depicted in FIG. 3. The molecular interaction site comprises a drug-binding pocket encompassing an area defined by 13 Å by 13 Å and formed by the minor groove of stem 34 and extends to the sugar rings of nucleotides G1068 and C1069.

Consensus site 34 comprises a region of RNA comprising a first, second and third polynucleotide. The first polynucleotide comprises from about four nucleotides to about eleven nucleotides, wherein portions of the polynucleotide form a double-stranded RNA having the following features (5′ to 3′): a first side of a first stem comprising from about three nucleotides to about nine nucleotides wherein a bulge comprising from about one nucleotide to about two nucleotides is present in the first side of the first stem. The second polynucleotide comprises from about seven nucleotides to about twelve nucleotides, wherein portions of the polynucleotide form a double-stranded RNA having the following features (5′ to 3′): two nucleotides basepairing with the last two nucleotides of the first side of the first stem forming part of the second side of the first stem, a bulge comprising from about one nucleotide to about three nucleotides, and a first side of a second stem comprising from about three nucleotides to about seven nucleotides wherein a first side of an internal loop comprising from about one nucleotide to about two nucleotides is present in the first side of the second stem. The third polynucleotide comprises from about eleven nucleotides to about sixteen nucleotides, wherein portions of the polynucleotide form a double-stranded RNA having the following features (5′ to 3′): a second side of the second stem comprising from about three nucleotides to about seven nucleotides wherein a second side of the internal loop comprising from about one nucleotide to about two nucleotides is present in the second side of the second stem, a bulge comprising from about three nucleotides to about seven nucleotides, and four nucleotides basepairing with the first four nucleotides of the first side of the first stem forming part of the second side of the first stem.

In regard to consensus site 34, the first polynucleotide preferably comprises seven nucleotides, wherein portions of the polynucleotide form a double-stranded RNA having the following features (5′ to 3′): a first side of a first stem comprising six nucleotides wherein a bulge comprising one nucleotide is present between the fourth and fifth nucleotides of the first side of the first stem. Preferably, the first polynucleotide comprises the sequence 5′-ccnnnnn-3′ (bolded nucleotides indicate preferred basepairing; n is any nucleotide). The second polynucleotide preferably comprises ten nucleotides, wherein portions of the polynucleotide form a double-stranded RNA having the following features (5′ to 3′): two nucleotides basepairing with the last two nucleotides of the first side of the first stem forming part of the second side of the first stem, a bulge comprising two nucleotides, and a first side of a second stem comprising five nucleotides wherein a first side of an internal loop comprising one nucleotide is present between the first and second nucleotides of the first side of the second stem. Preferably, the second polynucleotide comprises the sequence 5′-nnnacugccn-3′ (SEQ ID NO:190) (bolded nucleotides indicate preferred basepairing; n is any nucleotide). The third polynucleotide preferably comprises fifteen nucleotides, wherein portions of the polynucleotide form a double-stranded RNA having the following features (5′ to 3′): a second side of the second stem comprising five nucleotides wherein a second side of the internal loop comprising one nucleotide is present between the fourth and fifth nucleotides of the second side of the second stem, a bulge comprising five nucleotides, and four nucleotides basepairing with the first four nucleotides of the first side of the first stem forming part of the second side of the first stem. Preferably, the third polynucleotide comprises the sequence 5′-nggaggaaggngggg-3′ (SEQ ID NO:191) (bolded nucleotides indicate preferred basepairing; n is any nucleotide). Consensus site 34 is depicted in FIG. 3.

Consensus site 35 comprises a region of RNA comprising a first and second polynucleotide. The first polynucleotide comprises from about seven nucleotides to about eighteen nucleotides, wherein portions of the polynucleotide form a double-stranded RNA having the following features (5′ to 3′): a first side of a stem comprising from about four nucleotides to about ten nucleotides wherein a first side of an internal loop comprising from about two nucleotides to about five nucleotides is present in the first side of the stem and wherein a bulge comprising from about one nucleotide to about three nucleotides is present in the first side of the stem. The second polynucleotide comprises from about six nucleotides to about sixteen nucleotides, wherein portions of the polynucleotide form a double-stranded RNA having the following features (5′ to 3′): a second side of the stem comprising from about four nucleotides to about ten nucleotides wherein a second side of the internal loop comprising from about two nucleotides to about six nucleotides is present in the second side of the stem.

In regard to consensus site 35, the first polynucleotide preferably comprises twelve nucleotides, wherein portions of the polynucleotide form a double-stranded RNA having the following features (5′ to 3′): a first side of a stem comprising seven nucleotides wherein a first side of an internal loop comprising three nucleotides is present between the fourth and fifth nucleotides of the first side of the stem and wherein a bulge comprising two nucleotides is present between the sixth and seventh nucleotides of the first side of the stem. Preferably, the first polynucleotide comprises the sequence 5′-nnnguuncnanc-3′ (SEQ ID NO:192) (bolded nucleotides indicate preferred basepairing; n is any nucleotide). The second polynucleotide preferably comprises eleven nucleotides, wherein portions of the polynucleotide form a double-stranded RNA having the following features (5′ to 3′): a second side of the stem comprising seven nucleotides wherein a second side of the internal loop comprising four nucleotides is present between the third and fourth nucleotides of the second side of the stem. Preferably, the second polynucleotide comprises the sequence 5′-gngnacucnnn-3′ (SEQ ID NO:193) (bolded nucleotides indicate preferred basepairing; n is any nucleotide). Consensus site 35 is depicted in FIG. 3.

Consensus site 36 comprises a region of RNA comprising a first and second polynucleotide. The first polynucleotide comprises from about six nucleotides to about fourteen nucleotides, wherein portions of the polynucleotide form a double-stranded RNA having the following features (5′ to 3′): a first side of a stem comprising from about three nucleotides to about seven nucleotides wherein a first side of an internal loop comprising from about three nucleotides to about seven nucleotides is present in the first side of the stem. The second polynucleotide comprises from about five nucleotides to about fourteen nucleotides, wherein portions of the polynucleotide form a double-stranded RNA having the following features (5′ to 3′): a second side of the stem comprising from about three nucleotides to about seven nucleotides wherein a second side of the internal loop comprising from about two nucleotides to about seven nucleotides is present in the second side of the stem.

In regard to consensus site 36, the first polynucleotide preferably comprises ten nucleotides, wherein portions of the polynucleotide form a double-stranded RNA having the following features (5′ to 3′): a first side of a stem comprising five nucleotides wherein a first side of an internal loop comprising five nucleotides is present between the second and third nucleotides of the first side of the stem. Preferably, the first polynucleotide comprises the sequence 5′-nnacanngng-3′ (SEQ ID NO:194) (bolded nucleotides indicate preferred basepairing; n is any nucleotide). The second polynucleotide preferably comprises nine or ten nucleotides, wherein portions of the polynucleotide form a double-stranded RNA having the following features (5′ to 3′): a second side of the stem comprising five nucleotides wherein a second side of the internal loop comprising four or five nucleotides is present between the third and fourth nucleotides of the second side of the stem. Preferably, the second polynucleotide comprises the sequence 5′-cnnnaaann-3′ or 5′-cnnnnaaann-3′ (SEQ ID NO:195) (bolded nucleotides indicate preferred basepairing; n is any nucleotide). Consensus site 36 is depicted in FIG. 3.

Consensus site 37 comprises a region of RNA comprising a polynucleotide comprising from about seventeen nucleotides to about forty nine nucleotides, wherein portions of the polynucleotide form a double-stranded RNA having the following features (5′ to 3′): a first side of a stem comprising from about four nucleotides to about twelve nucleotides wherein a first side of a first internal loop comprising from about one nucleotide to about three nucleotides is present in the first side of the stem and wherein a first side of a second internal loop comprising from about two nucleotides to about five nucleotides is present in the first side of the stem, a terminal loop comprising from about two nucleotides to about six nucleotides, and a second side of the stem comprising from about four nucleotides to about twelve nucleotides wherein a second side of the second internal loop comprising from about two nucleotides to about five nucleotides is present in the second side of the stem and wherein a second side of the first internal loop comprising from about two nucleotides to about six nucleotides is present in the second side of the stem.

In regard to consensus site 37, the polynucleotide preferably comprises thirty two nucleotides, wherein portions of the polynucleotide form a double-stranded RNA having the following features (5′ to 3′): a first side of a stem comprising eight nucleotides wherein a first side of a first internal loop comprising two nucleotides is present between the third and fourth nucleotides of the first side of the stem and wherein a first side of a second internal loop comprising three nucleotides is present between the fifth and sixth nucleotides of the first side of the stem, a terminal loop comprising four nucleotides, and a second side of the stem comprising eight nucleotides wherein a second side of the second internal loop comprising three nucleotides is present between the third and fourth nucleotides of the second side of the stem and wherein a second side of the first internal loop comprising four nucleotides is present between the fifth and sixth nucleotides of the second side of the stem. Preferably, the polynucleotide comprises the sequence 5′-gngnngcnannnngnnannnnnagcnaancnn-3′ (SEQ ID NO:196) (bolded nucleotides indicate preferred basepairing; n is any nucleotide). Consensus site 37 is depicted in FIG. 3.

Consensus site, 38 comprises a region of RNA comprising a polynucleotide comprising from about eight nucleotides to about twenty two nucleotides, wherein portions of the polynucleotide form a double-stranded RNA having the following features (5′ to 3′): a first side of a stem comprising from about two nucleotides to about five nucleotides, a terminal loop comprising from about four nucleotides to about twelve nucleotides, and a second side of the stem comprising from about two nucleotides to about five nucleotides.

In regard to consensus site 38, the polynucleotide preferably comprises fourteen nucleotides, wherein portions of the polynucleotide form a double-stranded RNA having the following features (5′ to 3′): a first side of a stem comprising three nucleotides, a terminal loop comprising eight nucleotides, and a second side of the stem comprising three nucleotides. Preferably, the polynucleotide comprises the sequence 5′-nncugcaacucgnn-3′ (SEQ ID NO:197) (bolded nucleotides indicate preferred basepairing; n is any nucleotide). Consensus site 38 is depicted in FIG. 3.

Consensus site 39 comprises a region of RNA comprising a polynucleotide comprising from about eight nucleotides to about twenty nucleotides, wherein portions of the polynucleotide form a double-stranded RNA having the following features (5′ to 3′): a first side of a stem comprising from about two nucleotides to about five nucleotides, a terminal loop comprising from about four nucleotides to about ten nucleotides, and a second side of the stem comprising from about two nucleotides to about five nucleotides.

In regard to consensus site 39, the polynucleotide preferably comprises thirteen nucleotides, wherein portions of the polynucleotide form a double-stranded RNA having the following features (5′ to 3′): a first side of a stem comprising three nucleotides, a terminal loop comprising seven nucleotides, and a second side of the stem comprising three nucleotides. Preferably, the polynucleotide comprises the sequence 5′-nnaucagnangnn-3′ (SEQ ID NO:198) (bolded nucleotides indicate preferred basepairing; n is any nucleotide). Consensus site 39 is depicted in FIG. 3.

Consensus site 40 comprises a region of RNA comprising a first and second polynucleotide. The first polynucleotide comprises from about five nucleotides to about thirteen nucleotides, wherein portions of the polynucleotide form a double-stranded RNA having the following features (5′ to 3′): a first side of a stem comprising from about three nucleotides to about nine nucleotides wherein a first side of a first internal loop comprising from about one nucleotide to about two nucleotides is present in the first side of the stem and wherein a first side of a second internal loop comprising from about one nucleotide to about two nucleotides is present in the first side of the stem. The second polynucleotide comprises from about five nucleotides to about fourteen nucleotides, wherein portions of the polynucleotide form a double-stranded RNA having the following features (5′ to 3′): a second side of the stem comprising from about three nucleotides to about nine nucleotides wherein a second side of the first internal loop comprising from about one nucleotide to about three nucleotides is present in the second side of the stem and wherein a second side of the second internal loop comprising from about one nucleotide to about two nucleotides is present in-the second side of the stem.

In regard to consensus site 40, the first polynucleotide preferably comprises eight nucleotides, wherein portions of the polynucleotide form a double-stranded RNA having the following features (5′ to 3′): a first side of a stem comprising six nucleotides wherein a first side of a first internal loop comprising one nucleotide is present between the first and second nucleotides of the first side of the stem and wherein a first side of a second internal loop comprising one nucleotide is present between the second and third nucleotides of the first side of the stem. Preferably, the first polynucleotide comprises the sequence 5′-gucannnc-3′ (bolded nucleotides indicate preferred basepairing; n is any nucleotide). The second polynucleotide preferably comprises nine nucleotides, wherein portions of the polynucleotide form a double-stranded RNA having the following features (5′ to 3′): a second side of the stem comprising six nucleotides wherein a second side of the first internal loop comprising two nucleotides is present between the fourth and fifth nucleotides of the second side of the stem and wherein a second side of the second internal loop comprising one nucleotide is present between the fifth and sixth nucleotides of the second side of the stem. Preferably, the second polynucleotide comprises the sequence 5′-gnnnaaguc-3′ (bolded nucleotides indicate preferred basepairing; n is any nucleotide). Consensus site 40 is depicted in FIG. 4.

The molecular interaction site comprises a drug-binding pocket encompassing an area defined by 12 Å by 13 Å and is located in the major groove of stem 44 around the nucleotides G1491 and C1408.

Consensus site 41 comprises a region of RNA comprising a first and second polynucleotide. The first polynucleotide comprises from about five nucleotides to about fifteen nucleotides, wherein portions of the polynucleotide form a double-stranded RNA having the following features (5′ to 3′): a first side of a stem comprising from about four nucleotides to about twelve nucleotides wherein a first side of an internal loop comprising from about one nucleotide to about three nucleotides is present in the first side of the stem. The second polynucleotide comprises from about five nucleotides to about fifteen nucleotides, wherein portions of the polynucleotide form a double-stranded RNA having the following features (5′ to 3′): a second side of the stem comprising from about four nucleotides to about twelve nucleotides wherein a second side of the internal loop comprising from about one nucleotide to about three nucleotides is present in the second side of the stem.

In regard to consensus site 41, the first polynucleotide preferably comprises ten nucleotides, wherein portions of the polynucleotide form a double-stranded RNA having the following features (5′ to 3′): a first side of a stem comprising eight nucleotides wherein a first side of an internal loop comprising two nucleotides is present between the fifth and sixth nucleotides of the first side of the stem. Preferably, the first polynucleotide comprises the sequence 5′-cangnnagnn-3′ (SEQ ID NO:199) (bolded nucleotides indicate preferred basepairing; n is any nucleotide). The second polynucleotide preferably comprises ten nucleotides, wherein portions of the polynucleotide form a double-stranded RNA having the following features (5′ to 3′): a second side of the stem comprising eight nucleotides wherein a second side of the internal loop comprising two nucleotides is present between the third and fourth nucleotides of the second side of the stem. Preferably, the second polynucleotide.comprises the sequence 5′-nnnganuggg-3′ (SEQ ID NO:200) (bolded nucleotides indicate preferred basepairing; n is any nucleotide). Consensus site 41 is depicted in FIG. 4. The molecular interaction site comprises a drug-binding pocket encompassing an area defined by 13 Å by 15 Å and is located in the major groove side of stem 44 and is centered around the nucleotides G1417 and G1482.

Consensus site 42 comprises a region of RNA comprising a first and second polynucleotide. The first polynucleotide comprises from about five nucleotides to about thirteen nucleotides, wherein portions of the polynucleotide form a double-stranded RNA having the following features (5′ to 3′): a dangling region comprising from about one nucleotide to about three nucleotides, a first side of a stem comprising from about two nucleotides to about six nucleotides wherein a first side of a first internal loop comprising from about one nucleotide to about two nucleotides is present in the first side of the stem, and a dangling region comprising from about one nucleotide to about two nucleotides. The second polynucleotide comprises from about twenty nucleotides to about fifty three nucleotides, wherein portions of the polynucleotide form a double-stranded RNA having the following features (5′ to 3′): a dangling region comprising from about one nucleotide to about two nucleotides, a second side of the stem comprising from about two nucleotides to about six nucleotides wherein a second side of the first internal loop comprising from about one nucleotide to about three nucleotides is present in the second side of the stem, a bulge comprising from about two nucleotides to about six nucleotides, a first side of a second stem comprising from about five nucleotides to about thirteen nucleotides wherein a first side of a second internal loop comprising from about one nucleotide to about two nucleotides is optionally present in the first side of the second stem, a terminal loop comprising from about two nucleotides to about six nucleotides, and a second side of the second stem comprising from about five nucleotides to about thirteen nucleotides wherein a bulge or second side of the second internal loop comprising from about one nucleotide to about two nucleotides is present in the second side of the second stem.

In regard to consensus site 42, the first polynucleotide preferably comprises eight nucleotides, wherein portions of the polynucleotide form a double-stranded RNA having the following features (5′ to 3′): a dangling region comprising two nucleotides, a first side of a stem comprising four nucleotides wherein a first side of a first internal loop comprising one nucleotide is present between the second and third nucleotides of the first side of the stem, and a dangling region comprising one nucleotide. Preferably, the first polynucleotide comprises the sequence 5′-ccgcccgu-3′ (bolded nucleotides indicate preferred basepairing; n is any nucleotide). The second polynucleotide preferably comprises thirty four or thirty five nucleotides, wherein portions of the polynucleotide form a double-stranded RNA having the following features (5′ to 3′): a dangling region comprising one nucleotide, a second side of the stem comprising four nucleotides wherein a second side of the first internal loop comprising two nucleotides is present between the third and fourth nucleotides of the second side of the stem, a bulge comprising four nucleotides, a first side of a second stem comprising nine nucleotides wherein a first side of a second internal loop comprising one nucleotide is optionally present between the fifth and sixth nucleotides of the first side of the second stem, a terminal loop comprising four nucleotides, and a second side of the second stem comprising nine nucleotides wherein a bulge or second side of the second internal loop comprising one nucleotide is present between the fourth and fifth nucleotides of the second side of the second stem. Preferably, the second polynucleotide comprises the sequence 5′-ucguaacaagguanccuannngaannugnggnug-3′ (SEQ ID NO:201) or 5′-ucguaacaagguanccnuannngaannugnggnug-3′ (SEQ ID NO:202) (bolded nucleotides indicate preferred basepairing; n is any nucleotide). Consensus site 42 is depicted in FIG. 4.

### Example 3

### The Bacterial 16S rRNA 690 Loop (Site 25)

In considering subdomain targets in the bacterial 16S rRNA, site 25 meets the criteria for a good subdomain for drug discovery. A substantial portion of the nucleotides in the 690 loop region are broadly conserved across different bacterial taxonomic species. Over 6,000 different sequences of bacteria were considered. For examination of the status of each nucleotide, nucleotide 680 is paired with nucleotide 710. In the case of the 680-710 pair, 90.32% of the time the pair in this position is a C-G pair. This means that both the identities of the nucleotides and the fact that they are paired is very conserved. However, in the relatively few (<10%) of the cases where this pair is not a C-G, another pair occurs in its place—g-c (4.24%), u-a (3.11%), and a-u (1.4%). This means that in the few cases where a C-G pair does not occur in this position, some other pair replaces it and, thus, the paired structure is preserved.

Moving to the next pair (walking the subdomain from the stem portion to the terminal loop), the nucleotide pair 681-709 is an A-U pair, which is derived from the exact sequence from E. coli. However, in all the bacteria considered, a u-a (41.14%) occurs more frequently than a-u (31.33%), followed by c-g (16.14%) and g-c (10.49%). While this distribution of different nucleotide identities is greater that in the previous example, we find that there is extreme conservation (>99%) of some base pair in this position. This indicates that, while there is not great pressure to maintain a particular nucleotide in positions 681 or 709, there is pressure to maintain pairing partners, which is indicative of conservative of structure. This situation is considered an example of structure conservation, without sequence conservation. The next pair, 682-708 is similar, where structure conservation, without sequence conservation is also observed. The next two positions show a high degree of both sequence and structure conservation. Positions 683-708 is a G-U pair, rather than a typical Watson-Crick pair, followed by a Watson-Crick U-A pair. Non-canonical (other than Watson-Crick) pairs occur frequently in RNA structures, and thus, base pairing is not limited to Watson-Crick pairs in the present invention. Thus, this base paired region (from 680-684 and 706-710) is very conserved in bacteria with respect to structure and partially conserved with respect to nucleotide sequence identity. This region is considered to be exemplary of a conserved subdomain, where drugs that bind will have the opportunity to provide very broad spectrum anti-bacterial activity.

To determine which bacterial species (or which groups of bacteria) might have slight variations in the 690 subdomain, the sequences of many of the most important human pathogens were examined and organized into a bacterial phylogeny. The variations were determined to cluster along the lines of phylogeny. For example, the Actinobacteria tend to have substitutions in both 694A to G and 701U to G variations. In a drug discovery effort using the 690 subdomain, a sequence that has these substitutions can be examined in order to determine if a drug candidate will have activity that includes these important human pathogens.

The next issue in selection of a subdomain is the opportunity for a therapeutic index. Human beings have an analogous structure to the bacterial 690 structure in both the nuclear-encoded and mitochondrial-encoded rRNAs. There are a significant number of changes required to go from the bacterial to the human nuclear structure, and even more changes are required to go to the human mitochondrial structure. Thus, there is an excellent chance that a drug that binds specifically to the bacterial target will not bind as tightly to the human target, and thus have a greater potential for lower toxicity. It is noteworthy that it is not absolutely essential to have a large number of differences between the bacterial and human targets. Selectivity for the bacteria can be achieved in ways other than having differences in the target.

### Example 4

### Decoding Region-Related Structures

A large body evidence supports the hypothesis that the decoding region is the primary docking site (or receptor) for the two adjacent A- and P-site codon—anticodon mini-helices. Consistent with this hypothesis, the decoding region may be considered to be structurally and functionally subdividable into adjacent A- and P-site subdomains. When stabilized by heterologous “clamp” and “tetraloop terminator” structures in polynucleotide analogs, these sequences appear to fold and function similarly to the way they do in intact ribosomes. In addition, an A-site subdomain analog binds neomycin-like aminoglycoside antibiotics and has been characterized by NMR. In 16S rRNA, the proximal end of the decoding region is closely associated with 5′-pseudoknot nucleotides 14 and 15 (via tertiary interaction) and the G926 helix. The proximal end of the decoding region can stack onto the two base pair helical tertiary interaction (14:1398, 15:1397), which, in turn, continues the adjacent G926 Helix. These stacking interactions appear to form a “dynamic clamping system” at the proximal end of the decoding region, which may change conformation during ribosome function. Unstacking of the system would presumably destabilize the quasi-helical conformation of the decoding region, which is by itself quite unstable. Helical unstacking may be induced by conformational changes involving U14 and G15, nucleotides in the 5′-pseudoknot loop. These bases may, in turn, transmit conformational changes occurring in other parts of the pseudoknot and/or in the closely associated 900 Stem-Loop, or Central Switch, region. This mechanism may explain how association of streptomycin and tetracycline with the Central Switch region affects A-site function. The Central Switch Region (900 Stem-Loop).

A large body of evidence supports the hypothesis that the Central Switch region is involved in. adjusting the efficiency/accuracy of the A-site. First, the region is thought to interact with tetracycline and streptomycin: tetracycline is a classic A-site blocker, while streptomycin is the classic miss-coding inducing aminoglycoside (miss-coding is an A-site-associated activity). Second, numerous nucleotides in this region respond to assembly of proteins S5 and S12, both of which are involved in modulating the ribosome's response to aminoglycoside-induced miss-coding. Interestingly, almost all of these positions respond to either S5 or S12, suggesting that their protection is due to a protein-induced conformational change, rather than direct binding. Consistent with this hypothesis, the region is devoid of any protein-induced backbone protections. The presumed conformational flexibility of this region is supported by the recent discovery of a “conformational switch” within it. Despite their participation in A-site function, nucleotides in this region do not appear to directly interact with tRNA or mRNA. Rather, as noted above, the Central Switch region appears to exert its affects via its close association with the 5′-pseudoknot helical system, which may in turn control the decoding region. The proximal end of the 900 Stem-Loop is stacked onto the adjacent 566-570/880-884 helix, and joined to the pseudoknot helical system via the streptomycin binding site (A913-A915) and a long single-strand (G557-U565). Two Central Switch Analogs, including, and not including, the closely associated pseudoknot helical system are present. The first, Pseudoknot Form incorporates two tetraloop terminations: one, replacing the central domain of 16S rRNA, stabilizes the 566-570/880-884 helix, and the other one, replacing the 5′-domain, terminates the pseudoknot helical system. This analog may contain intact tetracycline and streptomycin binding sites. It offers bacteria-specific targeting potential via the E. coli-specific A19-U916 base pair, U884, C896, and a large group of bases at the distal end of the pseudoknot and in the 560 connecting strand. The second, simpler, Clamp Form analog omits the pseudoknot system, and offers only U884 and C896 as E. coli-specific targets.

### Example 5

### Inter-Bacterial Targeting

Phylogenetic sequence variations may facilitate not only differential human host-bacterial targeting (as described above), but also inter-bacterial targeting. In either case, to avoid damage to the human host, sequence-specific RNA-binding drugs should be designed to “see” only those nucleotides that specify bacterial rRNA sequences, since they will be functioning within a “background” of human host cytoplasmic and mitochondrial RNA sequences. Nucleotide substitutions in the Decoding Region and Central Switch analogs produced by H. influenzae (proteobacteria, gamma), B. subtilis (gram positive, low GC), and S. aureus (gram positive, low GC) sequences relative to E. coli, for example, can be used to develop small molecules that distinguish among these bacteria. The Decoding Region Form 1 analogs will support only limited inter-bacterial targeting, since the only sequence variations occur with S. aureus at (base paired) positions 931 and 1388. Thus, this analysis suggests that drugs targeting the (S. aureus) U and A at these two positions may specifically act on S. aureus, while drugs targeting the other positions in the analog will act as broad spectrum antibacterial compounds. In addition, drugs targeting (base paired) positions 19 and 916 should not be designed to act against either B. subtlis or S. aureus (and possibly other gram positive, low GC bacteria), since these positions are no longer bacteria-specific in these organisms. In the case of Central Switch analogs, Form 1 analogs may support extensive inter-bacterial targeting since H. influenzae, B. subtilis, and S. aureus offer additional bacteria-specific nucleotides (H. influenzae: 562, 564, B. subtilis: 903, 564, and S. aureus: 564). However, drugs targeting positions 19, 896, nor 916, should not be designed to act against B. subtilis nor S. aureus since these positions loose bacterial-specificity in these organisms. Finally, Central Switch Form 2 analogs may support selective targeting of B. subtilis as it offers an additional bacteria-specific nucleotide at position 903. In contrast, position 896 should not be targeted since it looses bacterial-specificity in this organism.

### Example 6

### The 530 Stem-Loop

The 530 loop is the only 16S rRNA segment outside of the decoding region containing adjacent A- and P-site-tRNA associated nucleotides. Interestingly, two of these nucleotides have also been crosslinked to mRNA positions +11 and +12, which are somewhat distant from the A- and P-site codons themselves (mRNA positions 1-6). The 530 Stem-Loop is endowed with a complement of pseudoknot-like tertiary interactions, which take the form of very short, G-C-rich helices. It remains unclear whether formation of these structures is mutually exclusive, or whether the region adopts unusual three-dimensional conformation(s) to accommodate them simultaneously. The presence of i) several streptomycin resistance mutations, and ii) a neomycin-mediated reactivity enhancement within these structures suggests that their formation may be related to the effects of these antibiotics, which are thought to bind elsewhere. Two “functionally organized” forms allow simultaneous formation of the tertiary interactions. In Form 1, the two tertiary helices stack on each other, while in Form 2, they stack onto the other, presumably stable helices of the 530 stem. Interestingly, in the Form 2 structure, the net result of the complex folding pattern appears to be an effective two base pair extension of the second stem helix; with the two single-stranded loop regions (the 520- and 530-strands) connecting the helix ends in circular fashion. This is, perhaps easier to see in the proposed analog structure, which would need to be built with two covalently closed circular RNA's. With the exception of U508, E. coli-specific nucleotides are found only in the two stable stems. The analog offers limited bacteria-specific targeting potential, via the E. coli-specific C513-G538 base pair.

Nucleotide substitutions exist in the 530 Stem-Loop for H. influenzae (proteobacteria, gamma), B. subtlis (gram positive, low GC), and S. aureus (gram positive, low GC) sequences. Analogs composed of the full 530 Stem-Loop sequence may support differentiation of proteobacteria gamma and gram positive, low GC bacteria as B. subilis and S. aureus offer bacteria-specific substitutions at positions 502, 503, 513, 538, 542, and 543. Positions 502 and 543, however, are not bacteria-specific in the Proteobacteria-gamma sequences, and therefore, drugs targeting these two positions should not be designed to act on these, organisms. The smaller analog provides a similar phylogenetic specificity pattern.

### Example 7

### The 690, 790, and 960 Stem-Loops

Components of the P-site are widely distributed in the functional skeleton. The 690 and 790 Stem-Loops, composing along with the G926 Helix the (presumed) edeine binding site, are usually considered to lie within the “platform” of the 30S subunit, while the 960 Stem-Loop lies within the “head.” The 690 and 790 Stem-Loops interact with both tRNA and initiation factors at non-overlapping sites. The 960 Stem-Loop is distinguished by the presence of an “essential” tRNA-protected base. All three of these small structures may quite easily be turned into simple stem-loop analogs. The 690 Stem-Loop contains numerous E. coli-specific bases, including G698 which is an initiation factor interaction site. The 960 Stem-Loop also offers several E. coli-specific positions, including G966, an “essential” tRNA interaction site. In contrast, the 790 Stem-Loop has little bacteria-specificity potential.

Nucleotide substitutions exist in the 690, 790, and 960 Stem-Loops for H. influenzae (proteobacteria, gamma), B. subtilis (gram positive, low GC), and S. aureus (gram positive, low GC) sequences. The 690 Stem-Loop analog will support specific targeting of S. aureus (relative to E coli) via bacteria-specific substitutions at positions 682 and 708. Compounds targeting these positions should probably not be designed to act against H. influenzae nor B. subtlis, however, since these positions loose bacteria-specificity in these organisms. The 790 Stem-Loop analog offers no clear inter-bacterial targeting potential. In contrast, the 960 Stem-Loop contains a gram positive, low GC-specific substitution at position 965, immediately adjacent to the important G966 nucleotide, thereby supporting specific targeting of these organisms.

### Example 8

### The S7 Region

The S7 region contains two P-site-associated tRNA protections (G1338 and A1339), one of which is essential (G1338). The proposed three-dimensional arrangement for this region is guided by the recently documented tertiary base pairing interaction between G944 and C1237, which effectively closes the gap between the two adjacent helical segments, leading to the proposed stacked arrangement. This structure similarly stacks the other two helical ends in the region, bulging out the single-stranded 1300 region. The entire region contains numerous S7-induced protections, suggesting that folding depends strongly on the protein. An analog seeking to circumvent this presumed protein requirement can be prepared by i) stably terminating the upper 944/1237 helix with a tetraloop, and/or ii) eliminating the 1300 single-strand, leading to more ready stacking interactions between the two remaining, tetraloop-terminated helices. This analog offers moderate bacteria-specific targeting potential via the E. coli-specific nucleotides A1239, U1335, C1336, as well as base pairs C940-G1343 and G941-C1342.

Nucleotide substitutions exist in the S7 Region analog for H. influenzae (proteobacteria, gamma), B. subtilis (gram positive, low GC), and S. aureus (gram positive, low GC) sequences. Inspection of these data suggests that gram positive, low GC bacteria may be differentially targeted by drugs targeting position 1335. In contrast, drugs targeting position 1336 should probably not be designed to act against B. subtilis nor S. aureus since this position looses bacteria-specificity in these organisms.

### Example 9

### The Spec Region

The spectinomycin region contains two nucleotides protected by the antibiotic spectinomycin (C1063 and G1064), as well as several nearby drug resistance mutation positions. This region is generally considered to be part of the 30S subunit “head,” but it has been implicated in decoding-related activities (termination) functionally, and structurally, by the discovery of a tertiary interaction linking this region with the G926 Helix. Furthermore, this region is thought to, by itself, form a binding site for spectinomycin, at least within bacteria. Interestingly, this region has also been implicated in A-site function by the presence of i) a tetracycline-resistance mutation at position 1058, and ii) two nearby nucleotides (1052 and 1054) which exhibit enhanced reactivity towards chemical probes in response to tetracycline binding. Finally, mRNA positions +8 and +9 have been crosslinked to position 1196. An analog version of the Spec Region can be prepared that adds a tetraloop termination to the distal end. This subdomain appears to offer good bacteria-specific targeting potential with G1064, a spectinomycin contact site, as well as positions 1051, 1060, 1189, 1197, 1202, 1203, and 1207 all E. coli-specific.

Nucleotide substitutions exist in the Spectinomycin Region analog for H. influenzae (proteobacteria, gamma), B. subtilis (gram positive, low GC), and S. aureus (gram positive, low GC) sequences. Gram positive, low GC bacteria may be differentially targeted by drugs targeting positions 1059 and 1198. These two positions, however, are not bacteria-specific in the proteobacteria-gamma sequences, and drugs targeting these positions should probably not be designed to target these organisms. Similarly, drugs targeting positions 1051 and 1207 should probably not be designed to act against B. subtilis nor S. aureus as these positions lose bacteria-specificity in these organisms.

Various modifications of the invention, in addition to those described herein, will be apparent to those skilled in the art from the foregoing description. Such modifications are also intended to fall within the scope of the appended claims. Each reference cited in the present application is incorporated herein by reference in its entirety.

1. A method for identifying compounds capable of binding to a molecular interaction site of 16S rRNA comprising:
(a) identifying a putative molecular interaction site of the 16S rRNA polynucleotide;
(b) providing a numerical representation of the three dimensional structure of the putative molecular interaction site;
(c) providing a compound data set comprising numerical representations of the three dimensional structure of a plurality of compounds;
(d) comparing the numerical representation of the three dimensional structure of the putative molecular interaction site with the compound data set; and
(e) creating a hierarchy of compounds ranked in accordance with the ability of the organic compounds to form physical interactions with the putative molecular interaction site thereby identifying compounds capable of binding to a molecular interaction site of 16S rRNA.

2. The method of claim 1 wherein the method is carried out in silico.

3. The method of claim 2 wherein the method is carried out using the DOCK software program.

4. The method of any of claims 1-3 wherein the putative molecular interaction site of 16S rRNA is a consensus site of the 16S rRNA polynucleotide.

5. The method of claim 4 wherein said consensus site comprises a first polynucleotide and a second polynucleotide wherein:
said first polynucleotide comprises at least fifty five nucleotides but not more than one hundred five nucleotides of SEQ ID NO:188 and comprises secondary structure defined by: a first side of a first stem comprising three nucleotides, a bulge comprising four nucleotides, a first side of a second stem comprising six nucleotides, a first side of a third stem comprising three nucleotides, a first terminal loop comprising four nucleotides, a second side of said third stem comprising three nucleotides, a bulge comprising two nucleotides, a first side of a fourth stem comprising four nucleotides, a second terminal loop comprising six nucleotides, a second side of said fourth stem comprising four nucleotides, a bulge comprising two nucleotides, a second side of said second stem comprising six nucleotides, a bulge comprising five nucleotides, and a first side of a fifth stem comprising three nucleotides; and
said second polynucleotide comprises at least eleven nucleotides but not more than sixty one nucleotides of SEQ ID NO:189 and comprises secondary structure defined by: a second side of said fifth stem comprising three nucleotides, a bulge comprising five nucleotides, and a second side of said first stem comprising three nucleotides.

6. The method of claim 5 further comprising:
(a) synthesizing the highly ranked members of said hierarchy of compounds;
(b) synthesizing polynucleotides comprising the 16S rRNA putative molecular interaction site; and
(c) contacting the putative molecular interaction site with at least one of said highly ranked members to provide a complex between the putative molecular interaction site and the member or members;
(d) ionizing said complex;
(e) fragmenting the ionized complex; and
(f) determining whether highly ranked members binds to the putative molecular interaction site of said RNA.

7. The method of claim 6 further comprising determining the strength of binding of a highly ranked member in comparison to the binding strength of other highly ranked members.

8. The method of claim 4 wherein said consensus site comprises at least fourteen nucleotides and up to sixty four nucleotides comprising SEQ ID NO:197 and comprising a secondary structure defined by: a first side of a stem comprising three nucleotides, a terminal loop comprising eight nucleotides, and a second side of said stem comprising three nucleotides.

9. The method of claim 8 further comprising:
(a) synthesizing the highly ranked members of said hierarchy of compounds;
(b) synthesizing polynucleotides comprising the 16S rRNA putative molecular interaction site; and
(c) contacting the putative molecular interaction site with at least one of said highly ranked members to provide a complex between the putative molecular interaction site and the member or members;
(d) ionizing said complex;
(e) fragmenting the ionized complex; and
(f) determining whether highly ranked members binds to the putative molecular interaction site of said RNA.

10. The method of claim 9 further comprising determining the strength of binding of a highly ranked member in comparison to the binding strength of other highly ranked members.

11. The method of claim 4 wherein said consensus site comprises a first polynucleotide and a second polynucleotide wherein:
said first polynucleotide comprises at least eight nucleotides but not more than fifty eight nucleotides of the sequence 5′-ccgcccgu-3′ and comprises secondary structure defined by: a dangling region comprising two nucleotides, a first side of a stem comprising four nucleotides wherein a first side of a first internal loop comprising one nucleotide is present between the second and third nucleotides of said first side of said stem, and a dangling region comprising one nucleotide; and
said second polynucleotide comprises at least thirty four or thirty five nucleotides but not more than eighty four or eighty five nucleotides of SEQID NO: 201 or SEQID NO: 202 and comprises secondary structure defined by: a dangling region comprising one nucleotide, a second side of said stem comprising four nucleotides wherein a second side of said first internal loop comprising two nucleotides is present between the third and fourth nucleotides of said second side of said stem, a bulge comprising four nucleotides, a first side of a second stem comprising nine nucleotides wherein a first side of a second internal loop comprising one nucleotide is optionally present between the fifth and sixth nucleotides of said first side of said second stem, a terminal loop comprising four nucleotides, and a second side of said second stem comprising nine nucleotides wherein a bulge or second side of said second internal loop comprising one nucleotide is present between the fourth and fifth nucleotides of said second side of said second stem.

12. The method of claim 11 further comprising:
(a) synthesizing the highly ranked members of said hierarchy of compounds;
(b) synthesizing polynucleotides comprising the 16S rRNA putative molecular interaction site; and
(c) contacting the putative molecular interaction site with at least one of said highly ranked members to provide a complex between the putative molecular interaction site and the member or members;
(d) ionizing said complex;
(e) fragmenting the ionized complex; and
(f) determining whether highly ranked members binds to the putative molecular interaction site of said RNA.

13. The method of claim 12 further comprising determining the strength of binding of a highly ranked member in comparison to the binding strength of other highly ranked members.

14. A method for identifying compounds capable of binding to a putative molecular interaction site of 16S rRNA comprising:
(a) identifying a putative molecular interaction site of the 16S rRNA polynucleotide;
(b) synthesizing polynucleotide comprising said putative molecular interaction site;
(c) contacting said putative molecular interaction site with a plurality of compounds predicted or suspected to be capable of interacting with said putative molecular interaction site such that a complex is formed between one or more of the compounds and the polynucleotide;
(d) ionizing said complex;
(e) fragmenting the ionized complex; and
(f) determining whether any of the compounds binds to the putative molecular interaction site of said polynucleotide.

15. The method of claim 14 further comprising determining the strength of binding of each compound in comparison to the binding strength of other compounds.


SEQUENCE LISTING (202 sequences)

SEQ ID NO: 1 - RNA, 10 nt, Artificial Sequence
  Synthetic Construct
ncgnnnnagn

SEQ ID NO: 2 - RNA, 11 nt, Artificial Sequence
  Synthetic Construct
ncgnnnnnagn

SEQ ID NO: 3 - RNA, 12 nt, Artificial Sequence
  Synthetic Construct
ncgnnnnnnagn

SEQ ID NO: 4 - RNA, 13 nt, Artificial Sequence
  Synthetic Construct
ncgnnnnnnnagn

SEQ ID NO: 5 - RNA, 14 nt, Artificial Sequence
  Synthetic Construct
ncgnnnnnnnnagn

SEQ ID NO: 6 - RNA, 15 nt, Artificial Sequence
  Synthetic Construct
ncgnnnnnnnnnagn

SEQ ID NO: 7 - RNA, 16 nt, Artificial Sequence
  Synthetic Construct
ncgnnnnnnnnnnagn

SEQ ID NO: 8 - RNA, 17 nt, Artificial Sequence
  Synthetic Construct
ncgnnnnnnnnnnnagn

SEQ ID NO: 9 - RNA, 18 nt, Artificial Sequence
  Synthetic Construct
ncgnnnnnnnnnnnnagn

SEQ ID NO: 10 - RNA, 19 nt, Artificial Sequence
  Synthetic Construct
ncgnnnnnnnnnnnnnagn

SEQ ID NO: 11 - RNA, 20 nt, Artificial Sequence
  Synthetic Construct
ncgnnnnnnnnnnnnnnagn

SEQ ID NO: 12 - RNA, 21 nt, Artificial Sequence
  Synthetic Construct
ncgnnnnnnnnnnnnnnnagn

SEQ ID NO: 13 - RNA, 22 nt, Artificial Sequence
  Synthetic Construct
ncgnnnnnnnnnnnnnnnnagn

SEQ ID NO: 14 - RNA, 23 nt, Artificial Sequence
  Synthetic Construct
ncgnnnnnnnnnnnnnnnnnagn

SEQ ID NO: 15 - RNA, 24 nt, Artificial Sequence
  Synthetic Construct
ncgnnnnnnnnnnnnnnnnnnagn

SEQ ID NO: 16 - RNA, 25 nt, Artificial Sequence
  Synthetic Construct
ncgnnnnnnnnnnnnnnnnnnnagn

SEQ ID NO: 17 - RNA, 26 nt, Artificial Sequence
  Synthetic Construct
ncgnnnnnnnnnnnnnnnnnnnnagn

SEQ ID NO: 18 - RNA, 27 nt, Artificial Sequence
  Synthetic Construct
ncgnnnnnnnnnnnnnnnnnnnnnagn

SEQ ID NO: 19 - RNA, 28 nt, Artificial Sequence
  Synthetic Construct
ncgnnnnnnnnnnnnnnnnnnnnnnagn

SEQ ID NO: 20 - RNA, 29 nt, Artificial Sequence
  Synthetic Construct
ncgnnnnnnnnnnnnnnnnnnnnnnnagn

SEQ ID NO: 21 - RNA, 30 nt, Artificial Sequence
  Synthetic Construct
ncgnnnnnnnnnnnnnnnnnnnnnnnnagn

SEQ ID NO: 22 - RNA, 31 nt, Artificial Sequence
  Synthetic Construct
ncgnnnnnnnnnnnnnnnnnnnnnnnnnagn

SEQ ID NO: 23 - RNA, 32 nt, Artificial Sequence
  Synthetic Construct
ncgnnnnnnnnnnnnnnnnnnnnnnnnnnagn

SEQ ID NO: 24 - RNA, 33 nt, Artificial Sequence
  Synthetic Construct
ncgnnnnnnnnnnnnnnnnnnnnnnnnnnnagn

SEQ ID NO: 25 - RNA, 34 nt, Artificial Sequence
  Synthetic Construct
ncgnnnnnnnnnnnnnnnnnnnnnnnnnnnnagn

SEQ ID NO: 26 - RNA, 5 nt, Artificial Sequence
  Synthetic Construct
ncgnn

SEQ ID NO: 27 - RNA, 36 nt, Artificial Sequence
  Synthetic Construct
ncgnnnnnnnnnnnnnnnnnnnnnnnnnnnnnnagn

SEQ ID NO: 28 - RNA, 37 nt, Artificial Sequence
  Synthetic Construct
ncgnnnnnnnnnnnnnnnnnnnnnnnnnnnnnnnagn

SEQ ID NO: 29 - RNA, 38 nt, Artificial Sequence
  Synthetic Construct
ncgnnnnnnnnnnnnnnnnnnnnnnnnnnnnnnnnagn

SEQ ID NO: 30 - RNA, 39 nt, Artificial Sequence
  Synthetic Construct
ncgnnnnnnnnnnnnnnnnnnnnnnnnnnnnnnnnnagn

SEQ ID NO: 31 - RNA, 40 nt, Artificial Sequence
  Synthetic Construct
ncgnnnnnnnnnnnnnnnnnnnnnnnnnnnnnnnnnnagn

SEQ ID NO: 32 - RNA, 41 nt, Artificial Sequence
  Synthetic Construct
ncgnnnnnnnnnnnnnnnnnnnnnnnnnnnnnnnnnnnagn

SEQ ID NO: 33 - RNA, 42 nt, Artificial Sequence
  Synthetic Construct
ncgnnnnnnnnnnnnnnnnnnnnnnnnnnnnnnnnnnnnagn

SEQ ID NO: 34 - RNA, 43 nt, Artificial Sequence
  Synthetic Construct
ncgnnnnnnnnnnnnnnnnnnnnnnnnnnnnnnnnnnnnnagn

SEQ ID NO: 35 - RNA, 44 nt, Artificial Sequence
  Synthetic Construct
ncgnnnnnnnnnnnnnnnnnnnnnnnnnnnnnnnnnnnnnnagn

SEQ ID NO: 36 - DNA, 45 bp, Artificial Sequence
  Synthetic Construct
ncgnnnnnnnnnnnnnnnnnnnnnnnnnnnnnnnnnnnnnnnagn

SEQ ID NO: 37 - RNA, 46 nt, Artificial Sequence
  Synthetic Construct
ncgnnnnnnnnnnnnnnnnnnnnnnnnnnnnnnnnnnnnnnnnagn

SEQ ID NO: 38 - RNA, 47 nt, Artificial Sequence
  Synthetic Construct
ncgnnnnnnnnnnnnnnnnnnnnnnnnnnnnnnnnnnnnnnnnnagn

SEQ ID NO: 39 - RNA, 48 nt, Artificial Sequence
  Synthetic Construct
ncgnnnnnnnnnnnnnnnnnnnnnnnnnnnnnnnnnnnnnnnnnnagn

SEQ ID NO: 40 - RNA, 49 nt, Artificial Sequence
  Synthetic Construct
ncgnnnnnnnnnnnnnnnnnnnnnnnnnnnnnnnnnnnnnnnnnnnagn

SEQ ID NO: 41 - RNA, 50 nt, Artificial Sequence
  Synthetic Construct
ncgnnnnnnnnnnnnnnnnnnnnnnnnnnnnnnnnnnnnnnnnnnnnagn

SEQ ID NO: 42 - RNA, 51 nt, Artificial Sequence
  Synthetic Construct
ncgnnnnnnnnnnnnnnnnnnnnnnnnnnnnnnnnnnnnnnnnnnnnnagn

SEQ ID NO: 43 - RNA, 52 nt, Artificial Sequence
  Synthetic Construct
ncgnnnnnnnnnnnnnnnnnnnnnnnnnnnnnnnnnnnnnnnnnnnnnnagn

SEQ ID NO: 44 - RNA, 53 nt, Artificial Sequence
  Synthetic Construct
ncgnnnnnnnnnnnnnnnnnnnnnnnnnnnnnnnnnnnnnnnnnnnnnnnagn

SEQ ID NO: 45 - RNA, 54 nt, Artificial Sequence
  Synthetic Construct
ncgnnnnnnnnnnnnnnnnnnnnnnnnnnnnnnnnnnnnnnnnnnnnnnnnagn

SEQ ID NO: 46 - RNA, 55 nt, Artificial Sequence
  Synthetic Construct
ncgnnnnnnnnnnnnnnnnnnnnnnnnnnnnnnnnnnnnnnnnnnnnnnnnnagn

SEQ ID NO: 47 - RNA, 56 nt, Artificial Sequence
  Synthetic Construct
ncgnnnnnnnnnnnnnnnnnnnnnnnnnnnnnnnnnnnnnnnnnnnnnnnnnnagn

SEQ ID NO: 48 - RNA, 57 nt, Artificial Sequence
  Synthetic Construct
ncgnnnnnnnnnnnnnnnnnnnnnnnnnnnnnnnnnnnnnnnnnnnnnnnnnnnagn

SEQ ID NO: 49 - RNA, 58 nt, Artificial Sequence
  Synthetic Construct
ncgnnnnnnnnnnnnnnnnnnnnnnnnnnnnnnnnnnnnnnnnnnnnnnnnnnnnagn

SEQ ID NO: 50 - RNA, 59 nt, Artificial Sequence
  Synthetic Construct
ncgnnnnnnnnnnnnnnnnnnnnnnnnnnnnnnnnnnnnnnnnnnnnnnnnnnnnnagn

SEQ ID NO: 51 - RNA, 60 nt, Artificial Sequence
  Synthetic Construct
ncgnnnnnnnnnnnnnnnnnnnnnnnnnnnnnnnnnnnnnnnnnnnnnnnnnnnnnnagn

SEQ ID NO: 52 - RNA, 61 nt, Artificial Sequence
  Synthetic Construct
ncgnnnnnnnnnnnnnnnnnnnnnnnnnnnnnnnnnnnnnnnnnnnnnnnnnnnnnnnagn

SEQ ID NO: 53 - RNA, 62 nt, Artificial Sequence
  Synthetic Construct
ncgnnnnnnnnnnnnnnnnnnnnnnnnnnnnnnnnnnnnnnnnnnnnnnnnnnnnnnnnagn

SEQ ID NO: 54 - RNA, 63 nt, Artificial Sequence
  Synthetic Construct
ncgnnnnnnnnnnnnnnnnnnnnnnnnnnnnnnnnnnnnnnnnnnnnnnnnnnnnnnnnnagn

SEQ ID NO: 55 - RNA, 64 nt, Artificial Sequence
  Synthetic Construct
ncgnnnnnnnnnnnnnnnnnnnnnnnnnnnnnnnnnnnnnnnnnnnnnnnnnnnnnnnnnnagn

SEQ ID NO: 56 - RNA, 65 nt, Artificial Sequence
  Synthetic Construct
ncgnnnnnnnnnnnnnnnnnnnnnnnnnnnnnnnnnnnnnnnnnnnnnnnnnnnnnnnnnnnagn

SEQ ID NO: 57 - RNA, 66 nt, Artificial Sequence
  Synthetic Construct
ncgnnnnnnnnnnnnnnnnnnnnnnnnnnnnnnnnnnnnnnnnnnnnnnnnnnnnnnnnnnnnagn

SEQ ID NO: 58 - DNA, 67 bp, Artificial Sequence
  Synthetic Construct
ncgnnnnnnnnnnnnnnnnnnnnnnnnnnnnnnnnnnnnnnnnnnnnnnnnnnnnnnnnnnnnnagn

SEQ ID NO: 59 - DNA, 68 bp, Artificial Sequence
  Synthetic Construct
ncgnnnnnnnnnnnnnnnnnnnnnnnnnnnnnnnnnnnnnnnnnnnnnnnnnnnnnnnnnnnnnnagn

SEQ ID NO: 60 - RNA, 69 nt, Artificial Sequence
  Synthetic Construct
ncgnnnnnnnnnnnnnnnnnnnnnnnnnnnnnnnnnnnnnnnnnnnnnnnnnnnnnnnnnnnnnnnagn

SEQ ID NO: 61 - DNA, 70 bp, Artificial Sequence
  Synthetic Construct
ncgnnnnnnnnnnnnnnnnnnnnnnnnnnnnnnnnnnnnnnnnnnnnnnnnnnnnnnnnnnnnnnnnagn

SEQ ID NO: 62 - DNA, 71 bp, Artificial Sequence
  Synthetic Construct
ncgnnnnnnnnnnnnnnnnnnnnnnnnnnnnnnnnnnnnnnnnnnnnnnnnnnnnnnnnnnnnnnnnnagn

SEQ ID NO: 63 - RNA, 72 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnagn

SEQ ID NO: 64 - RNA, 73 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnagn

SEQ ID NO: 65 - RNA, 74 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnagn

SEQ ID NO: 66 - RNA, 75 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnagn

SEQ ID NO: 67 - RNA, 76 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnagn

SEQ ID NO: 68 - RNA, 77 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnagn

SEQ ID NO: 69 - RNA, 78 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnnagn

SEQ ID NO: 70 - RNA, 79 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnnnagn

SEQ ID NO: 71 - RNA, 80 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnnnnagn

SEQ ID NO: 72 - RNA, 81 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnnnnnagn

SEQ ID NO: 73 - RNA, 82 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnnnnnnagn

SEQ ID NO: 74 - DNA, 83 bp, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnnnnnnnagn

SEQ ID NO: 75 - RNA, 84 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnnnnnnnnagn

SEQ ID NO: 76 - RNA, 85 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnnnnnnnnnagn

SEQ ID NO: 77 - RNA, 86 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnnnnnnnnnnagn

SEQ ID NO: 78 - RNA, 87 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnnnnnnnnnnnagn

SEQ ID NO: 79 - RNA, 88 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnnnnnnnnnnnnagn

SEQ ID NO: 80 - DNA, 89 bp, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnnnnnnnnnnnnnagn

SEQ ID NO: 81 - RNA, 90 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnnnnnnnnnnnnnnagn

SEQ ID NO: 82 - RNA, 91 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnnnnnnnnnnnnnnnagn

SEQ ID NO: 83 - RNA, 92 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnnnnnnnnnnnnnnnnagn

SEQ ID NO: 84 - RNA, 93 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnnnnnnnnnnnnnnnnnagn

SEQ ID NO: 85 - RNA, 94 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnnnnnnnnnnnnnnnnnnagn

SEQ ID NO: 86 - RNA, 95 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagn

SEQ ID NO: 87 - RNA, 96 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagn

SEQ ID NO: 88 - RNA, 97 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagn

SEQ ID NO: 89 - RNA, 98 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagn

SEQ ID NO: 90 - RNA, 99 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagn

SEQ ID NO: 91 - RNA, 100 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagn

SEQ ID NO: 92 - RNA, 101 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagn

SEQ ID NO: 93 - RNA, 102 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagn

SEQ ID NO: 94 - RNA, 103 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagn

SEQ ID NO: 95 - RNA, 104 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagn

SEQ ID NO: 96 - RNA, 105 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagn

SEQ ID NO: 97 - RNA, 106 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagn

SEQ ID NO: 98 - RNA, 107 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagn

SEQ ID NO: 99 - RNA, 108 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagn

SEQ ID NO: 100 - RNA, 109 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagn

SEQ ID NO: 101 - RNA, 110 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagn

SEQ ID NO: 102 - RNA, 111 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagn

SEQ ID NO: 103 - RNA, 112 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagn

SEQ ID NO: 104 - RNA, 113 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagn

SEQ ID NO: 105 - RNA, 114 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagn

SEQ ID NO: 106 - RNA, 115 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagn

SEQ ID NO: 107 - RNA, 116 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagn

SEQ ID NO: 108 - RNA, 117 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagn

SEQ ID NO: 109 - RNA, 118 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagn

SEQ ID NO: 110 - RNA, 119 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagn

SEQ ID NO: 111 - RNA, 120 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagn

SEQ ID NO: 112 - RNA, 121 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagn

SEQ ID NO: 113 - RNA, 122 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagn

SEQ ID NO: 114 - RNA, 123 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagn

SEQ ID NO: 115 - RNA, 124 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagn

SEQ ID NO: 116 - RNA, 125 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagn

SEQ ID NO: 117 - RNA, 126 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagn

SEQ ID NO: 118 - RNA, 127 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagn

SEQ ID NO: 119 - RNA, 128 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagn

SEQ ID NO: 120 - RNA, 129 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagn

SEQ ID NO: 121 - RNA, 130 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagn

SEQ ID NO: 122 - RNA, 131 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagn

SEQ ID NO: 123 - RNA, 132 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagn

SEQ ID NO: 124 - RNA, 133 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagn

SEQ ID NO: 125 - RNA, 134 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagn

SEQ ID NO: 126 - RNA, 135 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagn

SEQ ID NO: 127 - RNA, 136 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagn

SEQ ID NO: 128 - RNA, 137 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagn

SEQ ID NO: 129 - RNA, 138 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagn

SEQ ID NO: 130 - RNA, 139 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagn

SEQ ID NO: 131 - RNA, 140 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagn

SEQ ID NO: 132 - RNA, 141 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagn

SEQ ID NO: 133 - RNA, 142 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagn

SEQ ID NO: 134 - RNA, 143 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagn

SEQ ID NO: 135 - RNA, 144 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagn

SEQ ID NO: 136 - RNA, 145 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagn

SEQ ID NO: 137 - RNA, 146 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagn

SEQ ID NO: 138 - RNA, 147 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagn

SEQ ID NO: 139 - RNA, 147 nt, Artificial Sequence; identical to SEQ ID NO: 138
  Synthetic Construct

SEQ ID NO: 140 - RNA, 149 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagn

SEQ ID NO: 141 - RNA, 150 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagn

SEQ ID NO: 142 - RNA, 151 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagn

SEQ ID NO: 143 - RNA, 152 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagn

SEQ ID NO: 144 - RNA, 153 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagn

SEQ ID NO: 145 - RNA, 154 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagn

SEQ ID NO: 146 - RNA, 155 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagn

SEQ ID NO: 147 - RNA, 154 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnna

SEQ ID NO: 148 - RNA, 157 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagn

SEQ ID NO: 149 - RNA, 158 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagn

SEQ ID NO: 150 - RNA, 159 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagn

SEQ ID NO: 151 - RNA, 160 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagn

SEQ ID NO: 152 - RNA, 161 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagn

SEQ ID NO: 153 - RNA, 162 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagn

SEQ ID NO: 154 - RNA, 163 nt, Artificial Sequence
  Synthetic Construct
ncgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagn

SEQ ID NO: 155 - RNA, 10 nt, Artificial Sequence
  Synthetic Construct
nnnuaauacc

SEQ ID NO: 156 - RNA, 10 nt, Artificial Sequence
  Synthetic Construct
nnngaaannn

SEQ ID NO: 157 - RNA, 10 nt, Artificial Sequence
  Synthetic Construct
nnnnnnnnnn

SEQ ID NO: 158 - RNA, 12 nt, Artificial Sequence
  Synthetic Construct
gncnnngannnn

SEQ ID NO: 159 - RNA, 10 nt, Artificial Sequence
  Synthetic Construct
nnnaunagnu

SEQ ID NO: 160 - RNA, 33 nt, Artificial Sequence
  Synthetic Construct
gnunguuggunngguaanggcnnaccaagncnn

SEQ ID NO: 161 - RNA, 21 nt, Artificial Sequence
  Synthetic Construct
cngnncugagaggnngnncng

SEQ ID NO: 162 - RNA, 20 nt, Artificial Sequence
  Synthetic Construct
uggnacugaganacggncca

SEQ ID NO: 163 - RNA, 10 nt, Artificial Sequence
  Synthetic Construct
uccuacggga

SEQ ID NO: 164 - RNA, 24 nt, Artificial Sequence
  Synthetic Construct
nnncaauggnngnaanncugannn

SEQ ID NO: 165 - RNA, 29 nt, Artificial Sequence
  Synthetic Construct
gnnnganganggnnunngnunguaaannn

SEQ ID NO: 166 - RNA, 30 nt, Artificial Sequence
  Synthetic Construct
gnnnganganggnnunnngnunguaaannn

SEQ ID NO: 167 - RNA, 31 nt, Artificial Sequence
  Synthetic Construct
gnnnganganggnnunnnngnunguaaannn

SEQ ID NO: 168 - RNA, 42 nt, Artificial Sequence
  Synthetic Construct
nncggcnaacuncgugccagcagccgcgguaauacgnaggnn

SEQ ID NO: 169 - RNA, 10 nt, Artificial Sequence
  Synthetic Construct
nnnnacnnnn

SEQ ID NO: 170 - RNA, 11 nt, Artificial Sequence
  Synthetic Construct
gunaaannnnn

SEQ ID NO: 171 - RNA, 16 nt, Artificial Sequence
  Synthetic Construct
nnnnagnggnnnnnng

SEQ ID NO: 172 - RNA, 31 nt, Artificial Sequence
  Synthetic Construct
anaccnnungcgaaggcnnnnnncuggnnnn

SEQ ID NO: 173 - RNA, 12 nt, Artificial Sequence
  Synthetic Construct
uncgnaganaun

SEQ ID NO: 174 - RNA, 14 nt, Artificial Sequence
  Synthetic Construct
ugggnagcnaacag

SEQ ID NO: 175 - RNA, 11 nt, Artificial Sequence
  Synthetic Construct
cugguagucca

SEQ ID NO: 176 - RNA, 15 nt, Artificial Sequence
  Synthetic Construct
aggauuagauacccu

SEQ ID NO: 177 - RNA, 14 nt, Artificial Sequence
  Synthetic Construct
ggggaguacgnncg

SEQ ID NO: 178 - RNA, 12 nt, Artificial Sequence
  Synthetic Construct
agnnunaaacuc

SEQ ID NO: 179 - RNA, 38 nt, Artificial Sequence
  Synthetic Construct
auguggnuuaauucgangnnacgcgnanaaccuuaccn

SEQ ID NO: 180 - RNA, 14 nt, Artificial Sequence
  Synthetic Construct
ngggcuncacacnu

SEQ ID NO: 181 - RNA, 16 nt, Artificial Sequence
  Synthetic Construct
nnnuugacaunnnnnn

SEQ ID NO: 182 - RNA, 15 nt, Artificial Sequence
  Synthetic Construct
nnnnnnnaacaggug

SEQ ID NO: 183 - RNA, 10 nt, Artificial Sequence
  Synthetic Construct
cccuuangnn

SEQ ID NO: 184 - RNA, 14 nt, Artificial Sequence
  Synthetic Construct
ggugnugcauggnu

SEQ ID NO: 185 - RNA, 14 nt, Artificial Sequence
  Synthetic Construct
anucnucaugnccc

SEQ ID NO: 186 - RNA, 12 nt, Artificial Sequence
  Synthetic Construct
ugcauggnuguc

SEQ ID NO: 187 - RNA, 14 nt, Artificial Sequence
  Synthetic Construct
gucaanucnucaug

SEQ ID NO: 188 - RNA, 55 nt, Artificial Sequence
  Synthetic Construct
gucgucagcucgugnngugannuguuggguuaagucccgnaacgagcgcaacccn

SEQ ID NO: 189 - RNA, 11 nt, Artificial Sequence
  Synthetic Construct
gggangacguc

SEQ ID NO: 190 - RNA, 10 nt, Artificial Sequence
  Synthetic Construct
nnnacugccn

SEQ ID NO: 191 - RNA, 15 nt, Artificial Sequence
  Synthetic Construct
nggaggaaggngggg

SEQ ID NO: 192 - RNA, 12 nt, Artificial Sequence
  Synthetic Construct
nnnguuncnanc

SEQ ID NO: 193 - RNA, 11 nt, Artificial Sequence
  Synthetic Construct
gngnacucnnn

SEQ ID NO: 194 - RNA, 10 nt, Artificial Sequence
  Synthetic Construct
nnacanngng

SEQ ID NO: 195 - RNA, 10 nt, Artificial Sequence
  Synthetic Construct
cnnnnaaann

SEQ ID NO: 196 - RNA, 32 nt, Artificial Sequence
  Synthetic Construct
gngnngcnannnngnnannnnnagcnaancnn

SEQ ID NO: 197 - RNA, 14 nt, Artificial Sequence
  Synthetic Construct
nncugcaacucgnn

SEQ ID NO: 198 - RNA, 13 nt, Artificial Sequence
  Synthetic Construct
nnaucagnangnn

SEQ ID NO: 199 - RNA, 10 nt, Artificial Sequence
  Synthetic Construct
cangnnagnn

SEQ ID NO: 200 - RNA, 10 nt, Artificial Sequence
  Synthetic Construct
nnnganuggg

SEQ ID NO: 201 - RNA, 34 nt, Artificial Sequence
  Synthetic Construct
ucguaacaagguanccuannngaannugnggnug

SEQ ID NO: 202 - RNA, 35 nt, Artificial Sequence
  Synthetic Construct
ucguaacaagguanccnuannngaannugnggnug